SEQUENCE LISTING (25 sequences)

SEQ ID NO: 1 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGAC

SEQ ID NO: 4 - protein, 119 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 5 - protein, 107 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 6 - protein, 8 aa, Artificial Sequence
  Description of Artificial Sequence Synthetic peptide
GFSLTNYG

SEQ ID NO: 7 - protein, 7 aa, Artificial Sequence
  Description of Artificial Sequence Synthetic peptide
IWSGGNT

SEQ ID NO: 8 - protein, 13 aa, Artificial Sequence
  Description of Artificial Sequence Synthetic peptide
ARALTYYDYEFAY

SEQ ID NO: 9 - protein, 6 aa, Artificial Sequence
  Description of Artificial Sequence Synthetic peptide
QSIGTN

SEQ ID NO: 10 - protein, 3 aa, Artificial Sequence
  Description of Artificial Sequence Synthetic peptide
YAS

SEQ ID NO: 11 - protein, 9 aa, Artificial Sequence
  Description of Artificial Sequence Synthetic peptide
QQNNNWPTT

SEQ ID NO: 12 - protein, 25 aa, Artificial Sequence
  Description of Artificial Sequence Synthetic peptide
QVQLKQSGPGLVQPSQSLSITCTVS

SEQ ID NO: 13 - protein, 17 aa, Artificial Sequence
  Description of Artificial Sequence Synthetic peptide
VHWVRQSPGKGLEWLGV

SEQ ID NO: 14 - protein, 38 aa, Artificial Sequence
  Description of Artificial Sequence Synthetic polypeptide
DYNTPFTSRLSINKDNSKSQVFFKMNSLQSNDTAIYYC

SEQ ID NO: 15 - protein, 11 aa, Artificial Sequence
  Description of Artificial Sequence Synthetic peptide
WGQGTLVTVSA

SEQ ID NO: 16 - protein, 26 aa, Artificial Sequence
  Description of Artificial Sequence Synthetic peptide
DILLTQSPVILSVSPGERVSFSCRAS

SEQ ID NO: 17 - protein, 17 aa, Artificial Sequence
  Description of Artificial Sequence Synthetic peptide
IHWYQQRTNGSPRLLIK

SEQ ID NO: 18 - protein, 36 aa, Artificial Sequence
  Description of Artificial Sequence Synthetic polypeptide
ESISGIPSRFSGSGSGTDFTLSINSVESEDIADYYC

SEQ ID NO: 19 - protein, 10 aa, Artificial Sequence
  Description of Artificial Sequence Synthetic peptide
FGAGTKLELK

SEQ ID NO: 20 - protein, 98 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 21 - protein, 15 aa, Artificial Sequence
  Description of Artificial Sequence Synthetic peptide
EPKSCDKTHTCPPCP

SEQ ID NO: 22 - protein, 110 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 23 - protein, 105 aa, Artificial Sequence
  Description of Artificial Sequence Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 107 aa, Artificial Sequence [sequence omitted]

SEQ ID NO: 25 - protein, 107 aa, Artificial Sequence
  Description of Artificial Sequence Synthetic polypeptide
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGAC

(19) United States
(12) Patent Application Publication
(10) Pub. No.: US 2018/0154014 A1
(43) Pub. Date: Jun. 7, 2018

(54) BETA-1,6-GLUCAN CETUXIMAB ANTIBODY CONJUGATES

(71) Applicant: ImmuneXcite, Inc., Waltham, MA (US)

(72) Inventors: Gabriel Oscar Reznik, Bedford, MA (US); John James Kane, Cambridge, MA (US); James Michael Siedlecki, Burlington, MA (US); Zuzana Dostalova, Cambridge, MA (US); Isabelle Sansal-Castellano, Jamaica Plain, MA (US); Ifat Rubin-Bejerano, Newton, MA (US); Hua Miao, Newton, MA (US)

(21) Appl. No.: 15/578,343

(22) PCT Filed: Jun. 1, 2016
(86) PCT No.: PCT/US16/35346

§ 371 Date: Nov. 30, 2017

Related U.S. Application Data
(60) Provisional application No. 62/169,495, filed on Jun. 1, 2015.

(51) Int. Cl. A61K 47/68; C07K 16/28; C07K 16/30; A61P 35/00

(52) U.S. Cl. CPC A61K 47/6845; C07K 16/2863; C07K 16/30; A61P 35/00; A61K 2039/505; C07K 2317/24; C07K 2317/732; C07K 2317/94; C07K 2317/56

(57) ABSTRACT

The present invention encompasses embodiments in which cetuximab or a related cetuximab antibody is conjugated to β-1,6-glucan oligomers. Thus, the present invention includes, among other things, compositions including cetuximab conjugated to one or more β-1,6-glucan oligomers. The present invention further includes, among other things, methods of making and/or using such β-1,6-glucan conjugates. In certain embodiments, a β-1,6-glucan conjugate of the present invention is useful as a therapeutic or in a method of therapy.

### RELATED APPLICATIONS

The present application claims the benefit of U.S. Provisional Application Ser. No. 62/169,495, filed Jun. 1, 2015, the entire contents of which are incorporated herein by reference.

### BACKGROUND

Cetuximab is an epidermal growth factor receptor (EGFR) inhibitor used for the treatment of squamous cell Carcinoma of the head and neck (SCCHN) and colorectal cancer (e.g., KRAS wild-type EGFR-expressing colorectal cancer). Cetuximab has efficacy for the treatment of these types of cancer in some but not all patients and/or under some but not all conditions. There is therefore a need for new forms of cetuximab antibodies that have improved efficacy in patients and/or efficacy across a broader set of cancer patients.

### SUMMARY

The present invention encompasses embodiments in which cetuximab, or a related cetuximab antibody is conjugated to β-1,6-glucan oligomers. Thus, the present invention includes, among other things, compositions including cetuximab conjugated to one or more β-1,6-glucan oligomers. The present invention further includes, among other things, methods of making and/or using these β-1,6-glucan conjugates. In certain embodiments, a β-1,6-glucan conjugate of the present invention is useful as a therapeutic or in a method of therapy.

In certain embodiments, the present invention encompasses a composition including a cetuximab antibody conjugated to between 1 and 6 β-1,6-glucan oligomers (e.g., between 1 and 5, 1 and 4, or 1 and 3 β-1,6-glucan oligomers), wherein each β-1,6-glucan oligomer is independently comprised of between 2 and 10 glucose monomer units. In certain embodiments, each of the β-1,6-glucan oligomers is independently comprised of between 3 and 7 glucose monomer units, each of the β-1,6-glucan oligomers is independently comprised of between 3 and 5 glucose monomer units, each of the β-1,6-glucan oligomers is comprised of 5 glucose monomer units, or each of the β-1,6-glucan oligomers is comprised of 4 glucose monomer units. In certain embodiments, the cetuximab antibody is conjugated to between 2 and 4 β-1,6-glucan oligomers, e.g., to 3 β-1,6-glucan oligomers.

In certain particular embodiments, the cetuximab antibody is conjugated to between 2 and 4 β-1,6-glucan oligomers, and the β-1,6-glucan oligomers are each independently comprised of between 3 and 7 glucose monomer units. In certain particular embodiments, the cetuximab antibody is conjugated to between 2 and 4 β-1,6-glucan oligomers, and the β-1,6-glucan oligomers are each independently comprised of between 3 and 5 glucose monomer units. In certain particular embodiments, the cetuximab antibody is conjugated to between 2 and 4 β-1,6-glucan oligomers, and the β-1,6-glucan oligomers are each independently comprised of 5 glucose monomer units. In certain particular embodiments, the cetuximab antibody is conjugated to between 2 and 4 β-1,6-glucan oligomers, and the β-1,6-glucan oligomers are each independently comprised of 4 glucose monomer units. In certain particular embodiments, the cetuximab antibody is conjugated to 3 β-1,6-glucan oligomers, and the β-1,6-glucan oligomers are each independently comprised of between 3 and 7 glucose monomer units. In certain particular embodiments, the cetuximab antibody is conjugated to 3 β-1,6-glucan oligomers, and the β-1,6-glucan oligomers are each independently comprised of between 3 and 5 glucose monomer units. In certain particular embodiments, the cetuximab antibody is conjugated to 3 β-1,6-glucan oligomers, and the β-1,6-glucan oligomers are each independently comprised of 5 glucose monomer units. In certain particular embodiments, the cetuximab antibody is conjugated to 3 β-1,6-glucan oligomers, and the β-1,6-glucan oligomers are each independently comprised of 4 glucose monomer units.

In certain embodiments of the present invention, the cetuximab antibody is conjugated to the β-1,6-glucan oligomers according to Formula II:

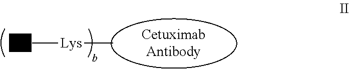

wherein: Lys is a lysine residue; b is between 1 and 6, 1 and 5, 1 and 4, or 1 and 3; and [symbol] is a compound of Formula I:

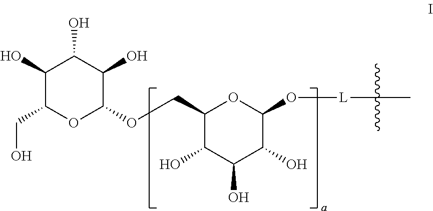

wherein: a is between 1 and 9, 1 and 8, 1 and 7, 1 and 6, 1 and 5, 1 and 4 or 1 and 3; L is a linker; and “ [symbol] ” represents a point of attachment between two atoms.

In certain embodiments of the present invention, [symbol] is a compound of Formula Ia:

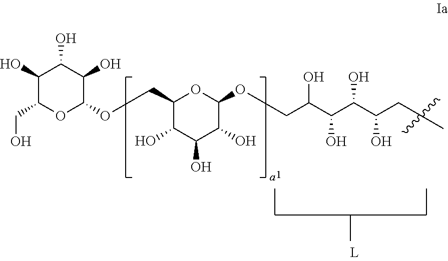

wherein: a¹ is between 1 and 9, 1 and 8, 1 and 7, 1 and 6, 1 and 5, 1 and 4 or 1 and 3; and “ [symbol] ” represents a point of attachment between two atoms.

In various embodiments of the present invention, the cetuximab antibody includes a variable domain having at least 80% identity with SEQ ID NO: 4 or SEQ ID NO: 5. In some embodiments, the cetuximab antibody includes a heavy chain variable domain having at least 80% identity with SEQ ID NO: 4 or a heavy chain having at least 80% identity with SEQ ID NO: 1. In some embodiments, the cetuximab antibody includes a light chain variable domain having at least 80% identity with SEQ ID NO: 5 or a light chain having at least 80% identity with SEQ ID NO: 2. In certain embodiments, the cetuximab antibody is cetuximab.

In some embodiments, the cetuximab antibody competes with cetuximab for binding to EGFR.

In various embodiments, the β-1,6-glucan oligomers are chemically synthesized. In various embodiments, at least 90% of the dry weight of glucan contained in the composition is β-1,6-glucan. In various embodiments, less than 10% of the dry weight of glucan contained in the composition is β-1,3-glucan. In various embodiments, the composition is substantially free of β-1,3-glucan.

The present invention further provides methods of treating a cancer associated with expression of EGFR which involve administering a composition of the present invention to a subject in need thereof. For example, in some embodiments, the methods involve administering a therapeutically effective amount of a composition that includes a cetuximab antibody conjugated to between 1 and 6 β-1,6-glucan oligomers (e.g., between 1 and 5, 1 and 4, or 1 and 3 β-1,6-glucan oligomers), where each β-1,6-glucan oligomer is independently comprised of between 2 and 10 glucose monomer units. In some embodiments, the cancer is a colorectal cancer, a KRAS wild-type EGFR-expressing colorectal cancer, a KRAS mutant EGFR-expressing colorectal cancer, a BRAF mutant EGFR-expressing colorectal cancer, a squamous cell carcinoma, a squamous cell carcinoma of the head and neck, a lung cancer, or a triple negative breast cancer.

### Definitions

Antibody: As used herein, “antibody” means an immunoglobulin molecule that recognizes and specifically binds to a target through at least one antigen recognition site within a variable, optimized, or selected region of an immunoglobulin molecule. As used herein, the term “antibody” encompasses intact polyclonal antibodies, intact monoclonal antibodies, antibody fragments (such as Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like, or any combination thereof), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as glucans, toxins, radioisotopes, and the like. As used herein, an antibody can be, e.g., an “intact antibody” or an “antibody fragment.” As used herein, “antibody” additionally encompasses various alternative formats as may be known in the art, e.g., camelid antibodies. As used herein, an antibody or intact antibody can be an immunoglobulin molecule comprising four polypeptide chains, two heavy (H) chains and two light (L) chains inter-connected by disulfide bonds. Each heavy chain comprises a heavy chain variable (V_{H}) region and a heavy chain constant region (C_{H}). The heavy chain constant region comprises three domains, C_{H}1, C_{H}2 and C_{H}3. Each light chain comprises a light chain variable (V_{L}) region and a light chain constant region (C_{L}). The V_{H} and V_{L} regions can be further subdivided into regions of hypervariability, termed complementarity determining regions (CDR), interspersed with regions that are more conserved, termed framework regions (FR). Other intact antibodies, e.g., intact camelid antibodies, are known in the art.

Antibody Fragment: As used herein, the term “antibody fragment” means a molecule comprising at least a portion derived from or having significant identity to all or a portion of an immunoglobulin protein, such as, for example, an antigen-binding or variable region of an antibody. Examples of antibody fragments include Fab, Fab′, F(ab′)₂, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc.

Glucan: As used herein, the term glucan means any polymeric molecule composed largely or entirely of glucose monomer units. A glucan can be a free molecule or may be a molecule that is conjugated with one or more other molecules, such as an antibody.

Conjugate: As used herein, the term “conjugate” refers to an antibody that is covalently linked to one or more glucans. The terms “glucan-conjugated” or “glucan-linked” as well as grammatical equivalents thereof refer to an antibody molecule that is covalently linked to one or more glucans.

Identity: As used herein, the term “identity” refers to the overall relatedness between a reference nucleic acid or amino acid sequence and one or more other nucleic acid or amino acid sequences. Identity may be expressed as a percentage. Methods for calculating percent identity are known in the art. Calculation of identity does not require that sequences be of same or similar length. Calculation of the percent identity can be performed by aligning the two sequences for optimal comparison purposes (e.g., gaps can be introduced in one or both of a first and a second nucleic acid sequences for optimal alignment and non-identical sequences can be disregarded for comparison purposes); nucleotides at corresponding nucleotide positions can then be compared. When a position in a first sequence is occupied by the same nucleotide as the corresponding position in a second sequence, then the sequences are identical at that position. The percent identity between the two sequences is a function of the number of identical positions shared by the sequences, typically taking into account, e.g., the number and/or length of any gaps introduced for optimal alignment of the sequences. The comparison of sequences and determination of percent identity between two sequences can be accomplished using a mathematical algorithm, such as BLAST®.

Treatment: As used herein, the term “treatment” (also “treat” or “treating”) refers to any administration of a therapeutic molecule (e.g., a conjugate) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Therapeutically Effective Amount: As used herein, the term “therapeutically effective amount” is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular subject. Rather, a therapeutically effective amount may be that amount that provides a particular desired pharmacological response in a significant number of subjects when administered to subjects in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular conjugate may be formulated and/or administered in a single dose. In some embodiments, a therapeutically effective amount of a particular conjugate may be formulated and/or administered in a plurality of doses, for example, as part of a dosing regimen.

### BRIEF DESCRIPTION OF THE DRAWING

FIG. 1 is a graph showing that β-1,6-glucan conjugation does not affect cetuximab binding to EGF receptor.

FIG. 2 is a graph showing that β-1,6-glucan conjugation does not affect cetuximab binding to EGF receptor.

FIG. 3 is a graph showing that β-1,6-glucan conjugation does not affect cetuximab binding to EGF receptor.

FIG. 4 is a graph showing that β-1,6-glucan conjugation does not affect cetuximab ADCC.

FIG. 5 is a graph showing that β-1,6-glucan conjugation does not affect cetuximab ADCC.

FIG. 6 is a chart showing an evaluation of anti-β-1,6-glucan IgG2 binding to a mAbXcite-cetuximab.

FIG. 7 is a chart showing an evaluation of anti-β-1,6-glucan IgG2 binding to a mAbXcite-cetuximab.

FIG. 8 is a set of two graphs showing an evaluation of anti-β-1,6-glucan IgG2 binding to a mAbXcite-cetuximab.

FIG. 9 is a set of three graphs showing an evaluation of anti-β-1,6-glucan IgG2 binding to a mAbXcite-cetuximab.

FIG. 10 is a set of seven images showing neutrophil activation by a mAbXcite-cetuximab. Neutrophil activation was observed with all β-1,6-glucan sizes.

FIG. 11 is a set of six images showing neutrophil infiltration by a mAbXcite-cetuximab.

FIG. 12 is a chart showing an evaluation of anti-β-1,6-glucan IgG2 binding to a mAbXcite-cetuximab.

FIG. 13 is a set of five graphs showing that β-1,6-glucan conjugation does not affect cetuximab binding to EGFR.

FIG. 14 is a set of five graphs showing an evaluation of anti-β-1,6-glucan IgG2 binding to a mAbXcite-cetuximab.

FIG. 15 is a set of four graphs showing an evaluation of anti-β-1,6-glucan IgG2 binding to a mAbXcite-cetuximab.

FIG. 16 is a graph showing an evaluation of anti-β-1,6-glucan IgG2 binding to a mAbXcite-cetuximab.

FIG. 17 is a chart showing an evaluation of anti-β-1,6-glucan IgG2 binding to a mAbXcite-cetuximab.

FIG. 18 is a set of two graphs showing the effect of β-1,6-glucan conjugation on antibody binding to EGF receptor and on anti-β-1,6-glucan IgG2 binding to β-1,6-glucan. Graph 1 of FIG. 18 shows the effect of conjugation on antibody binding to EGF receptor. Graph 2 of FIG. 18 shows the effect of conjugation on anti-β-1,6-glucan IgG2 binding to β-1,6-glucan.

FIG. 19 is a set of two graphs showing that a mAbXcite-cetuximab is stable in human serum. The left graph of FIG. 19 shows antibody detection in human serum. The right graph of FIG. 19 shows β-1,6-glucan detection in human serum.

FIG. 20 is a is a set of two graphs showing that a mAbXcite-cetuximab is stable in mouse serum. The left graph of FIG. 20 shows antibody detection in mouse serum. The right graph of FIG. 20 shows β-1,6-glucan detection in mouse serum.

FIG. 21 is a set of two graphs showing that a mAbXcite-cetuximab is stable in human serum. The left graph of FIG. 21 shows antibody detection in human serum. The right graph of FIG. 21 shows β-1,6-glucan detection in human serum.

FIG. 22 is a set of two graphs showing that a mAbXcite-cetuximab is stable in heat inactivated human serum. The left graph of FIG. 22 shows antibody detection in heat inactivated human serum. The right graph of FIG. 22 shows β-1,6-glucan detection in heat inactivated human serum.

FIG. 23 is a set of two graphs showing that a mAbXcite-cetuximab is stable in mouse serum. The left graph of FIG. 23 shows antibody detection in mouse serum. The right graph of FIG. 23 shows β-1,6-glucan detection in mouse serum.

FIG. 24 is a chart showing an evaluation of in vitro activity (anti-β-1,6-glucan IgG2 binding) of a mAbXcite-cetuximab tested in pharmacokinetic study.

FIG. 25 is a graph showing pharmacokinetic antibody stability of a mAbXcite-cetuximab in the absence of tumor. The graph shows that conjugation with β-1,6-glucan 5-mer does not affect cetuximab PK.

FIG. 26 is a graph showing pharmacokinetic antibody stability of a mAbXcite-cetuximab in the absence of tumor. The graph shows that conjugation with β-1,6-glucan 5-mer does not affect cetuximab PK.

FIG. 27 is a graph showing pharmacokinetic analysis of β-1,6-glucan detection in the absence of tumor.

FIG. 28 is a graph showing pharmacokinetic analysis of β-1,6-glucan detection in the absence of tumor.

FIG. 29 is a graph showing pharmacokinetic analysis of β-1,6-glucan detection in the absence of tumor.

FIG. 30 is a graph showing pharmacokinetic analysis of mAbXcite-cetuximab stability in the absence of tumor. A mAbXcite-cetuximab loaded with β-1,6-glucan 5-mer was stable based on PK with load up to 2.4.

FIG. 31 is a graph showing pharmacokinetic analysis of mAbXcite-cetuximab stability in the absence of tumor.

FIG. 32 is a graph showing pharmacokinetic analysis of mAbXcite-cetuximab stability in the absence of tumor.

FIG. 33 is a graph showing pharmacokinetic analysis of β-1,6-glucan detection in the absence of tumor.

FIG. 34 is a graph showing pharmacokinetic analysis of β-1,6-glucan detection in the absence of tumor.

FIG. 35 is a graph showing pharmacokinetic analysis of β-1,6-glucan detection in the absence of tumor.

FIG. 36 is a set of two graphs showing pharmacokinetics of a mAbXcite-cetuximab in tumor bearing animals. The left graph shows mAbXcite-cetuximab antibody detection in tumor bearing animals. The right graph shows β-1,6-glucan detection in tumor bearing animals.

FIG. 37 is a set of two graphs showing pharmacokinetics of a mAbXcite-cetuximab in tumor bearing animals. The left graph shows mAbXcite-cetuximab antibody detection in tumor bearing animals. The right graph shows β-1,6-glucan detection in tumor bearing animals.

FIG. 38 is a set of two graphs showing pharmacokinetic analysis of β-1,6-glucan detection in absence of tumor or in tumor bearing mice. The left graph shows pharmacokinetic analysis of β-1,6-glucan detection in tumor bearing mice. The left graph shows pharmacokinetic analysis of β-1,6-glucan detection in absence of tumor.

FIG. 39 is a set of two graphs showing pharmacokinetic analysis of a mAbXcite-cetuximab. The left graph shows mAbXcite-cetuximab antibody detection. The right graph shows β-1,6-glucan detection.

FIG. 40 is a set of two graphs showing a pharmacokinetic analysis of mAbXcite-cetuximab accumulation with twice weekly treatment. The left graph shows mAbXcite-cetuximab antibody detection. The right graph shows β-1,6-glucan detection. No accumulation was observed with twice weekly treatment.

FIG. 41 is a set of two graphs showing a pharmacokinetic analysis of a mAbXcite-cetuximab in tumor bearing animals. The left graph shows antibody stability. The right graph shows β-1,6-glucan detection.

FIG. 42 is a set of two graphs showing a pharmacokinetic analysis of mAbXcite-cetuximab stability in absence of tumor or in tumor bearing mice. The left graph shows antibody stability in mice with tumor. The right graph shows antibody stability in absence of tumor.

FIG. 43 is a set of two graphs showing a pharmacokinetic analysis of mAbXcite-cetuximab in absence of tumor or in tumor bearing mice. The left graph shows β-1,6-glucan detection in tumor bearing mice. The right graph shows β-1,6-glucan detection in absence of tumor.

FIG. 44 is a set of two graphs showing a pharmacokinetic analysis comparing mAbXcite-cetuximab having DBCO or direct conjugation chemistry. The left graph shows antibody stability. The right graph shows β-1,6-glucan detection.

FIG. 45 is a graph showing mAbXcite-cetuximab efficacy in a BRAF mutant colorectal tumor model.

FIG. 46 is a graph showing mAbXcite-cetuximab efficacy in a BRAF mutant colorectal tumor model.

FIG. 47 is a graph showing mAbXcite-cetuximab efficacy in a BRAF mutant colorectal tumor model.

FIG. 48 is a graph showing mAbXcite-cetuximab efficacy in a KRAS mutant colorectal tumor model.

FIG. 49 is a graph showing mAbXcite-cetuximab efficacy in a KRAS mutant colorectal tumor model.

FIG. 50 is a graph showing that a mAbXcite-cetuximab inhibits KRAS mutant colorectal tumor growth.

FIG. 51 is a graph showing that a mAbXcite-cetuximab inhibits KRAS mutant colorectal tumor growth.

FIG. 52 is a graph showing that a mAbXcite-cetuximab inhibits KRAS mutant colorectal tumor growth.

FIG. 53 is a graph showing that a mAbXcite-cetuximab inhibits KRAS mutant colorectal tumor growth.

FIG. 54 is a graph showing that a mAbXcite-cetuximab inhibits KRAS mutant colorectal tumor growth.

FIG. 55 is a graph showing that a mAbXcite-cetuximab inhibits KRAS mutant colorectal tumor growth.

FIG. 56 is a graph showing that a mAbXcite-cetuximab inhibits KRAS mutant colorectal tumor growth (mean).

FIG. 57 is a graph showing that a mAbXcite-cetuximab inhibits KRAS mutant colorectal tumor growth (median).

FIG. 58 is a graph showing that a mAbXcite-cetuximab increases survival in a KRAS mutant colorectal cancer xenograft model.

FIG. 59 is a set of six graphs showing tumor regression or stasis is observed with direct conjugate mAbXcite-cetuximab and correlates with immune memory. Individual graphs represent individual mice.

FIG. 60 is a set of five graphs showing no tumor regression or stasis with 4-mer DBCO mAbXcite-cetuximab conjugates. Individual graphs represent individual mice.

FIG. 61 is a graph showing evidence for involvement of T cells in stasis.

FIG. 62 is a graph showing KRAS mutant colorectal tumor growth.

FIG. 63 is a graph showing KRAS mutant colorectal tumor growth.

FIG. 64 is a graph showing KRAS mutant colorectal tumor growth.

FIG. 65 is a graph showing KRAS mutant colorectal tumor growth.

FIG. 66 is a graph showing mean tumor growth.

FIG. 67 is a graph showing median tumor growth.

### DETAILED DESCRIPTION

The present invention encompasses embodiments in which cetuximab or a related cetuximab antibody is conjugated to β-1,6-glucan oligomers. Thus, the present invention includes, among other things, compositions including cetuximab conjugated to one or more β-1,6-glucan oligomers. The present invention further includes, among other things, methods of making and/or using these β-1,6-glucan conjugates. In certain embodiments, a β-1,6-glucan conjugate of the present invention is useful as a therapeutic or in a method of therapy.

### Cetuximab and Cetuximab Antibodies

Cetuximab is an IgG1 mouse-human chimeric monocolonal antibody that targets epidermal growth factor receptor (EGFR). Cetuximab is used for the treatment of metastatic colorectal cancer, metastatic non-small cell lung cancer and head and neck cancer.

Cetuximab includes two heavy chains and two light chains. Cetuximab heavy chain and light chain sequences are known in the art. For instance, a cetuximab heavy chain or light chain can have a cetuximab heavy chain sequence or cetuximab light chain sequence as disclosed in any of (1) Li et al., Cancer Cell 7:301-11, 2005; (2) Dubois et al., Anal. Chem.; 80:1737-45, 2008; (3) the art-recognized IMGT database, sequence available online at www.imgt.org/3Dstructure-DB/cgi/details.cgi?pdbcode=7906; and/or (4) Ayoub et al., mAbs 5(5): 699-710, 2013 (inclusive of supplemental material); each of which is hereby incorporated by reference in its entirety. Table 1 provides certain cetuximab sequences that were obtained from these sources. Of note, SEQ ID NO: 2 is a cetuximab kappa light chain sequence from the IMGT database (supra) while SEQ ID NO: 3 is an alternative cetuximab kappa light chain sequence from Ayoub et al. (supra). SEQ ID NOs: 24 and SEQ ID NO: 25 are, respectively, the constant domains of SEQ ID NO: 2 and SEQ ID NO: 3.

TABLE 1
Cetuximab Sequences

| SEQ ID NO. | Sequence |
|---|---|
| Cetuximab IgG1 | QVQLKQSGPGLVQPSQSLSITCTVSGFSLTNYGVHWVRQSPGK |
| Heavy Chain | GLEWLGVIWSGGNTDYNTPFTSRLSINKDNSKSQVFFKMNSLQ |
| (SEQ ID NO: 1) | SNDTAIYYCARALTYYDYEFAYWGQGTLVTVSAASTKGPSVFP |
| | LAPSSKSTSGGTAALGCLVKDYFPEPVTVSWNSGALTSGVHTFP |
| | AVLQSSGLYSLSSVVTVPSSSLGTQTYICNVNHKPSNTKVDKRV |
| | EPKSCDKTHTCPPCPAPELLGGPSVFLFPPKPKDTLMISRTPEVT |
| | CVVVDVSHEDPEVKFNWYVDGVEVHNAKTKPREEQYNSTYRV |
| | VSVLTVLHQDWLNGKEYKCKVSNKALPAPIEKTISKAKGQPRE |
| | PQVYTLPPSREEMTKNQVSLTCLVKGFYPSDIAVEWESNGQPEN |
| | NYKTTPPVLDSDGSFFLYSKLTVDKSRWQQGNVFSCSVMHEAL |
| | HNHYTQKSLSLSPGK |
| Cetuximab Kappa | DILLTQSPVILSVSPGERVSFSCRASQSIGTNIHWYQQRTNGSPRL |
| Light Chain (IMGT) | LIKYASESISGIPSRFSGSGSGTDFTLSINSVESEDIADYYCQQNN |
| (SEQ ID NO: 2) | NWPTTFGAGTKLELKRTVAAPSVFIFPPSDEQLKSGTASVVCLL |
| | NNFYPREAKVQWKVDNALQSGNSQESVTEQDSKDSTYSLSSTL |
| | TLSKADYEKHKVYACEVTHQGLSSPVTKSFNRGEC |
| Cetuximab Kappa | DILLTQSPVILSVSPGERVSFSCRASQSIGTNIHWYQQRTNGSPRL |
| Light Chain (Ayoub | LIKYASESISGIPSRFSGSGSGTDFTLSINSVESEDIADYYCQQNN |
| et al.) | NWPTTFGAGTKLELKRTVAAPSVFIFPPSDEQLKSGTASVVCLL |
| (SEQ ID NO: 3) | NNFYPREAKVQWKVDNALQSGNSQESVTEQDSKDSTYSLSSTL |
| | TLSKADYEKHKVYACEVTHQGLSSPVTKSFNRGAC |
| Cetuximab Heavy | QVQLKQSGPGLVQPSQSLSITCTVSGFSLTNYGVHWVRQSPGK |
| chain variable domain | GLEWLGVIWSGGNTDYNTPFTSRLSINKDNSKSQVFFKMNSLQ |
| (1-119) | SNDTAIYYCARALTYYDYEFAYWGQGTLVTVSA |
| (SEQ ID NO: 4) | |
| Cetuximab Kappa | DILLTQSPVILSVSPGERVSFSCRASQSIGTNIHWYQQRTNGSPRL |
| Light Chain Variable | LIKYASESISGIPSRFSGSGSGTDFTLSINSVESEDIADYYCQQNN |
| Domain (1-108) | NWPTTFGAGTKLELK |
| (SEQ ID NO: 5) | |
| Cetuximab Heavy | GFSLTNYG |
| Chain CDR1 | |
| (SEQ ID NO: 6) | |
| Cetuximab Heavy | IWSGGNT |
| Chain CDR2 | |
| (SEQ ID NO: 7) | |
| Cetuximab Heavy | ARALTYYDYEFAY |
| Chain CDR3 | |
| (SEQ ID NO: 8) | |
| Cetuximab Kappa | QSIGTN |
| Light Chain CDR1 | |
| (SEQ ID NO: 9) | |
| Cetuximab Kappa | YAS |
| Light Chain CDR2 | |
| (SEQ ID NO: 10) | |
| Cetuximab Kappa | QQNNNWPTT |
| Light Chain CDR3 | |
| (SEQ ID NO: 11) | |
| Cetuximab Heavy | QVQLKQSGPGLVQPSQSLSITCTVS |
| Chain FW1 | |
| (SEQ ID NO: 12) | |
| Cetuximab Heavy | VHWVRQSPGKGLEWLGV |
| Chain FW2 | |
| (SEQ ID NO: 13) | |
| Cetuximab Heavy | DYNTPFTSRLSINKDNSKSQVFFKMNSLQSNDTAIYYC |
| Chain FW3 | |
| (SEQ ID NO: 14) | |
| Cetuximab Heavy | WGQGTLVTVSA |
| Chain FW4 | |
| (SEQ ID NO: 15) | |
| Cetuximab Kappa | DILLTQSPVILSVSPGERVSFSCRAS |
| Light Chain FW1 | |
| (SEQ ID NO: 16) | |
| Cetuximab Kappa | IHWYQQRTNGSPRLLIK |
| Light Chain FW2 | |
| (SEQ ID NO: 17) | |
| Cetuximab Kappa | ESISGIPSRFSGSGSGTDFTLSINSVESEDIADYYC |
| Light Chain FW3 | |
| (SEQ ID NO: 18) | |
| Cetuximab Kappa | FGAGTKLELK |
| Light Chain FW4 | |
| (SEQ ID NO: 19) | |
| Cetuximab Heavy | ASTKGPSVFPLAPSSKSTSGGTAALGCLVKDYFPEPVTVSWNSG |
| chain CH1 constant | ALTSGVHTFPAVLQSSGLYSLSSVVTVPSSSLGTQTYICNVNHKP |
| domain (120-217) | SNTKVDKRV |
| (SEQ ID NO: 20) | |
| Cetuximab Heavy | EPKSCDKTHTCPPCP |
| chain hinge domain | |
| (218-232) | |
| (SEQ ID NO: 21) | |
| Cetuximab Heavy | APELLGGPSVFLFPPKPKDTLMISRTPEVTCVVVDVSHEDPEVKF |
| chain CH2 constant | NWYVDGVEVHNAKTKPREEQYNSTYRVVSVLTVLHQDWLNG |
| domain (233-342) | KEYKCKVSNKALPAPIEKTISKAK |
| (SEQ ID NO: 22) | |
| Cetuximab Heavy | GQPREPQVYTLPPSREEMTKNQVSLTCLVKGFYPSDIAVEWESN |
| chain CH3 constant | GQPENNYKTTPPVLDSDGSFFLYSKLTVDKSRWQQGNVFSCSV |
| domain (343-447) | MHEALHNHYTQKSLSLSP |
| (SEQ ID NO: 23) | |
| Cetuximab Kappa | RTVAAPSVFIFPPSDEQLKSGTASVVCLLNNFYPREAKVQWKV |
| Light Chain Constant | DNALQSGNSQESVTEQDSKDSTYSLSSTLTLSKADYEKHKVYA |
| Domain (108-214) | CEVTHQGLSSPVTKSFNRGEC |
| (IMGT) | |
| (SEQ ID NO: 24) | |
| Cetuximab Kappa | RTVAAPSVFIFPPSDEQLKSGTASVVCLLNNFYPREAKVQWKV |
| Light Chain Constant | DNALQSGNSQESVTEQDSKDSTYSLSSTLTLSKADYEKHKVYA |
| Domain (108-214) | CEVTHQGLSSPVTKSFNRGAC |
| (Ayoub et al.) | |
| (SEQ ID NO: 25) | |

As used herein, the term “cetuximab antibody” encompasses cetuximab and any antibody or antibody fragment that recognizes and specifically binds EGFR and has at least a heavy chain variable domain or light chain variable domain having at least 80% identity (e.g., at least 85%, 90%, 91%, 92%, 93%, 94%, 95%, 96%, 97%, 98%, 99%, or 100% identity) to a corresponding sequence of cetuximab (i.e., SEQ ID NO:4 or SEQ ID NO: 5). In some instances, a cetuximab antibody includes two such variable domains, three such variable domains, four such variable domains, two such heavy chain variable domains, two such light chain variable domains, and/or two such heavy chain variable domains and two such light chain variable domains. In some instances, a cetuximab antibody includes a heavy chain or light chain having at least 80% identity (e.g., at least 85%, 90%, 91%, 92%, 93%, 94%, 95%, 96%, 97%, 98%, 99%, or 100% identity) to a corresponding sequence of cetuximab (i.e., one or more of SEQ ID NOs: 1-3). In some instances, a cetuximab antibody includes two such chains, three such chains, four such chains, two such heavy chains, two such light chains, and/or two such heavy chains and two such light chains. Thus, a cetuximab antibody may be, e.g., an intact antibody, antibody fragment (such as a Fab, Fab′, F(ab′)₂, Fd, or Fv), single chain Fv (scFv), or multispecific antibody such as a bispecific antibody.

In various instances of the present invention, a cetuximab antibody includes a heavy chain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 1.

In various instances of the present invention, a cetuximab antibody includes a light chain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 2 or SEQ ID NO: 3.

In various instances of the present invention, a cetuximab antibody includes a heavy chain that includes at least one heavy chain variable domain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 4.

In various instances of the present invention, a cetuximab antibody includes a light chain that includes at least one light chain variable domain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 5

In various instances of the present invention, a cetuximab antibody includes a CDR sequence having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 6, SEQ ID NO: 7, or SEQ ID NO: 8. In certain instances a cetuximab antibody includes a heavy chain including such a CDR sequence. In certain instances a cetuximab antibody includes two such heavy chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 6 and SEQ ID NO: 7. In certain instances a cetuximab antibody includes a heavy chain including such CDR sequences. In certain instances a cetuximab antibody includes two such heavy chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 6 and SEQ ID NO: 8. In certain instances a cetuximab antibody includes a heavy chain including such CDR sequences. In certain instances a cetuximab antibody includes two such heavy chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 7 and SEQ ID NO: 8. In certain instances a cetuximab antibody includes a heavy chain including such CDR sequences. In certain instances a cetuximab antibody includes two such heavy chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 6, SEQ ID NO: 7, and SEQ ID NO: 8. In certain instances a cetuximab antibody includes a heavy chain including such CDR sequences. In certain instances a cetuximab antibody includes two such heavy chains.

In various instances of the present invention, a cetuximab antibody includes a CDR sequence having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 9, SEQ ID NO: 10, or SEQ ID NO: 11. In certain instances a cetuximab antibody includes a light chain including such a CDR sequence. In certain instances a cetuximab antibody includes two such light chains.

In various instances of the present invention, a cetuximab antibody includes a CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 9 and SEQ ID NO: 10. In certain instances a cetuximab antibody includes a light chain including such CDR sequences. In certain instances a cetuximab antibody includes two such light chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 9 and SEQ ID NO: 11. In certain instances a cetuximab antibody includes a light chain including such CDR sequences. In certain instances a cetuximab antibody includes two such light chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% identity to each of SEQ ID NO: 10 and SEQ ID NO: 11. In certain instances a cetuximab antibody includes a light chain including such CDR sequences. In certain instances a cetuximab antibody includes two such light chains.

In various instances of the present invention, a cetuximab antibody includes CDR sequences having at least 80%, at least 85%, at least 90%, at least 91%, at least 92%, at least 93%, at least 94%, at least 95%, at least 96%, at least 97%, at least 98%, or at least 99% homology or identity to each of SEQ ID NO: 9, SEQ ID NO: 10, and SEQ ID NO: 11. In certain instances a cetuximab antibody includes a light chain including such CDR sequences. In certain instances a cetuximab antibody includes two such light chains.

In various instances of the present invention, a cetuximab antibody includes a heavy chain that includes at least one FW domain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 12, SEQ ID NO: 13, SEQ ID NO: 14, or SEQ ID NO: 15.

In various instances of the present invention, a cetuximab antibody includes a light chain that includes at least one FW domain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 16, SEQ ID NO: 17, SEQ ID NO: 18, or SEQ ID NO: 19.

In various instances of the present invention, a cetuximab antibody includes a heavy chain that includes at least one constant or hinge domain having at least 80%, at least 85%, at least 90%, at least 91%, at least 92%, at least 93%, at least 94%, at least 95%, at least 96%, at least 97%, at least 98%, or at least 99% identity to the sequence of one or more of SEQ ID NO: 20, SEQ ID NO: 21, SEQ ID NO: 22, or SEQ ID NO: 23.

In various instances of the present invention, a cetuximab antibody includes a light chain that includes at least one constant domain having at least 80%, at least 85%, at least 90%, at least 91%, at least 92%, at least 93%, at least 94%, at least 95%, at least 96%, at least 97%, at least 98%, or at least 99% identity to the sequence of SEQ ID NO: 24 or SEQ ID NO: 25.

Various antibodies or antibody fragments as described herein incorporate one or more amino acid mutations, e.g., one or more amino acid substitutions, in a sequence corresponding to any of SEQ ID NOs: 1-25. In some embodiments the one or more amino acid substitutions may be conservative substitutions as is known in the art. Various heavy chains and light chains described herein can be utilized in the production of an antibody, e.g., a monocolonal antibody including two heavy chains and light chains.

### β-1,6-Glucan Oligomers

A β-1,6-glucan oligomer of the present invention can be derived from or synthesized from any source and/or by any procedure, e.g., any source and/or by any procedure known in the art.

In some embodiments, the β-1,6-glucan oligomer is derived from a lichen, which in one embodiment is from the genus Umbilicariaceae (e.g., from U. pustulata and U. hirsute, U. angulata, U. caroliniana, or U. polyphylla). In some embodiments, the β-1,6-glucan is derived from a fungus, which in one embodiment is from the genus Candida (e.g., from C. albicans). Other organisms from which the glucan may be derived include Coccidioides immitis, Trichophyton verrucosum, Blastomyces dermatidis, Cryptococcus neoformans, Histoplasma capsulatum, Saccharomyces cerevisiae, Paracoccidioides brasiliensis, Botryosphaeria rhodina, Lasiodiplodia theobromae, and Pythiumn insidiosum. Pure β-glucans are commercially available, e.g., pustulan is a β-1,6-glucan purified from Umbilicaria papullosa which is available from Calbiochem and Elicityl. β-glucans can also be purified from fungal cell walls in various ways, for example, as described in Tokunaka et al., Carbohydr. Res. 316:161-172, 1999, and the product may be enriched for β-1,6-glucan moieties by methods as are known in the art. In some embodiments, a β-1,6-glucan may be isolated from an organism and then chemically or enzymatically altered, for example, to increase solubility. Indeed, full-length native glucans are insoluble and have a molecular weight in the megadalton range. In some embodiments, this invention uses soluble β-1,6-glucan oligomers. In some embodiments, solubilization may be achieved by fragmenting long insoluble glucans. This may be achieved by, for example, hydrolysis or, in some embodiments, by digestion with a glucanase (e.g., with a β-1,3 glucanase or limited digestion with a (3-1,6 glucanase).

In some embodiments, the β-1,6-glucan oligomer is chemically synthesized, as is known in the art. In an example embodiment, the β-1,6-glucan oligomer is synthesized from glucose monomers joined via glycolysation reactions. The length of the β-1,6-glucan oligomer is controlled by selecting the number of “building blocks” to use. In an example embodiment, three different “building blocks” can be selected. from:

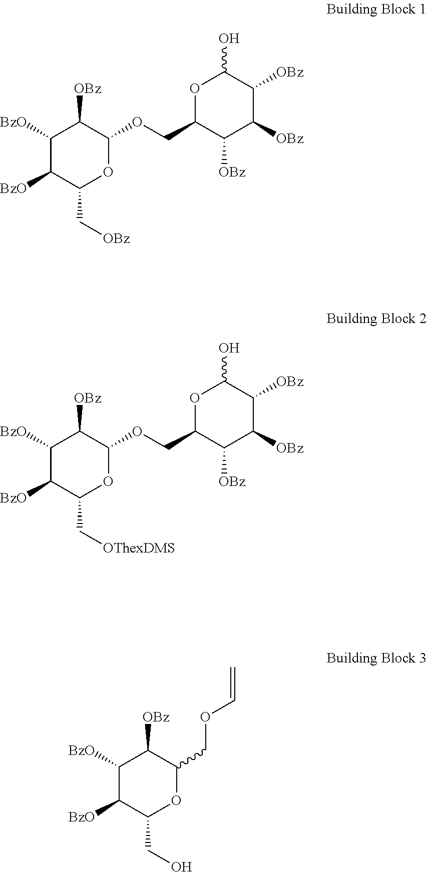

In an example embodiment, the glucose monomers or building blocks are joined via Schmidt reaction conditions. Exemplary Schmidt reaction conditions include, but are not limited to, converting a free hydroxyl group of the glucan monomer to its respective trichloroacetimidate and subsequent reaction with the free hydroxyl group of another glucose monomer or oligosaccharide in the presence of BF₃.OEt₂ or TMSOTf.

In another example embodiment, the free hydroxyl groups of the glucose monomer are selectively protected. In another example embodiment, the free hydroxyl groups are protected by reaction with benzoyl chloride or thexyldimethylsilyl chloride.

In another aspect, the present invention encompasses certain intermediate compounds which are represented by structural Formula V and that can be used in the chemical synthesis of certain β-1,6-glucan oligomers or oligomer precursors:

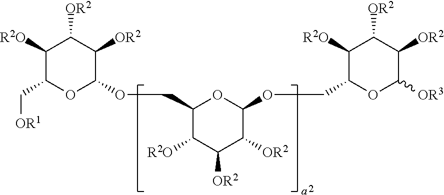

wherein

R¹ is hydrogen or a hydroxyl protecting group;

R² is hydrogen or a hydroxyl protecting group;

R³ is hydrogen, a hydroxyl protecting group,

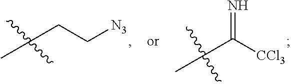

and

a² is between 0 and 8.

Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably hydroxyl protecting groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenyl sulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, dimethylthexyl silyl, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers. It is to be understood that any chemical terms used herein are intended to have their ordinary meaning as commonly used in the chemical arts.

Accordingly, in an example embodiment, R¹ is hydrogen. In another embodiment, R¹ is a hydroxyl protecting group. In another embodiment, R¹ is a hydroxyl protecting group, and the hydroxyl protecting group is dimethylthexyl silyl (“ThexDMS”). In another embodiment, R¹ is a hydroxyl protecting group, and the hydroxyl protecting group is benzoyl formate (“Bz”). In another embodiment, R² is hydrogen. In another embodiment, R² is a hydroxyl protecting group. In another embodiment, R² is a hydroxyl protecting group, and the hydroxyl protecting group is benzoyl formate.

In another embodiment, R³ is hydrogen. In another embodiment, R³ is

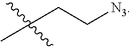 [SMILES: *CCN=[N+]=[N-]]

In another embodiment, R³ is

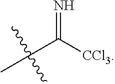

In another embodiment, R² is a hydroxyl protecting group. In another embodiment, R³ is a hydroxyl protecting group, and the hydroxyl protecting group is benzoyl formate. In another embodiment, In another embodiment, R³ is a hydroxyl protecting group, and the hydroxyl protecting group is allyl ether.

In another embodiment a² is 0, 1, 2, 3, 4, 5, 6, 7, or 8.

As used herein, [symbol] appearing on a structure and joining a functional group to the structure in the position of a bond generally indicates a mixture of, or either of, the possible isomers, e.g., containing (R)- and (S)-stereochemistry. For example,

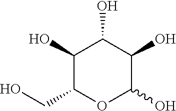

means containing either, or both of:

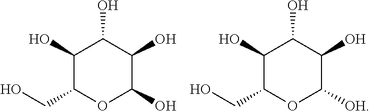

As used herein, [symbol] appearing across a bond indicates a point of attachment between two atoms. For example,

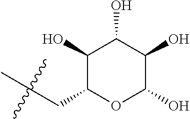 [SMILES: *C[C@H]1O[C@@H](O)[C@H](O)[C@@H](O)[C@@H]1O]

means that the glucose ring above is bound to an undepicted structure on which it is a substituent.

In some embodiments of the present invention, a β-1-6 glucan oligomer of the present invention includes a low molecular weight β-1-6 glucan oligomer, e.g., a β-1-6 glucan oligomer containing 10 or fewer (e.g., 9, 8, 7, 6, 5, 4, 3 or 2) glucose monomer units. In some embodiments, a composition of the present invention comprises β-1,6-glucan oligomers which comprise, consist essentially of or consist of low molecular weight β-1,6-glucan oligomers, e.g., β-1-6 glucan oligomers containing 10 or fewer (e.g., 9, 8, 7, 6, 5, 4, 3 or 2) glucose monomer units. In certain embodiments, at least 80%, 90%, 95%, 98%, 99% or 100% of the β-1,6-glucan oligomers contained in a composition of the invention by weight is low molecular weight β-1,6-glucan oligomers, e.g., β-1-6 glucan oligomers containing 10 or fewer (e.g., 9, 8, 7, 6, 5, 4, 3 or 2) glucose monomer units. In certain embodiments, “weight” refers to “dry weight”.

In certain embodiments, at least 80%, 90%, 95%, 98%, 99% or 100% of the glucan contained in a composition of the invention by weight is β-1,6 glucan. In certain embodiments, “weight” refers to “dry weight”. In certain embodiments, less than 20%, 10%, 5%, 2% or 1% of the glucan contained in a composition of the invention by weight is β-1,3 glucan. In certain embodiments, “weight” refers to “dry weight”.

### Conjugates

Cetuximab, or any cetuximab antibody disclosed herein, may be conjugated to one or more β-1,6-glucan oligomers. The present application relates, among other things, to the length of β-1,6-glucan oligomers to be conjugated to cetuximab or a cetuximab antibody, the load of β-1,6-glucan oligomers to be conjugated to cetuximab or a cetuximab antibody (i.e., the number of β-1,6-glucan oligomers to be conjugated to each antibody), and to the type of conjugation by which β-1,6-glucan oligomers are linked with cetuximab or a cetuximab antibody.

### Glucan Length

In some embodiments, a conjugate of the present invention includes a β-1,6-glucan oligomer which is comprised of between 2 and 10 glucose monomer units (e.g., 2, 3, 4, 5, 6, 7, 8, 9, or 10 glucose monomer units). In particular embodiments, a conjugate of the present invention includes a β-1,6-glucan oligomer which is comprised of between 3 to 10, 3 to 9, 3 to 8, 3 to 7, 3 to 6, 3 to 5, 3 to 4, 4 to 10, 4 to 9, 4 to 8, 4 to 7, 4 to 6 or 4 to 5 glucose monomer units.

In some embodiments, a conjugate of the present invention includes a β-1,6-glucan oligomer covalently linked to cetuximab or a cetuximab antibody via a linker L as shown in Formula I:

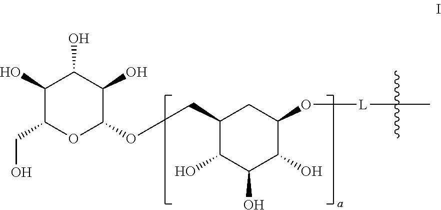

wherein a is between 1 and 9, 1 and 8, 1 and 7, 1 and 6, 1 and 5, 1 and 4 or 1 and 3, L is a linker, and “ [symbol] ” represents a point of attachment between two atoms (i.e., an atom of the linker and an atom of cetuximab or a cetuximab antibody). In some embodiments, the linker L can be a ring-opened glucose monomer as shown in Formula Ia:

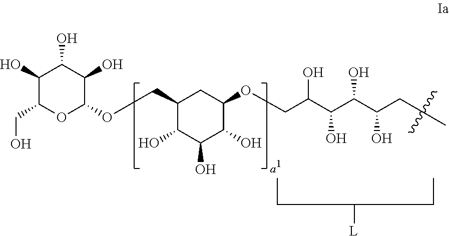

wherein a¹ is between 1 and 9, 1 and 8, 1 and 7, 1 and 6, 1 and 5, 1 and 4 or 1 and 3, and “ [symbol] ” represents a point of attachment between two atoms.

When a conjugate includes several β-1,6-glucan oligomers or when discussing a composition that includes a population of conjugates, it is to be understood that the aforementioned values and ranges may refer to the actual or average number of glucose monomer units that are present in the conjugated β-1,6-glucan oligomers. The length of the β-1,6-glucan oligomers (defined based on the number of glucose monomer units) may be a whole number, e.g., when referring to a single β-1,6-glucan oligomer or a population of β-1,6-glucan oligomers each having the same length. The length of the β-1,6-glucan oligomers may also be a whole number when referring to the length of a population of β-1,6-glucan oligomers wherein the whole number length is representative of the actual length of at least 90% of the β-1,6-glucan oligomers in the population (e.g., at least 91%, 92%, 93%, 94%, 95%, 96%, 97%, 98%, 99%, or 100% of the β-1,6-glucan oligomers in the population). The length of the β-1,6-glucan oligomers may be presented as a fraction when a population includes a mixture of β-1,6-glucan oligomers having two or more different lengths. The fraction may be indicative of a hypothetical, expected, approximate, or measured average length of β-1,6-glucan oligomers in the population. Accordingly, a length expressed as being between two whole numbers encompasses any intervening fraction of a whole number.

Various methods of determining the number of monomeric glucose units in a β-1,6-glucan oligomer are known in the art. In various instances, the number of monomeric glucose units in a β-1,6-glucan oligomer is provided or determined prior to conjugation with cetuximab or a cetuximab antibody. For instance, in some instances, a β-1,6-glucan oligomer or β-1,6-glucan oligomer precursor is synthesized to have a particular known length. Example 21 describes synthesis of gentiopentose. Example 22 describes synthesis of 2-azidoethylgentiotetrose. Example 23 describes synthesis of 2-azidoethylgentiohexose. It will be appreciated that β-1,6-glucan oligomers and other β-1,6-glucan oligomer precursors having different lengths can be made in accordance with known synthetic methods (e.g., those described herein) using these and other building blocks that are described herein. In certain instances, β-1,6-glucan oligomers having a particular number (or range) of monomeric glucose units are isolated from a population of oligomers, e.g., a population of oligomers derived from pustulan, e.g., by breakdown or modification of pustulan. In various instances a population of β-1,6-glucan oligomers each having a particular number of monomeric glucose units is provided and the number of monomeric glucose units per oligomer is determined by chromatography (e.g., size exclusion chromatograph) and/or mass spectrometry (e.g., MALDI). In various instances, a population of β-1,6-glucan oligomers including oligomers having various numbers of monomeric glucose units is provided and the number of monomeric glucose units per oligomer is determined by chromatography (e.g., size exclusion chromatograph) and/or mass spectrometry (e.g., MALDI). In various instances, one or more β-1,6-glucan oligomers having a particular number of monomeric glucose units are selected or isolated. In various instances, the number of monomeric glucose units in a β-1,6-glucan oligomer is provided or determined after conjugation with cetuximab or a cetuximab antibody for example by mass spectrometry (e.g., MALDI).

### Glucan Load

In some embodiments, a cetuximab or cetuximab antibody molecule present in a conjugate of the present invention may be conjugated to one or more β-1,6-glucan oligomers. In certain embodiments, it is conjugated to between 1 and 6 β-1,6-glucan oligomers (e.g., between 1 and 5, 1 and 4 or 1 and 3 β-1,6-glucan oligomers, e.g., 1,2, 3, 4, 5, or 6 β-1,6-glucan oligomers). In certain embodiments, it is conjugated to between 2 and 4 β-1,6-glucan oligomers. In certain embodiments, it is conjugated to 2 or 3 β-1,6-glucan oligomers. In certain embodiments, it is conjugated to 3 or 4 β-1,6-glucan oligomers. In certain embodiments, it is conjugated to 3 β-1,6-glucan oligomers. It is to be understood that when two or more β-1,6-glucan oligomers are conjugated to the same cetuximab or cetuximab antibody molecule, the two or more β-1,6-glucan oligomers may have the same or different lengths. In some embodiments, the two or more β-1,6-glucan oligomers have the same length.

As used herein, the term “glucan load” refers to the actual or average number of individual β-1,6-glucan oligomers that are conjugated to each cetuximab or cetuximab antibody molecule. A glucan load may be a whole number, e.g., when referring to the load of a single conjugate or a population of conjugates each having the same load. A glucan load may also be a whole number when referring to the load of a population of conjugates wherein the whole number load is representative of the actual load found on at least 90% of conjugates in the population (e.g., at least 91%, 92%, 93%, 94%, 95%, 96%, 97%, 98%, 99%, or 100% of molecules in the population). A glucan load may be presented as a fraction when a population includes a mixture of conjugates having two or more different loads. The fraction may be indicative of a hypothetical, expected, approximate, or measured average load of conjugates in the population. Accordingly, a glucan load expressed as being between two whole numbers encompasses any intervening fraction of a whole number.

Various methods of determining load of β-1,6-glucan oligomers are known in the art. In various instances, a conjugate is synthesized to have a particular load. In various instances a conjugate or population of conjugates each having a particular load is provided and the load is determined by chromatography (e.g., size exclusion chromatograph) and/or mass spectrometry (e.g., MALDI) and/or SDS-PAGE. In various instances, a population of conjugates having various loads is provided and load is determined by chromatography (e.g., size exclusion chromatograph) and/or mass spectrometry (e.g., MALDI) and/or SDS-PAGE. In various instances, conjugates having a particular load are selected or isolated.

### Conjugation

In various embodiments of the present invention, one or more of the aforementioned β-1,6-glucan oligomers are conjugated as described herein to cetuximab or a cetuximab antibody. In particular embodiments, one or more β-1,6-glucan oligomers are conjugated via a linker. In some embodiments, the one or more β-1,6-glucan oligomers are each independently conjugated to cetuximab or a cetuximab antibody, e.g., via a lysine residue.

In some embodiments, a conjugate of the present invention is of the Formula II:

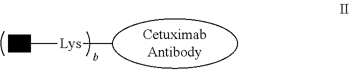

wherein:

Lys is a lysine residue;

b is between 1 and 6, 1 and 5, 1 and 4 or 1 and 3; and

[symbol] is a compound of Formula I or Formula Ia.

Formula II is intended to be a schematic illustration of the conjugation of a compound of Formula I or Formula Ia to a cetuximab antibody. Accordingly, when b is 1, and [symbol] is a compound of Formula Ia, the conjugate of Formula II can be drawn as:

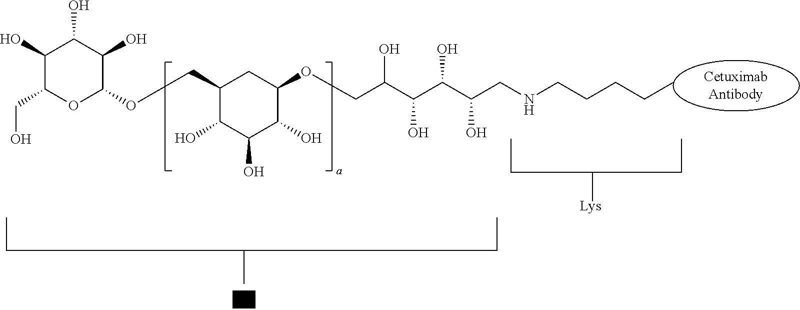

wherein “Lys” illustrates the aliphatic chain and terminal amine portion of the lysine residue.

Additionally, the structures above are intended to illustrate that multiple instances of the compound of Formula I or Formula Ia can be part of a conjugate. For example, when b is 2 in Formula II above, the conjugate is represented by the following schematic:

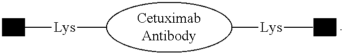

and when b is 3 in Formula II above, the conjugate is represented by the following schematic:

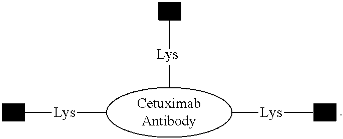

### Exemplary Conjugates

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of between 2 and 10 glucose monomer units;

a cetuximab antibody (e.g., cetuximab) conjugated to between 1 and 5 β-1,6-glucan oligomers, wherein each β-1,6-glucan oligomer is independently comprised of between 2 and 10 glucose monomer units;

a cetuximab antibody (e.g., cetuximab) conjugated to between 1 and 4 β-1,6-glucan oligomers, wherein each β-1,6-glucan oligomer is independently comprised of between 2 and 10 glucose monomer units;

a cetuximab antibody (e.g., cetuximab) conjugated to between 1 and 3 β-1,6-glucan oligomers, wherein each β-1,6-glucan oligomer is independently comprised of between 2 and 10 glucose monomer units;

a cetuximab antibody (e.g., cetuximab) conjugated to between 2 and 4 β-1,6-glucan oligomers, wherein each β-1,6-glucan oligomer is independently comprised of between 2 and 10 glucose monomer units;

a cetuximab antibody (e.g., cetuximab) conjugated to between 2.5 and 3.5 β-1,6-glucan oligomers, wherein each β-1,6-glucan oligomer is independently comprised of between 2 and 10 glucose monomer units; or

a cetuximab antibody (e.g., cetuximab) conjugated to 3 β-1,6-glucan oligomers, wherein each β-1,6-glucan oligomer is independently comprised of between 2 and 10 glucose monomer units.

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of between 3 and 7 glucose monomer units;

a cetuximab antibody (e.g., cetuximab) conjugated to between 1 and 5 β-1,6-glucan oligomers, wherein each β-1,6-glucan oligomer is independently comprised of between 3 and 7 glucose monomer units;

a cetuximab antibody (e.g., cetuximab) conjugated to between 2 and 4 β-1,6-glucan oligomers, wherein each β-1,6-glucan oligomer is independently comprised of between 3 and 7 glucose monomer units;

a cetuximab antibody (e.g., cetuximab) conjugated to between 2.5 and 3.5 β-1,6-glucan oligomers, wherein each β-1,6-glucan oligomer is independently comprised of between 3 and 7 glucose monomer units; or

a cetuximab antibody (e.g., cetuximab) conjugated to 3 β-1,6-glucan oligomers, wherein each β-1,6-glucan oligomer is independently comprised of between 3 and 7 glucose monomer units.

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of between 3 and 5 glucose monomer units;

a cetuximab antibody (e.g., cetuximab) conjugated to between 1 and 5 β-1,6-glucan oligomers, wherein each β-1,6-glucan oligomer is independently comprised of between 3 and 5 glucose monomer units;

a cetuximab antibody (e.g., cetuximab) conjugated to between 2 and 4 β-1,6-glucan oligomers, wherein each β-1,6-glucan oligomer is independently comprised of between 3 and 5 glucose monomer units;

a cetuximab antibody (e.g., cetuximab) conjugated to between 2.5 and 3.5 β-1,6-glucan oligomers, wherein each β-1,6-glucan oligomer is independently comprised of between 3 and 5 glucose monomer units; or

a cetuximab antibody (e.g., cetuximab) conjugated to 3 β-1,6-glucan oligomers, wherein each β-1,6-glucan oligomer is independently comprised of between 3 and 5 glucose monomer units.

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of between 3.5 and 4.5 glucose monomer units;

a cetuximab antibody (e.g., cetuximab) conjugated to between 1 and 5 β-1,6-glucan oligomers, wherein each β-1,6-glucan oligomer is independently comprised of between 3.5 and 4.5 glucose monomer units;

a cetuximab antibody (e.g., cetuximab) conjugated to between 2 and 4 β-1,6-glucan oligomers, wherein each β-1,6-glucan oligomer is independently comprised of between 3.5 and 4.5 glucose monomer units;

a cetuximab antibody (e.g., cetuximab) conjugated to between 2.5 and 3.5 β-1,6-glucan oligomers, wherein each β-1,6-glucan oligomer is independently comprised of between 3.5 and 4.5 glucose monomer units; or

a cetuximab antibody (e.g., cetuximab) conjugated to 3 β-1,6-glucan oligomers, wherein each β-1,6-glucan oligomer is independently comprised of between 3.5 and 4.5 glucose monomer units.

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of 4 glucose monomer units;

a cetuximab antibody (e.g., cetuximab) conjugated to between 1 and 5 β-1,6-glucan oligomers, wherein each β-1,6-glucan oligomer is independently comprised of 4 glucose monomer units;

a cetuximab antibody (e.g., cetuximab) conjugated to between 2 and 4 β-1,6-glucan oligomers, wherein each β-1,6-glucan oligomer is independently comprised of 4 glucose monomer units;

a cetuximab antibody (e.g., cetuximab) conjugated to between 2.5 and 3.5 β-1,6-glucan oligomers, wherein each β-1,6-glucan oligomer is independently comprised of 4 glucose monomer units; or

a cetuximab antibody (e.g., cetuximab) conjugated to 3 β-1,6-glucan oligomers, wherein each β-1,6-glucan oligomer is independently comprised of 4 glucose monomer units.

In some exemplary embodiments of the present invention, a conjugate may have (or a composition may comprise conjugates having) the following features:

a cetuximab antibody (e.g., cetuximab) conjugated to between 1 and 6 β-1,6-glucan oligomers, wherein each β-1,6-glucan oligomer is independently comprised of 5 glucose monomer units;

a cetuximab antibody (e.g., cetuximab) conjugated to between 1 and 5 β-1,6-glucan oligomers, wherein each β-1,6-glucan oligomer is independently comprised of 5 glucose monomer units;

a cetuximab antibody (e.g., cetuximab) conjugated to between 4 and 6 β-1,6-glucan oligomers, wherein each β-1,6-glucan oligomer is independently comprised of 5 glucose monomer units;

a cetuximab antibody (e.g., cetuximab) conjugated to 5 β-1,6-glucan oligomers, wherein each β-1,6-glucan oligomer is independently comprised of 5 glucose monomer units;

a cetuximab antibody (e.g., cetuximab) conjugated to between 2 and 4 β-1,6-glucan oligomers, wherein each β-1,6-glucan oligomer is independently comprised of 5 glucose monomer units; or

a cetuximab antibody (e.g., cetuximab) conjugated to between 2 and 3 β-1,6-glucan oligomers, wherein each β-1,6-glucan oligomer is independently comprised of 5 glucose monomer units.

In some exemplary embodiments of the present invention, a conjugate may be represented by Formula IIa:

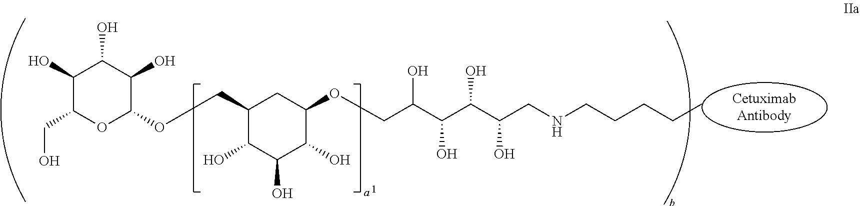

wherein a¹ is between 1 and 9; and b is between 1 and 6. In other embodiments a¹ is 1, 2, 3, 4, 5, 6, 7, 8 or 9. In other embodiments, b is 1, 2, 3, 4, 5, or 6. In another exemplary embodiment, the conjugate is represented by Formula IIa, wherein a¹ is between 1 and 9, and b is between 2 and 4.

In another exemplary embodiment, the conjugate is represented by Formula IIa, wherein a¹ is between 1 and 9, and b is 3.

In another embodiment, the conjugate is represented by Formula IIa, wherein a¹ is between 1 and 3, and b is between 1 and 6. In other embodiments, a is 1, 2, or 3. In other embodiments, b is 1, 2, 3, 4, 5, or 6. In another exemplary embodiment, the conjugate is represented by Formula IIa, wherein a¹ is between 1 and 3, and b is between 2 and 4.

In another exemplary embodiment, the conjugate is represented by Formula IIa, wherein a¹ is between 1 and 3, and b is 3.

In another embodiment, the conjugate is represented by Formula IIa, wherein a¹ is between 2 and 4, and b is between 1 and 6. In other embodiments, a¹ is 2, 3, or 4. In other embodiments, b is 1, 2, 3, 4, 5, or 6. In another exemplary embodiment, the conjugate is represented by Formula IIa, wherein a¹ is between 2 and 4, and b is between 2 and 4.

In another exemplary embodiment, the conjugate is represented by Formula IIa, wherein a¹ is between 2 and 4, and b is 3.

In another embodiment, the conjugate is represented by Formula IIa, wherein a¹ is 3, and b is between 1 and 6. In other embodiments, b is 1, 2, 3, 4, 5, 6. In another exemplary embodiment, the conjugate is represented by Formula IIa, wherein a¹ is 3, and b is between 2 and 4. In another exemplary embodiment, the conjugate is represented by Formula IIa, wherein a¹ is 3, and b is 3.

In another embodiment, the conjugate is represented by Formula IIa, wherein a¹ is 4, and b is between 1 and 6. In other embodiments, b is 1, 2, 3, 4, 5, 6. In another exemplary embodiment, the conjugate is represented by Formula IIa, wherein a¹ is 4, and b is between 2 and 4 or between 4 and 6. In another exemplary embodiment, the conjugate is represented by Formula IIa, wherein a¹ is 4, and b is 3. In another exemplary embodiment, the conjugate is represented by Formula IIa, wherein a¹ is 4, and b is 5.

### Conjugate Properties

In any of the various embodiments described herein, a cetuximab antibody conjugate of the present invention may be capable of binding EGFR, e.g., EGFR expressed by a cancer or tumor cell. In some embodiments, a cetuximab antibody conjugate of the present invention competes with cetuximab for binding with EGFR. In some embodiments, conjugation enhances complement (C3) deposition. C3 deposition can be assayed by any known method, including Western analysis or FACS analysis using monoclonal antibodies directed against the alpha or beta chains of C3. In some embodiments, conjugation enhances binding by anti-β-1,6-glucan antibodies. Binding by anti-β-1,6-glucan antibodies can be assayed by any known method, including ELISA analysis using anti-human IgG2 antibodies. In these various embodiments, the enhancement as compared to an unconjugated counterpart may be at least 5%, at least 10%, at least 20%, at least 30%, at least 40%, at least 50%, at least 75%, at least 100%, or more.

In some embodiments, a conjugate of the present invention may exhibit one or more or all of these properties and/or one or more or all of the following properties: an EC₅₀ in an ADCC assay which is substantially the same as its unconjugated counterpart (e.g., cetuximab or cetuximab antibody); an AUC in a mouse PK assay in absence of tumor which is substantially the same as its unconjugated counterpart (e.g., cetuximab or cetuximab antibody); a PK profile which is substantially the same in the presence or absence of anti-glucan antibodies; a half-life in a mouse PK assay in presence of tumor which is substantially the same as its unconjugated counterpart (e.g., cetuximab or cetuximab antibody); a half-life in a mouse PK assay in absence of tumor which is substantially the same as its unconjugated counterpart (e.g., cetuximab or cetuximab antibody); a half-life in a mouse PK assay in presence of tumor which is substantially shorter than that of its unconjugated counterpart (e.g., cetuximab or cetuximab antibody); or a half-life in a mouse PK assay in absence of tumor which is substantially shorter than that of its unconjugated counterpart (e.g., cetuximab or cetuximab antibody). In these various embodiments, a conjugate produces a result which is “substantially the same” as its unconjugated counterpart if the result is within 20%, within 10%, within 5%, within 4%, within 3%, within 2%, within 1%, or the same as, the result obtained with the unconjugated counterpart. In various embodiments, a conjugate half-life is “substantially shorter” as compared to its unconjugated counterpart if the half-life of the conjugate is less than 80% of the half-life of the unconjugated counterpart, e.g., less than 80%, less than 70%, less than 60%, less than 50%, less than 40%, or less than 30% of the half-life of the unconjugated counterpart. In various embodiments, a conjugate as described herein is active in vivo.

### Uses and Administration

The following uses, and methods apply to any conjugate described herein. A cetuximab antibody conjugate of the present invention may be used, e.g., in the treatment of cancer, e.g., a cancer associated with expression of EGFR. A conjugate of the present invention may be used, e.g., in the treatment of colorectal cancer (e.g., KRAS wild-type EGFR-expressing colorectal cancer), squamous cell carcinoma, e.g., of the head or neck (SCCHN), and/or other EGFR overexpressing tumors such as triple negative breast cancer and lung cancer. In some embodiments, the cancer is metastatic. In various embodiments, a cancer or tumor treated by administration of a conjugate described herein is a recurrent or treatment-resistant cancer or tumor (e.g., a cancer or tumor resistant to treatment by unconjugated cetuximab). Such treatment may be, e.g., in a subject having, suspected of having, or diagnosed as having such cancer. In some embodiments, a cetuximab antibody conjugate of the present invention may be used to treat a subject having a KRAS mutant cancer (e.g., KRAS mutant EGFR-expressing colorectal cancer). One non-limiting example of a KRAS mutation is one which leads to a G13D substitution. In some embodiments, a cetuximab antibody conjugate of the present invention may be used to treat a subject having a BRAF mutant cancer (e.g., BRAF mutant EGFR-expressing colorectal cancer). One non-limiting example of a BRAF mutation is one which leads to a V599E substitution.

In various embodiments, a conjugate described herein, upon administration to a subject having, suspected of having, or diagnosed as having a cancer or tumor, is cytostatic, cytotoxic, or slows, delays, or inhibits growth of the cancer or tumor. In various embodiments, a conjugate described herein, upon administration to a subject having, suspected of having, or diagnosed as having a cancer or tumor, increases the length or likelihood survival of the subject. In various embodiments, a conjugate described herein, upon administration to a subject having, suspected of having, or diagnosed as having a cancer or tumor, induces regression or stasis of a cancer or tumor. In various embodiments, a conjugate described herein, upon administration to a subject having, suspected of having, or diagnosed as having a cancer or tumor, induces an immune response that is effective in inhibiting recurrence of a cancer or tumor. In various embodiments, a conjugate described herein recruits neutrophils, e.g., to a targeted cancer or tumor. In various embodiments, a conjugate described herein causes or promotes neutrophil infiltration, e.g., of a targeted cancer or tumor. In various embodiments, administration of a conjugate described herein to a subject having, suspected of having, or diagnosed as having a cancer or tumor does not elicit an adverse effect, e.g., a cytokine storm or sepsis.

In some embodiments, a conjugate of the present invention produces an enhanced therapeutic response in a subject with a tumor as compared to its unconjugated counterpart (i.e., cetuximab or cetuximab antibody). For example, and in some embodiments, conjugation enhances phagocytosis and/or cytotoxic responses to tumor cells, or in some embodiments, enhances complement-mediated lysis of the tumor cells. In some embodiments, these responses are mediated by neutrophils and/or macrophages. Phagocytosis and/or lysis can be assessed by any known method, including time-lapse microscopy or Fluorescence-Activated Cell Sorting (FACS). In these various embodiments, the enhancement as compared to an unconjugated counterpart may be at least 5%, at least 10%, at least 20%, at least 30%, at least 40%, at least 50%, at least 75%, at least 100%, or more.

In some embodiments, a conjugate of the present invention may exhibit any of these properties and/or one or more of the following properties: a reduced rate of tumor growth when administered (e.g., twice weekly 5 mg/kg equivalent dose of cetuximab or cetuximab antibody) to a BRAF mutant colorectal tumor xenograft mouse model as compared to administration of its unconjugated counterpart (e.g., twice weekly 5 mg/kg dose of cetuximab or cetuximab antibody); a reduced rate of tumor growth when administered (twice weekly 5 mg/kg equivalent dose of cetuximab or cetuximab antibody) to a KRAS mutant colorectal tumor xenograft mouse model as compared to its unconjugated counterpart (e.g., twice weekly 5 mg/kg dose of cetuximab or cetuximab antibody); tumor regression or statis when administered (e.g., twice weekly 5 mg/kg equivalent dose of cetuximab or cetuximab antibody for 30 days) to a KRAS mutant colorectal tumor xenograft mouse model; or increased survival post-treatment when administered (e.g., twice weekly 5 mg/kg equivalent dose of cetuximab or cetuximab antibody for 30 days) to a KRAS mutant colorectal tumor xenograft mouse model as compared to its unconjugated counterpart (e.g., twice weekly 5 mg/kg dose of cetuximab or cetuximab antibody for 30 days). In various embodiments, administration of a conjugate of the present invention may elicit an adaptive T cell response in a subject (e.g., a human or rodent), which adaptive T cell response may be elicited, e.g., directly or through activation of an innate immune response (e.g., neutrophils or macrophages).

In various embodiments, conjugates described herein can be incorporated into a pharmaceutical composition. Such a pharmaceutical composition can be useful, e.g., for the treatment of a cancer or tumor, e.g., a cancer or tumor described herein. Pharmaceutical compositions of the present invention can be formulated by methods known to those skilled in the art (e.g., as described in Remington: The Science and Practice of Pharmacy, 22nd edition, ed. Lloyd Allen, Pharmaceutical Press and Philadelphia College of Pharmacy at University of the Sciences, 2012, the contents of which are incorporated herein by reference).

A pharmaceutical composition can include a therapeutically effective amount of a conjugate described herein. Such effective amounts can be readily determined by one of ordinary skill in the art based, in part, on the effect of the administered composition, or the combinatorial effect of the conjugate and one or more additional active agents, if more than one agent is used. A therapeutically effective amount of a conjugate described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of the disease or disorder. Other factors affecting the dose administered to the subject include, e.g., the type or severity of the disease or disorder. Other factors can include, e.g., other medical disorders concurrently or previously affecting the subject, the general health of the subject, the genetic disposition of the subject, diet, time of administration, rate of excretion, etc. Suitable human doses of any of the compositions described herein can further be evaluated in, e.g., Phase I dose escalation studies.

The route of administration can be parenteral, for example, administration by injection. A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, and other suitable excipients followed by mixing in a unit dose form required for generally accepted pharmaceutical practices. The amount of conjugate included in the pharmaceutical compositions is such that a suitable dose within the designated range is provided. The formulated injection can be packaged in a suitable ampule.

### EXAMPLES

The examples described herein demonstrate, among other things, the production and characterization of various mAbXcite-cetuximab conjugates (i.e., cetuximab linked to one or more β-1,6-glucan oligomers). Conjugates constructed with different conjugation chemistries were studied, as were the effect of various β-1,6-glucan oligomer lengths and loads. For instance, described herein are results from the study of different conjugation chemistries including click chemistry (DBCO) and reductive amination (also called “direct conjugation” herein). Also described herein are studies relating to the effect of conjugation with β-1,6-glucan oligomers of different length, e.g., from 4 to 9 glucose monomer units, and different loads, e.g., averaging from 1 to 6 β-1,6-glucan oligomers per cetuximab molecule. Binding of cetuximab to EGFR when conjugated to β-1,6-glucan oligomers as well as its antibody-dependent cell-mediated cytotoxicity (ADCC) was tested and found to be similar to unconjugated cetuximab. Among results described in the present Examples, mAbXcite-cetuximab conjugates were confirmed to recruit neutrophils by a live imaging technique and histology. Results described herein also include successful preclinical results in tumor xenograft mouse models with BRAF and KRAS mutations.

In certain of the present Examples, mice were selected as a useful model for testing β-1,6-glucan conjugates, as these mammals have only low titers of endogenous anti-β-1,6-glucan. Levels of anti-β-1,6-glucan antibodies could therefore be controlled via administration of IVIG.

As used in the following Examples, the glucans are sometimes referred to as “#-mers” wherein “#” is the number of glucose monomer units that were present prior to conjugation with cetuximab. For example, a glucan including three glucose monomer units prior to conjugation may be identified as a “3-mer” or “3mer”, a glucan including four glucose monomer units may be identified as a “4-mer” or “4mer”, a glucan including five glucose monomer units may be identified as a “5-mer” or “5mer”, and so forth without limitation. When glucans are conjugated via reductive amination (direct conjugation) one of the glucose monomer units is ring-opened to form a “linker” between the remaining glucose monomer units and cetuximab. Accordingly, references to conjugates that were prepared by direct conjugation using a “3-mer” would include the following structure:

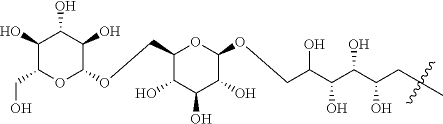

and when a non-glucose based linker such as, for example, NHS DBCO is used with, e.g., a “4-mer”, the resulting conjugate would include the following structure:

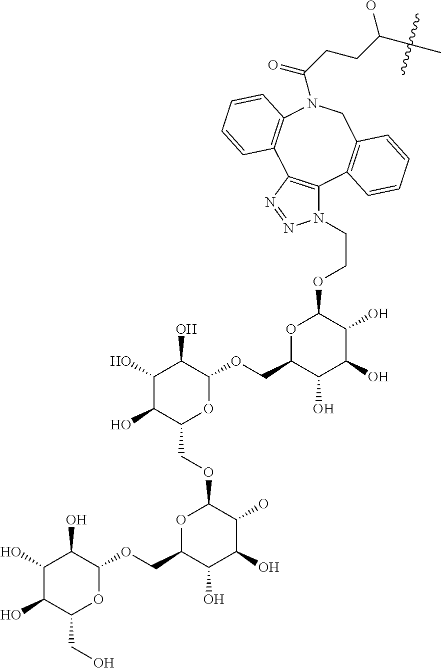 [SMILES: *C(O)CCC(=O)N1Cc2ccccc2-c2c(nnn2CCO[C@@H]2O[C@H](CO[C@@H]3O[C@H](CO[C@@H]4O[C@H](CO[C@@H]5O[C@H](CO)[C@@H](O)[C@H](O)[C@H]5O)[C@@H](O)[C@H](O)[C@H]4O)[C@@H](O)[C@H](O)[C@H]3O)[C@@H](O)[C@H](O)[C@H]2O)-c2ccccc21]

Although exemplary embodiments are described in detail in these Examples, it should be understood that various changes, substitutions and alterations can be made herein without departing from the spirit and scope of the invention as defined in the appended claims.

### Example 1: Conjugation of Cetuximab to Glucan does not Change Binding to EGFR

Studies relating to whether conjugation affects cetuximab binding to EGFR were undertaken. These experiments were performed using cetuximab and rituximab as controls. Samples were detected using polyclonal human anti-β-1,6-glucan antibodies and mouse anti-human IgG-FITC. Also utilized were a blocking and staining buffer (PBS pH 7.2+2% BSA) and a washing buffer (PBS pH 7.2).

2×10⁵ A431 (Epithelial cells, Epidermoid carcinoma) cells were blocked for 1 hour at room temperature in PBS-BSA, followed by an incubation with mAbXcite-cetuximab, cetuximab or rituximab (negative control) in staining buffer for 1 hour at RT. Cells were then washed (×3), and a detection antibody (mouse anti-human IgG-FITC) was added at 1:100 dilution. After three more washes, cells were spun down, re-suspended in PBS and analyzed by FACS using FACSCalibur (BD Biosciences). The cells were analyzed by FACS (FL1 for FITC).

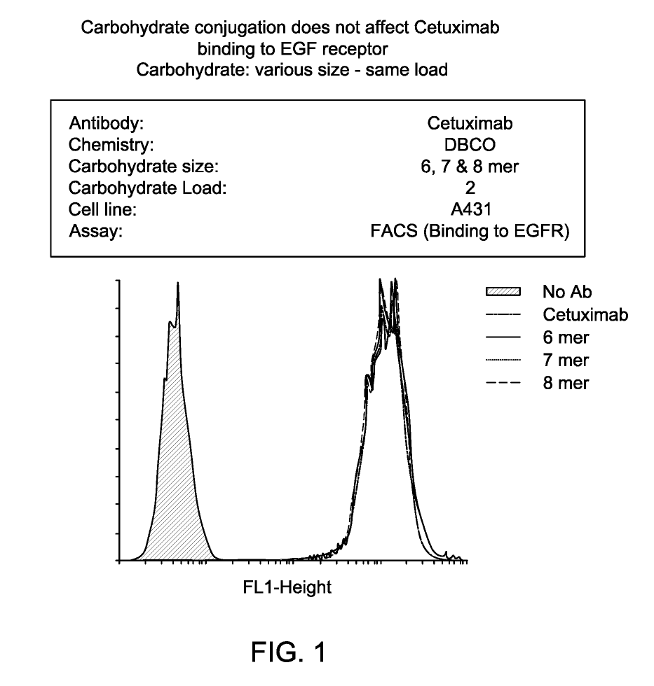

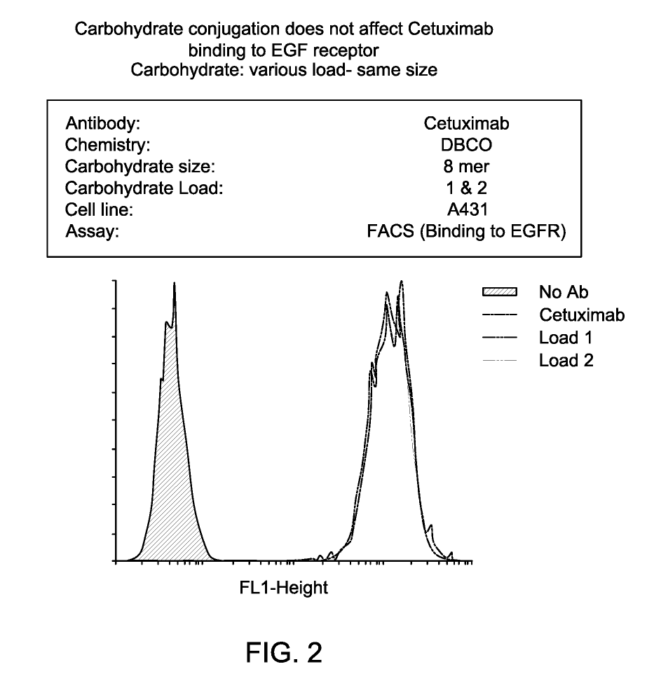

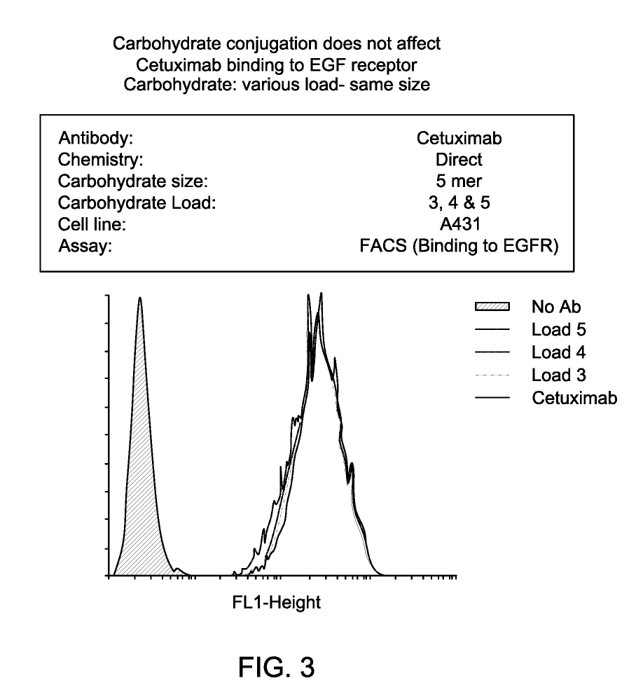

β-1,6-glucan conjugation did not impair cetuximab binding to EGFR. mAbXcite-cetuximab binding to EGFR was assessed by FACS using rituximab as a non-specific control (“No Ab”), cetuximab and mAbXcite-cetuximab (6-mer, 7-mer, or 8-mer β-1,6-glucan oligomers), and for loads ranging from 1 to 5 β-1,6-glucan oligomers per antibody (FIGS. 1, 2, and 3). The results show that none of the conjugates or loads (in particular 1 or 2 load of 8-mer β-1,6-glucan oligomers or 3 to 5 load of 5-mer β-1,6-glucan oligomers) were impaired for binding to EGFR as compared to a cetuximab control. In this and other Examples discussed herein that involved direct conjugation, β-1,6-glucan oligomers were purified from pustulan. As described in Example 26, pustulan was treated to yield a ladder of β-1,6-glucans from which oligomers having the desired length were prepared, e.g., by fractionation and purification.

### Example 2: Conjugation of Cetuximab to Glucan does not Change ADCC

We also studied whether conjugation with glucan affects cetuximab antibody-dependent cell-mediated cytotoxicity (ADCC). In this assay, the readout was luminescence signal from expression of firefly luciferase driven by an NFAT response element.

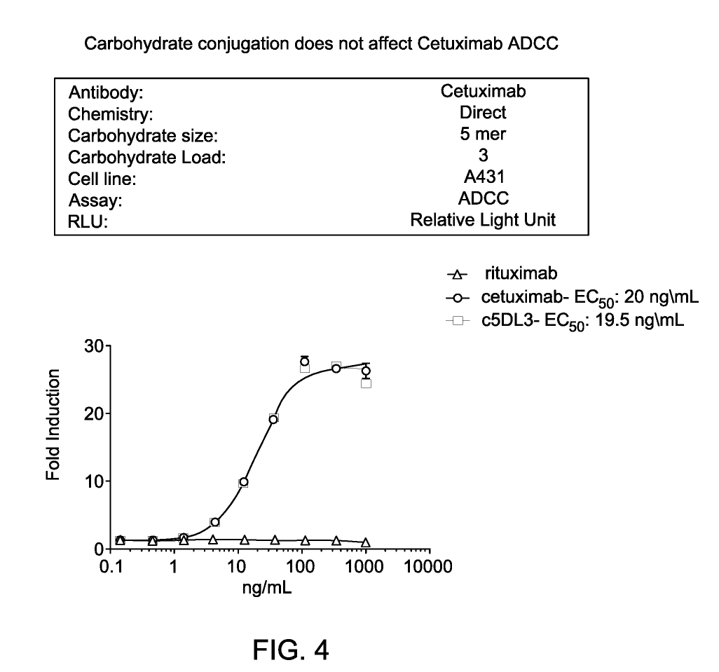

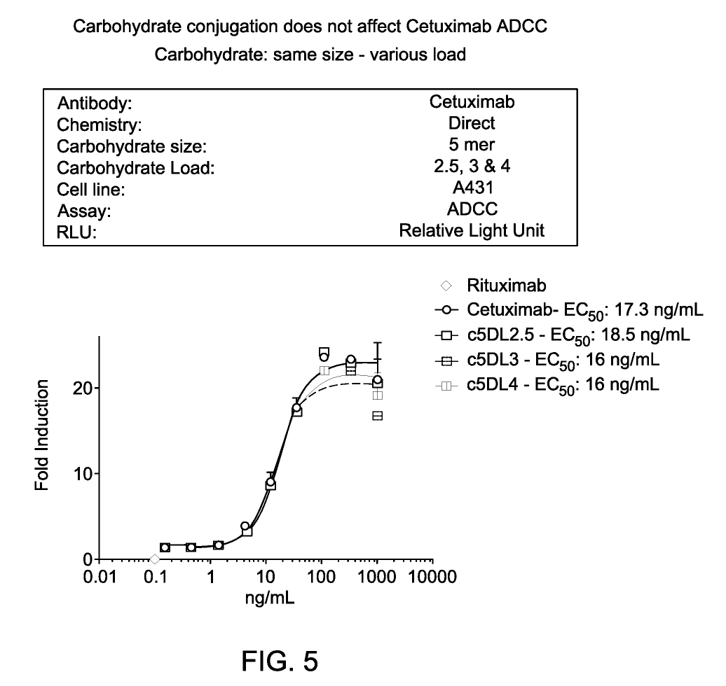

These experiments were also performed using cetuximab and rituximab as controls. ADCC Reporter Bioassay response to mAbXcite-cetuximab was studied using ADCC Bioassay Effector human and epithelial carcinoma cell line A431 as target cells. Experiments were performed using an ADCC Reporter Bioassay (Promega). Briefly, target cells (A431) were plated at the density of 5,000 cells per well in complete culture medium overnight. On the day of bioassay, antibodies (cetuximab, cetuximab-mAbXcite and rituximab as a control) were serially diluted. After carefully removing the cell medium, conjugate and antibodies, at various concentrations, were added to the cells, followed by addition of ADCC Bioassay Effector Cells. The E:T ratio was 15:1. After 6 hours of induction at 37° C., Bio-Glo™ Luciferase Assay Reagent was added and luminescence determined using an Envision luminometer (FIGS. 4 and 5).

The data were fitted to a 4PL curve using GraphPad Prism® software. The EC₅₀ of response using cetuximab/A431 target cells was around 20 ng/ml. The results demonstrate that β-1,6-glucan conjugation did not change cetuximab ADCC (mAbXcite-cetuximab having a 2.5, 3, or 4 load of 5-mer oligomers) as compared to cetuximab control in an ADCC Reporter Bioassay using ADCC Bioassay Effector Cells and A431 Target Cells.

### Example 3: β-1,6-Glucan Oligomer Length and Load

Two main potency assays were developed to assess antibody activity in vitro.

In the first such assay, antibody activity was assessed using an anti-β-1,6-glucan ELISA. In this ELISA, recombinant EGFR peptide was absorbed to an ELISA plate. Following the binding of cetuximab or mAbXcite-cetuximab the β-1,6-oligomer was detected by anti-β-1,6-glucan antibodies. The results of this assay correlate well with the average oligomer load as assayed by MALDI-TOF.

In the second such assay, antibody activity was assessed using anti-β-1,6-glucan FACS. In this assay the antibodies were added to live cells rather than to an EGFR peptide. The β-1,6-oligomer was detected by anti-β-1,6-glucan antibodies. The results of this assay also correlate well with the average oligomer load as assayed by MALDI-TOF.

### ELISA Assay

The present studies were undertaken to evaluate the binding of anti-β-1,6-glucan antibodies to mAbXcite-cetuximab. In the present experiment, in order to assess the impact of β-1,6-glucan oligomer length on mAbXcite-cetuximab activity, the oligomer load was 2 or 6, oligomer length was 4-mer to 9-mer, and the linkage of glucan to cetuximab was either DBCO or direct. Cetuximab was used as a control. Also utilized were a blocking and staining buffer (PBS pH 7.2+2% BSA) and a PBS-Tween buffer (PH 7.2, 0.05% Tween). ELISA was carried out using 1-Step™ Ultra TMB-ELISA (Pierce).

A recombinant EGFR peptide (rhEGFR/Fc Chimera peptide from R&D) was absorbed on an ELISA plate (MaxiSorp) overnight at 4° C. After 3 washes, non-specific binding sites were blocked for 1 hour at room temperature. Cetuximab or mAbXcite-cetuximab were then added and incubation performed on a plate shaker for 1 hour at room temperature, followed by 3 additional washes. To allow detection of the β-1,6-glucan oligomer, anti-β-1,6-glucan antibodies (polyclonal human anti-β-1,6-glucan antibodies) were added to the plate (3 ug/well) and incubated for 45 min at RT. The wells were washed 3 times and incubated with a 1:5000 dilution of anti-human IgG2 (Fc)-HRP antibody (mouse anti-human IgG2 (Fc)-HRP) for 45 min at RT. Extensive washes were performed (×5) and visualization was carried out with 100 uL of TMB substrate.

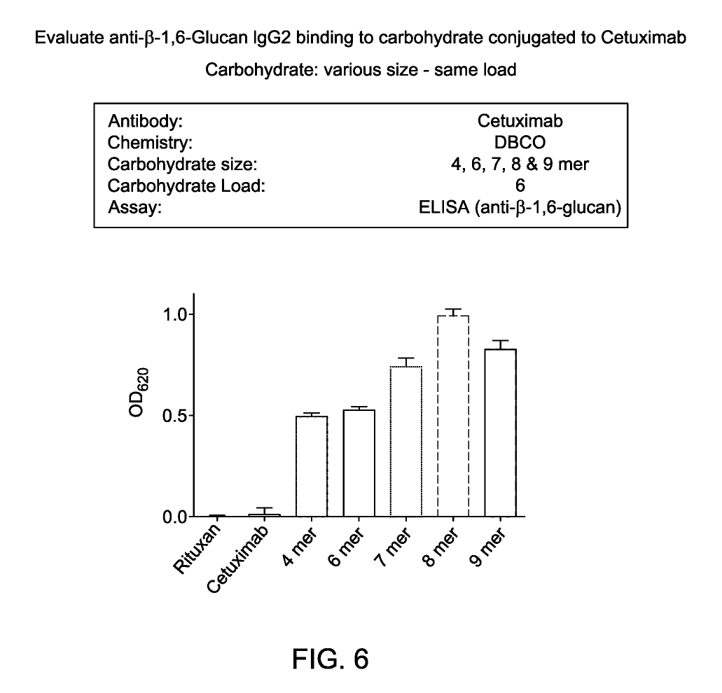

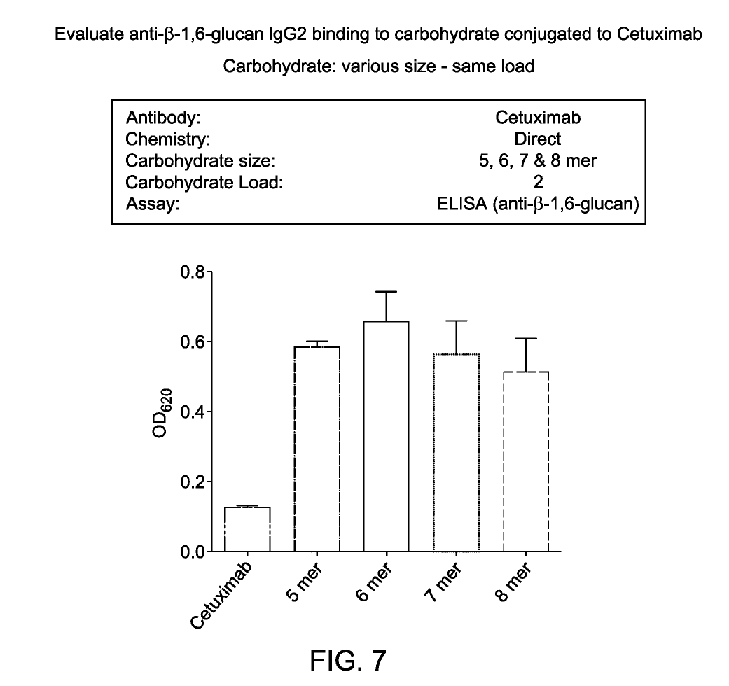

The plate was read immediately at OD620 nm—and reading occurred every minute for one hour. Data shown are the mean+/−standard deviation of 3 replicate wells (FIGS. 6 and 7). Binding to DBCO (A) and direct (B) conjugates was evaluated by ELISA. All mAbXcite-cetuximab conjugates tested were active in this assay. An increase in activity was observed with increases in oligomer length (up to 7-8-mer) where saturation was reached.

### FACS Assay

The present studies were undertaken to identify whether all β-1,6-glucan oligomer lengths are recognized by anti-β-1,6-glucan antibodies when mAbXcite-cetuximab is bound to EGFR on live cells. Data was acquired using the conjugate mAbXcite-cetuximab with cetuximab as a control. In the present experiment, the β-1,6-glucan oligomer load was 1 to 6, the oligomer length ranges from 4-mer to 9-mer, and the linkage of glucan to cetuximab was DBCO.

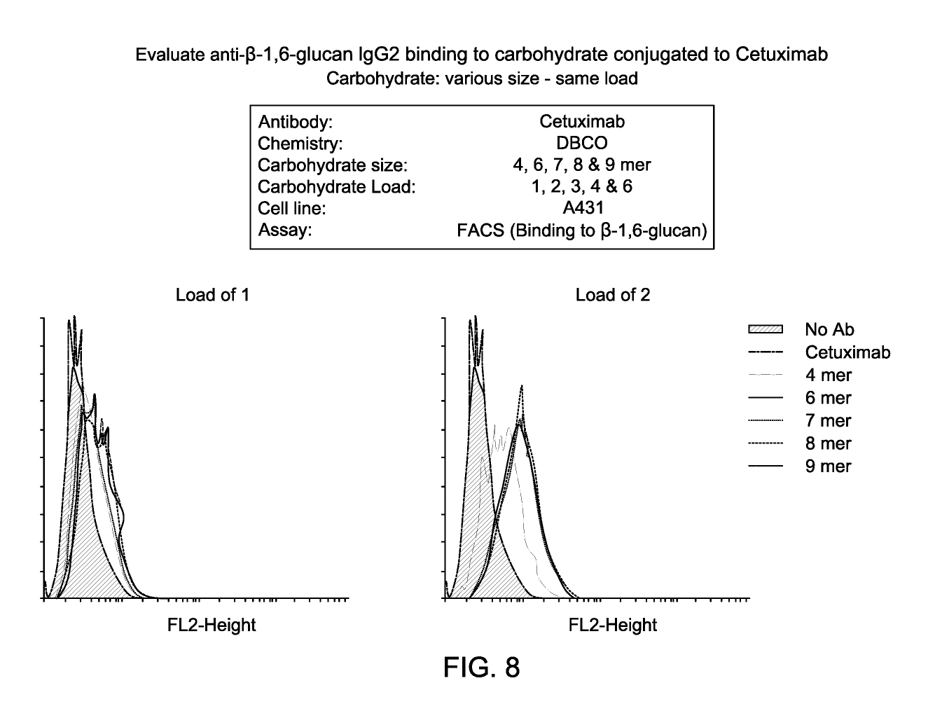

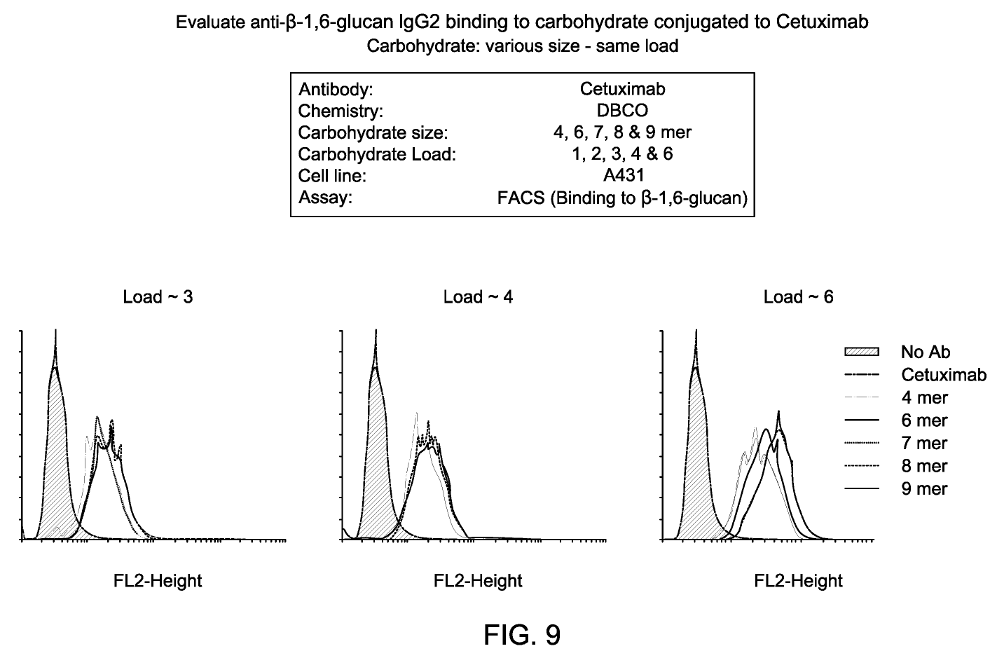

2×10⁵ A431 (Epithelial cells, Epidermoid carcinoma) cells were blocked for 1 hour at room temperature in PBS-BSA (PBS pH 7.2+2% BSA) followed by an incubation with mAbXcite-cetuximab and cetuximab or rituximab (negative control) in staining buffer for 1 hour at RT. After 3 washes (PBS pH 7.2), cells were incubated with purified anti-β-1,6-glucan antibodies (polyclonal human anti-β-1,6-glucan antibodies) for 1 hour at RT. Cell were then washed (×3), and a detection antibody (mouse anti-human IgG2-PE) was added at 1:100 dilution. After three more washes, cells were spun down, re-suspended in PBS and analyzed by FACS. The cells were analyzed by FACS (FL2) using FACSCalibur (BD Biosciences). mAbXcite-cetuximab binding was assessed by FACS using rituximab as a non-specific control (“No Ab”), cetuximab and mAbXcite-cetuximab (4-mer to 9-mer). Results are shown in FIGS. 8 and 9. All mAbXcite-cetuximab analyzed in this section for binding to EGFR on A431 cells and for deposition of anti-β-1,6-glucan antibody were functional in vitro.

### Example 4: mAbXcite-Cetuximab Neutrophil Infiltration

The studies of this Example were undertaken to assess effect on pharmacodynamics (PD) in nude mice. Specifically, we evaluated the effect of conjugation with different oligomer lengths on neutrophil infiltration using 2 different approaches: live imaging using a luminol assay and histology using anti-neutrophil antibody.

### Live Imaging Assay

In this assay, luminol reacts with myeloperoxidase (which is a neutrophil-specific enzyme, and marker of activated neutrophils) and the resulting bioluminescence is detected by an IVIS imager. These experiments included mAbXcite-cetuximab with DBCO chemistry, 4-mer to 8-mer oligomer length, and a load of 2 oligomers per antibody. Cetuximab and pustulan (unconjugated β-1,6-glucan of undefined size) were used as a controls.

Mice were implanted SC with 2×10⁶ A431 (Epithelial cells, Epidermoid carcinoma) cells. When the tumors reached a volume of 300-400 mm³, which we have found to be ideal for imaging, mice were dosed with IVIG followed 2 hours later with mAbXcite-cetuximab, cetuximab, or vehicle control. 5 hours after treatment started, mice were anesthetized by isoflurane inhalation, administered with luminol (200 mg/kg IP) and imaged on IVIS 100 bioluminescence imaging system (Caliper Life sciences) within 7-15 minutes.

For quantification, total photon flux was calculated by the IVIS software and the background subtracted. All mice were imaged the day before treatment to assess the base line. After imaging, the tumors and blood were collected. The tumors were sent to histology (for correlation with imaging). Neutrophil activation was assessed by live imaging using PBS and cetuximab as negative controls, and pustulan (injected IT) as a positive control. A luminescent spot on the tumor indicates positives cells.

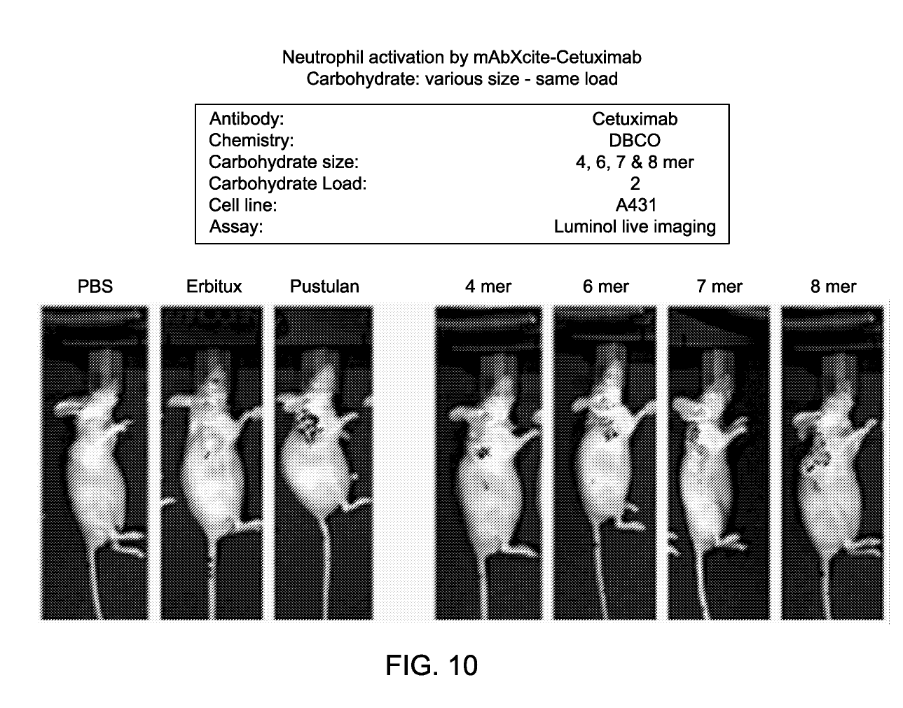

All mAbXcite-cetuximab constructs tested (with 4-mer to 8-mer oligomers) led to neutrophil activation in this assay. See FIG. 10.

### Histology Assay

These studies were undertaken to assess neutrophil infiltration in tumor upon mAbXcite-cetuximab treatment. The glucan conjugation used direct chemistry. 5-mer oligomers were used with a load of 3 oligomers per antibody.

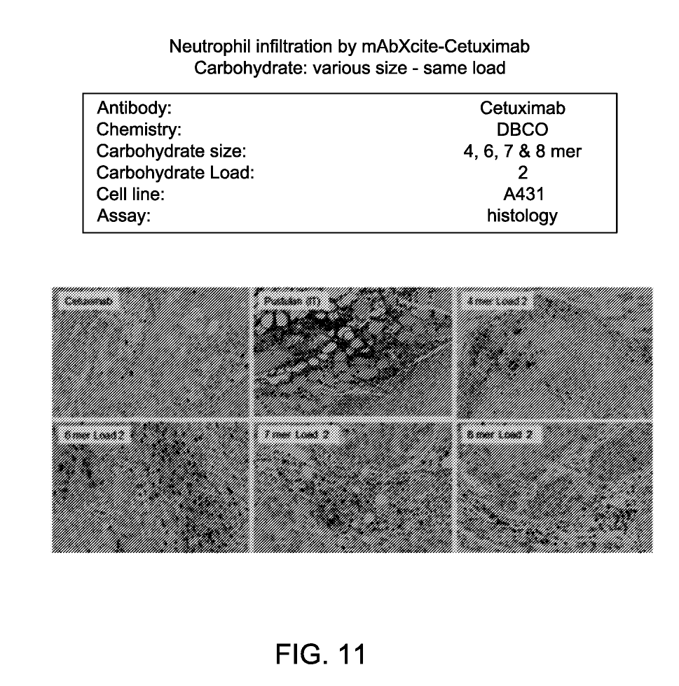

In this assay, mAbXcite-cetuximab was tested, with cetuximab and pustulan (unconjugated β-1,6-glucan of undefined size) as controls. Mice were implanted SC with 2×10⁶ A431 (Epithelial cells, Epidermoid carcinoma) cells. When the tumors reached a volume of 300-400 mm³, mice were dosed with IVIG followed 2 hours later with mAbXcite-cetuximab, cetuximab, or vehicle control. 5 hours after treatment started, tumor were collected and sent to histology for evaluating neutrophil infiltration. Tumors were fixed in formalin, embedded in paraffin blocks, and stained with a neutrophil-specific detection antibody (anti-neutrophil (Ly6G) antibody (Abcam No. 2557)). Neutrophil infiltration was assessed by immunohistochemistry (dark staining indicates positives cells). In correlation with the above live imaging study, neutrophil infiltration was also observed by histology upon treatment with all mAbXcite-cetuximab (with 4-mer to 8-mer oligomers) tested. See FIG. 11.

### Example 5: Binding of mAbXcite-Cetuximab Having Various Oligomer Loads with Anti-β-1,6-Glucan Antibody or EGFR

### ELISA Assay

ELISA was used to evaluate the binding of anti-β-1,6-glucan antibodies to mAbXcite-cetuximab, using cetuximab as a control. The present study utilized mAbXcite-cetuximab in which the conjugation utilized click chemistry (DBCO) using 6-mer oligomers at a load of 1-3 oligomers per antibody.

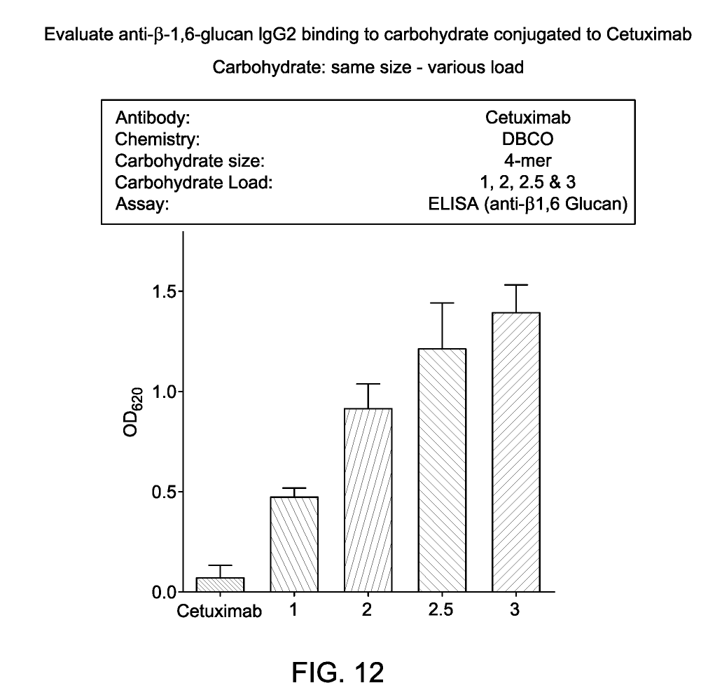

ELISA was carried out using 1-Step™ Ultra TMB-ELISA (Pierce). A human recombinant EGFR peptide (rhEGFR/Fc Chimera (peptide from R&D)) was absorbed on an ELISA plate (MaxiSorp) overnight at 4° C. Following a couple of washes, non-specific binding site were blocked in PBS-BSA buffer (blocking and staining buffer: PBS pH 7.2+2% BSA) for 1 hour at room temperature. Cetuximab or mAbXcite-cetuximab were then added and the plate placed in a plate shaker for 1 hour at room temperature, followed by 3 more washes. To allow detection of the β-1,6-glucan oligomers, anti-β-1,6-glucan antibodies (polyclonal human anti-β-1,6-glucan antibodies) were added to the plate (3 ug/well) and incubated for 45 min at RT. The well were washed 3 times (PBS-Tween buffer (PH 7.2, 0.05% Tween)) and incubated with a 1:5000 dilution of anti-human IgG2 (Fc)-HRP antibody (mouse anti-human IgG2 (Fc)-HRP) for 45 min at RT. Extensive washes were performed (×5) and visualization was carried out with 100 uL of TMB substrate. Plate was read immediately at OD 620 nm (for one hour, every minute). The results shown in FIG. 12 are the mean+/−standard deviation of 3 replicate wells. Binding was evaluated by ELISA. All mAbXcite-cetuximab, conjugated with various loads, were active in this assay, and the activity increased as the load increased.

### FACS Assay

FACS studies were undertaken to assess mAbXcite-cetuximab effect on EGFR binding and anti-β-1,6-glucan IgG2 deposition. The present study utilized mAbXcite-cetuximab in which the conjugation utilized click chemistry (DBCO) using 4-mer to 9-mer oligomers at a load of 1 to 6 oligomers per antibody. Cetuximab was used as a control. Detecting antibodies included polyclonal human anti-β-1,6-glucan antibodies and mouse anti-human IgG2-PE or mouse anti-human IgG-FITC.

2×10⁵ A431 (Epithelial cells, Epidermoid carcinoma) cells were blocked for 1 hour at room temperature in PBS-BSA followed by an incubation with mAbXcite-cetuximab, cetuximab or rituximab (negative control) in staining buffer (PBS pH 7.2+2% BSA) for 1 hour at RT. Cells were then washed (×3; PBS pH 7.2), and a detection antibody (mouse anti-human IgG2-PE or mouse anti-human IgG-FITC) was added at 1:100 dilution. After three more washes, cells were spun down, re-suspended in PBS and analyzed by FACS. The cells were analyzed by FACS (FL2 for PE or FL1 for FITC) in FACSCalibur (BD Biosciences).

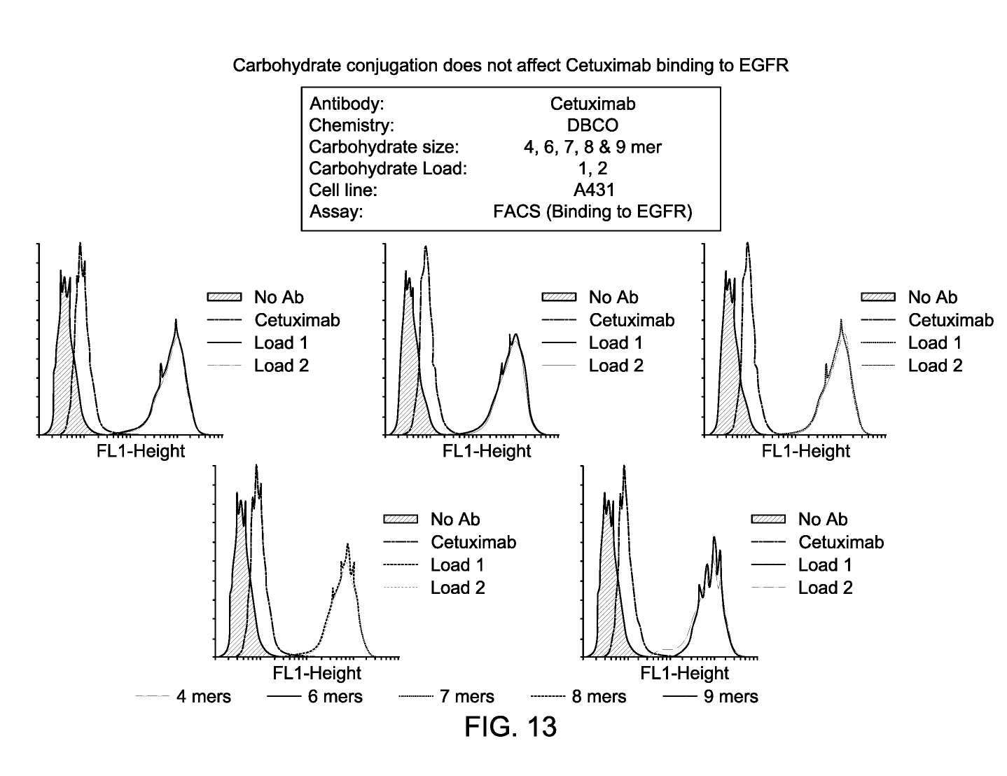

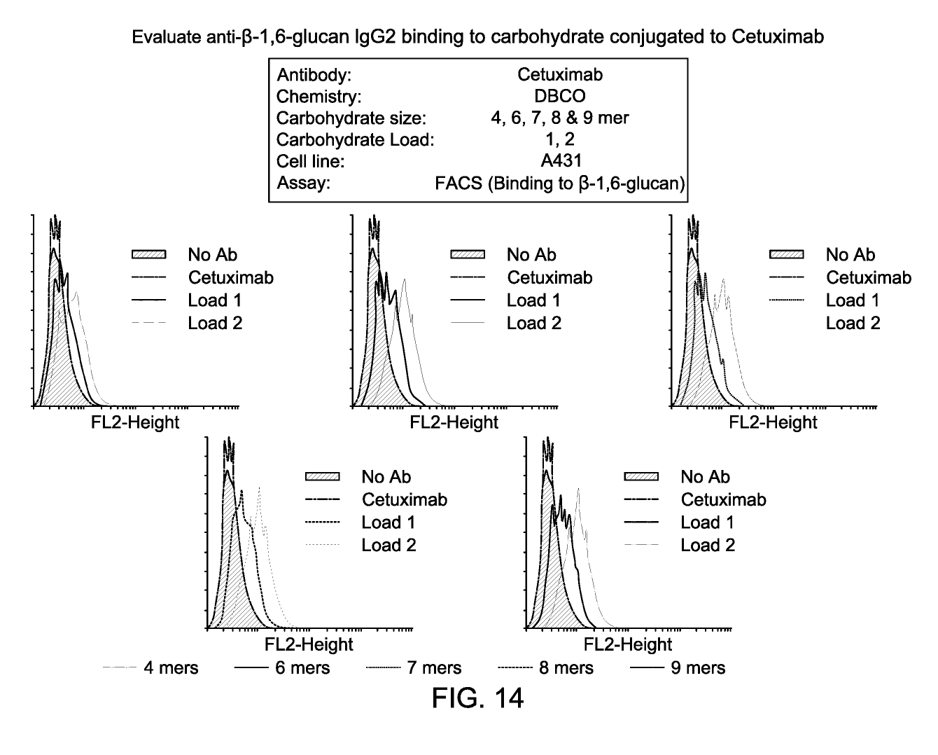

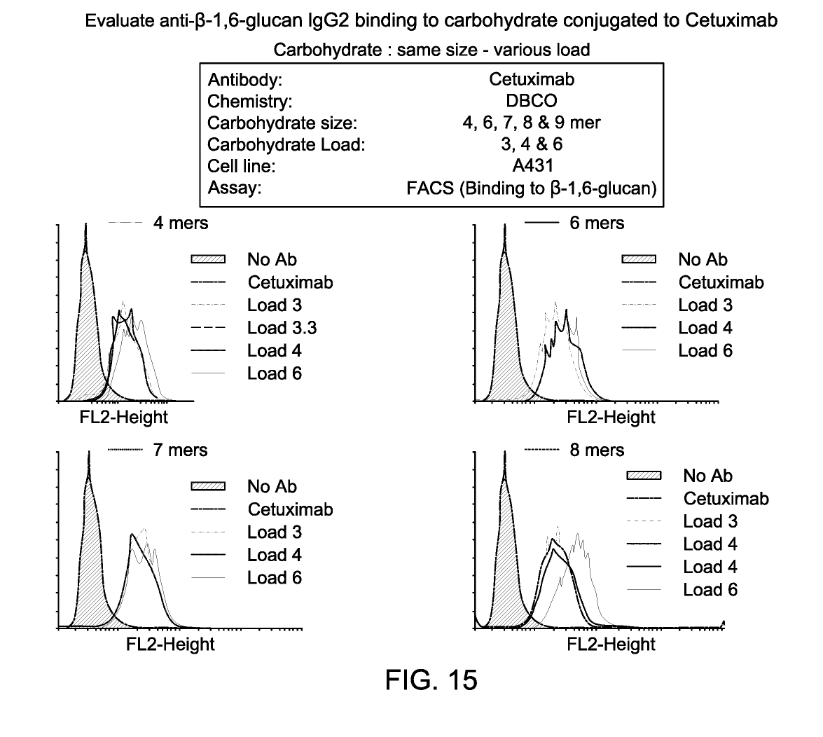

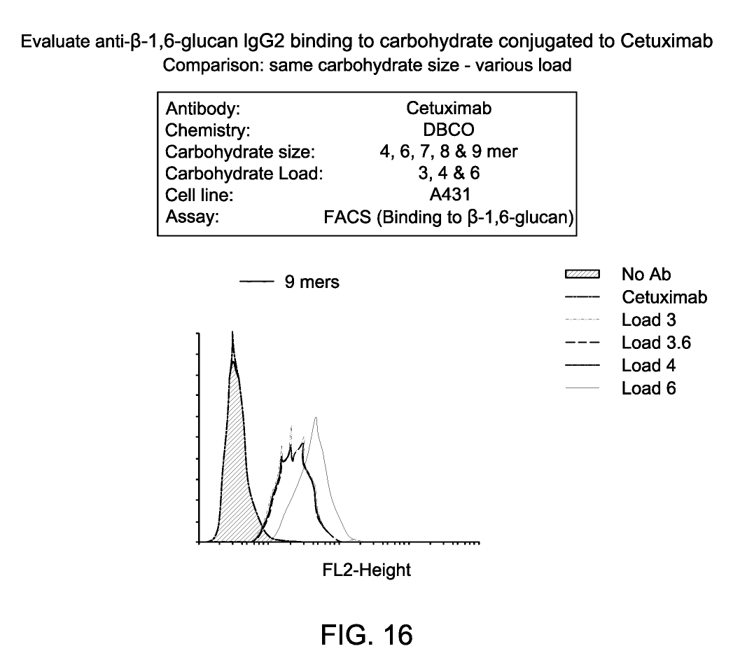

mAbXcite-cetuximab binding was assessed by FACS using buffer as control (“No Ab”), cetuximab, and mAbXcite-cetuximab (loads ranging from 1 to 6). Conjugation of cetuximab with various loads of oligomer did not affect cetuximab binding to EGFR. In addition, deposition of anti-β-1,6-glucan antibody, was not impaired with either low or high load of oligomer. A minimum load of 1, was detected by anti-β-1,6-glucan IgG2 in these assays. See FIGS. 13, 14, 15, and 16.

### Example 6: Binding of mAbXcite-Cetuximab Having Various Oligomer Loads with Anti-β-1,6-Glucan Antibody or EGFR

### ELISA Assay

The present study utilized mAbXcite-cetuximab in which the conjugation was performed by direct reductive amination chemistry, a 5-mer oligomer, and a load of 1-5 oligomers per antibody. Anti-β-1,6-glucan binding to mAbXcite-cetuximab was assayed using ELISA. Cetuximab was used as a control. ELISA was carried out using 1-Step™ Ultra TMB-ELISA (Pierce). Detecting antibodies included polyclonal human anti-β-1,6-glucan antibodies and mouse anti-human IgG2 (Fc)-HRP.

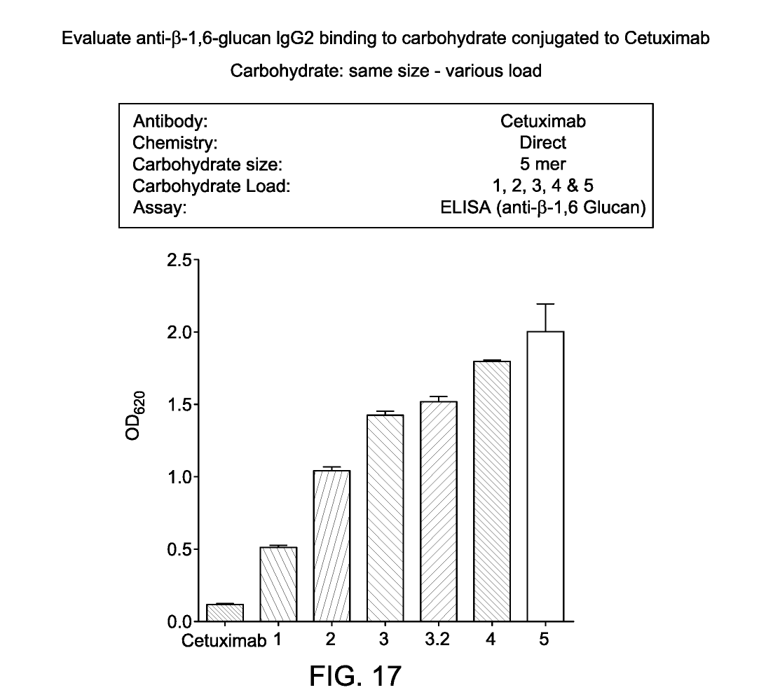

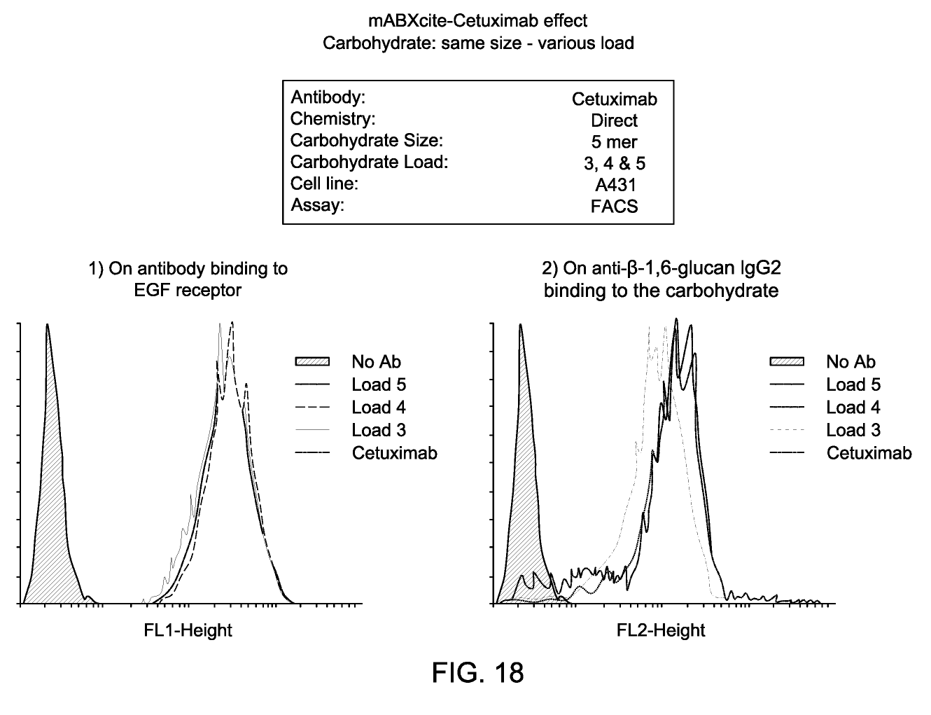

A human recombinant EGFR peptide (rhEGFR/Fc Chimera (peptide from R&D)) was absorbed on an ELISA plate (MaxiSorp) overnight at 4° C. Following a couple of washes, non-specific binding sites were blocked in PBS-BSA buffer (PBS pH 7.2+2% BSA) for 1 hour at room temperature. Cetuximab or mAbXcite-cetuximab were then added and the plate placed in a plate shaker for 1 hour at room temperature, followed by 3 more washes (pH 7.2, 0.05% Tween). To allow detection of the β-1,6-oligomers, anti-β-1,6-glucan antibodies were added to the plate (3 ug/well) and incubated for 45 min at RT. The wells were washed 3 times and incubated with a 1:5000 dilution of anti-human IgG2 (Fc)-HRP antibody for 45 min at RT. Extensive washes were performed (×5) and visualization was carried out with 100 uL of TMB substrate. Plate was read immediately at OD 620 nm (one hour, every minute). Data shown are the mean+/−standard deviation of 3 replicate wells. Similarly as what was observed with click chemistry (DBCO), all mAbXcite-cetuximab conjugates made using reductive amination were active in this assay, and activity increased as the oligomer load increased. See FIGS. 17 and 18.

### FACS Assay

These studies were undertaken to assess mAbXcite-cetuximab effect on EGFR binding and anti-β-1,6-glucan IgG2 deposition. The present study utilized mAbXcite-cetuximab in which the conjugation was performed by direct reductive amination chemistry, a 5-mer oligomer, and a load of 3-5 oligomers per antibody. Cetuximab was used as a control. Detecting antibodies included polyclonal human anti-β-1,6-glucan antibodies and mouse anti-human IgG2-PE or mouse anti-human IgG-FITC.

4×10⁵ A431 (Epithelial cells, Epidermoid carcinoma) cells were blocked for 1 hour at room temperature in PBS-BSA followed by an incubation with mAbXcite-cetuximab, cetuximab or rituximab (negative control) in staining buffer (PBS pH 7.2+2% BSA) for 1 hour at RT. Cell were then washed (×3; PBS pH 7.2), resuspended in staining buffer and half of the cells were analyzed for binding to EGFR and half for deposition of the anti-β-1,6-glucan antibody. A detection antibody (mouse anti-human IgG2-PE or mouse anti-human IgG-FITC) was added to the cells at 1:100 dilution and incubated for 45 minutes at RT. After three more washes, cells were spin down re-suspend in PBS and analyzed by FACS using FACSCalibur (BD Biosciences). mAbXcite-cetuximab binding was assessed by FACS (FL1 for FITC; FL2 for PE) using buffer control (“No Ab”), cetuximab, and mAbXcite-cetuximab (various loads) (FIG. 18). The results demonstrate that conjugation of cetuximab with the various oligomer loads does not affect cetuximab binding to EGFR. In addition, the binding of anti-β-1,6-glucan IgG2 antibodies, was not impaired with either low or high load of oligomer. The detection was similar for loads of 4 and 5, and slightly weaker for conjugates with a load of 3.

### Example 7: mAbXcite-Cetuximab Stability in Serum

In the present example, stability analysis was performed on mAbXcite-cetuximab, using cetuximab as a control. The present study utilized mAbXcite-cetuximab in which the conjugation utilized DBCO or direct chemistry, a 4-mer to 5-mer oligomer, and a load of 3 oligomers per antibody. ELISA was carried out using 1-Step™ Ultra TMB-ELISA (Pierce) detection kit. Detecting antibodies included polyclonal human anti-β-1,6-glucan antibodies, mouse anti-human IgG2 (Fc)-HRP, and goat anti-human IgG (H&L)-HRP. Also utilized were a blocking and staining buffer (casein in PBS (Pierce)) and a PBS-Tween buffer (PH 7.2, 0.05% Tween).

mAbXcite-cetuximab, or cetuximab control, were added to serum (human or mouse serum) at concentrations equivalent to a dosage of 5 mg/kg in mice (50 μg/mL) and incubated for 1, 2, 3, 4, 5, 14, 48, 72 or 96 hours at 37° C. The antibody (IgG concentration) and the presence of β-1,6-glucan oligomers were then determined by ELISA.

Sera were incubated in 96-well plates coated with recombinant EGFR (hEGFR peptide from Sino Biological). Dilution was 1:500 for detection of the antibody and 1:10 in order to detect β-1,6-glucan conjugated to cetuximab. Total human antibody binding (cetuximab or mAbXcite-cetuximab) was detected using HRP-conjugated anti-human IgG (H&L). The color was developed by TMB Substrate Kit (Pierce).

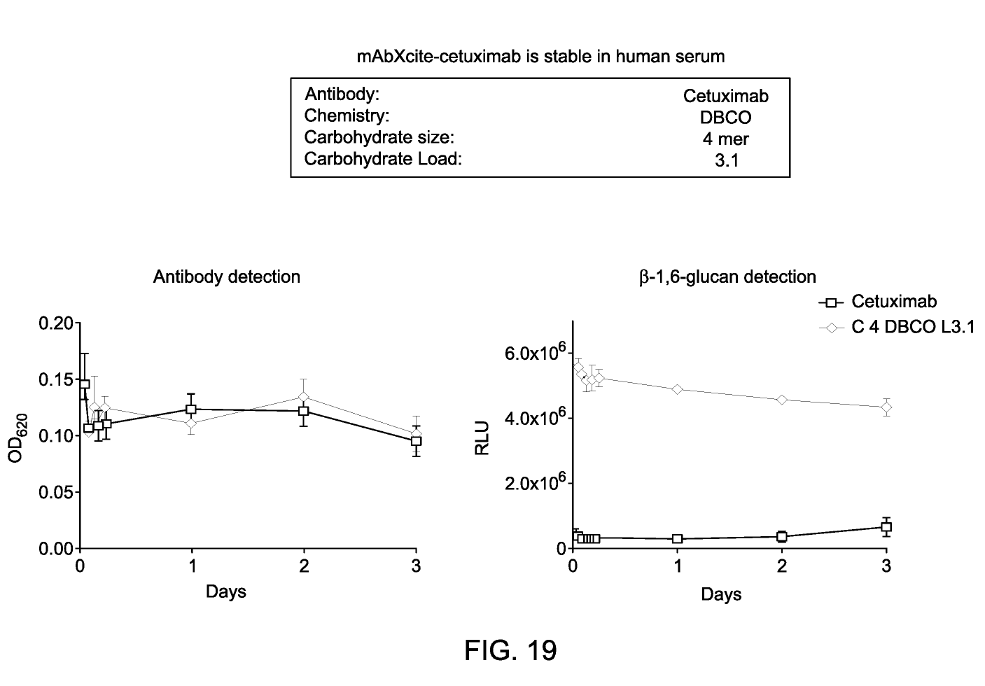

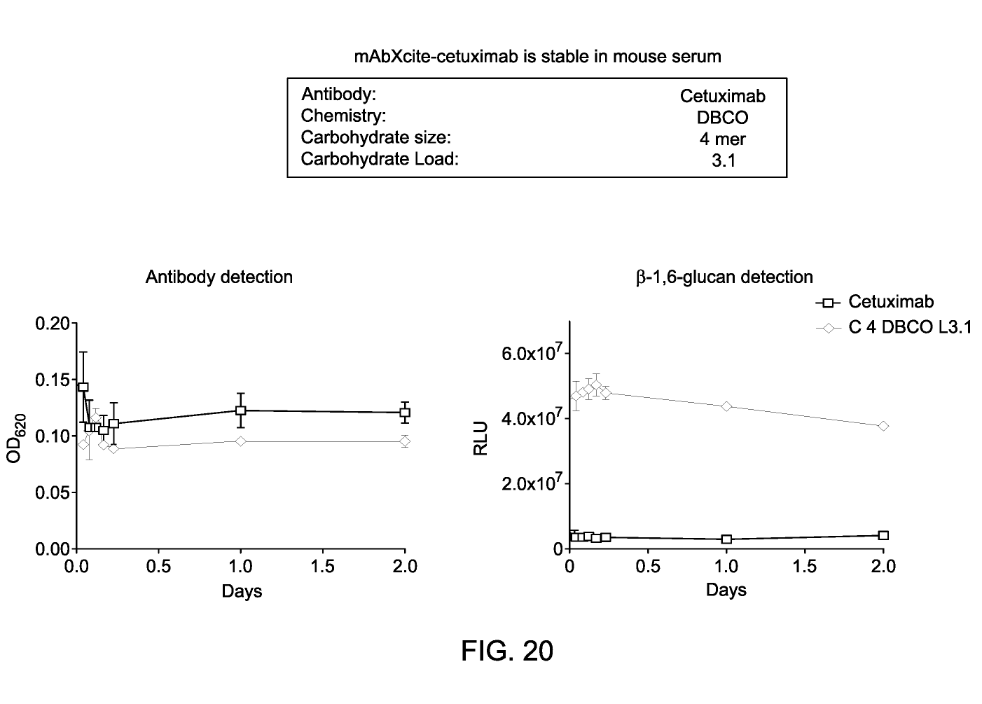

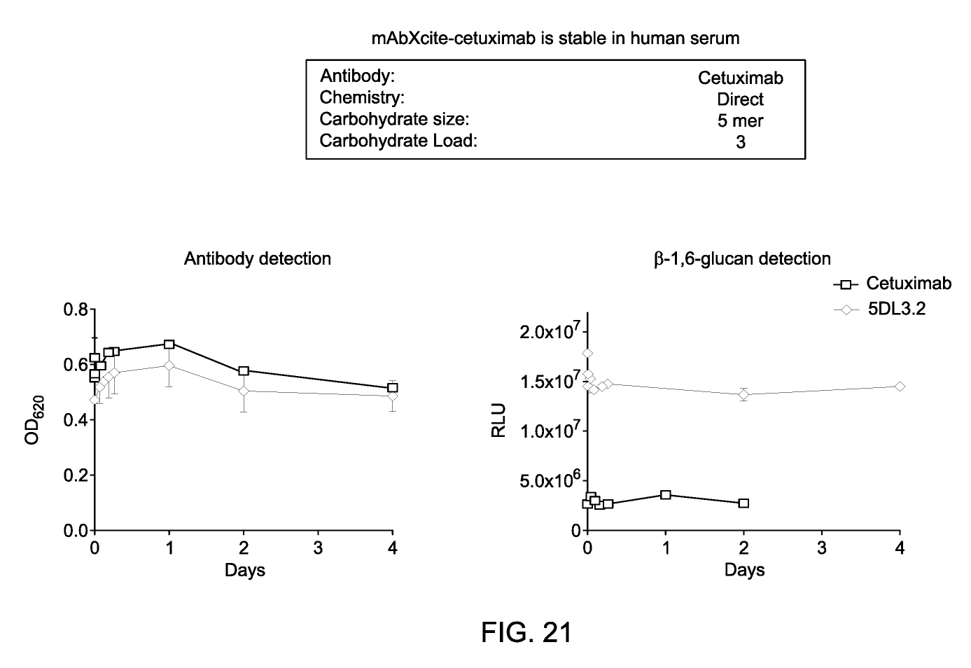

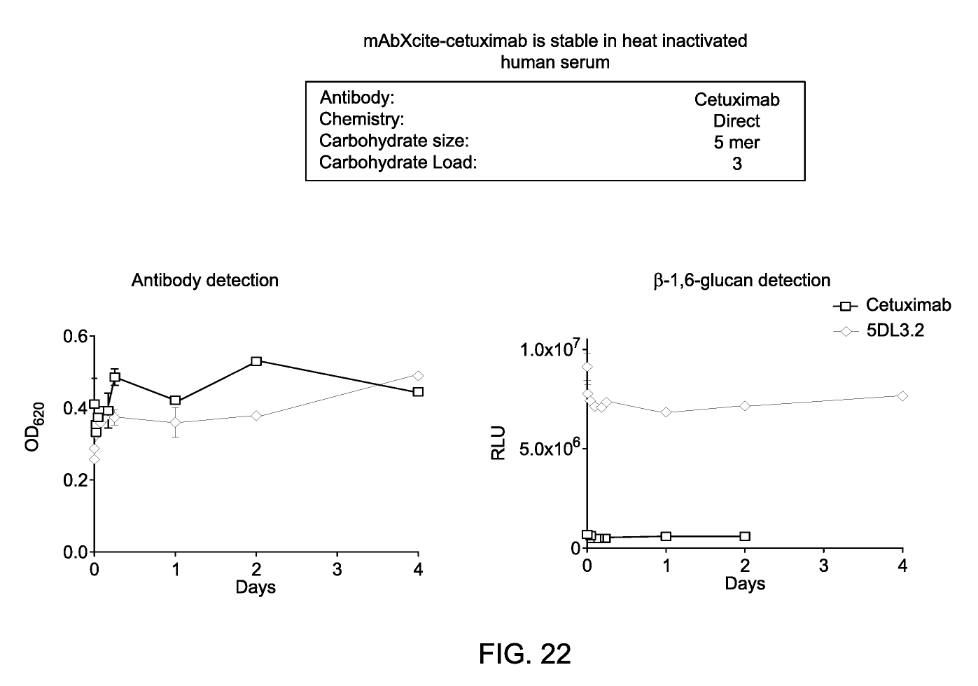

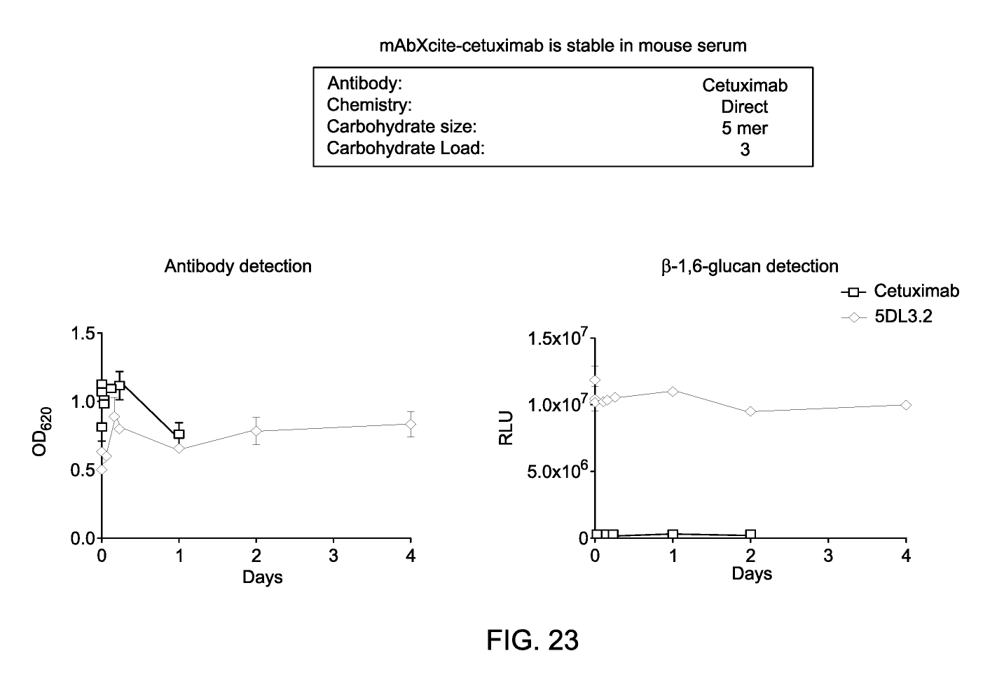

To specifically detect the β-1,6-glucan oligomers conjugated to cetuximab, affinity purified human anti-β-1,6-glucan antibodies (polyclonal human anti-β-1,6-glucan antibodies) were used (3 μg per well for 1 hour at RT). The detection was carried out with anti-human IgG2 (Fc)-HRP antibody (mouse anti-human IgG2 (Fc)-HRP). SuperSignal ELISA Substrate (SuperSignal ELISA Pico Chemiluminescent Substrate (Pierce)) was added and luminescence determined using an Envision luminometer. OD at 620 nm was read for one hour, every minute. Results from 2 replicates were averaged and standard deviation determined. The antibody level was plotted as function of time for cetuximab or mAbXcite-cetuximab. The half-life of each antibody in serum was determined as the period of time it takes to decrease by half. Cetuximab or mAbXcite-cetuximab were stable in serum at 37° C. for up to 4 days. The β-1,6-glucan oligomers were also detected, suggesting that β-1,6-glucan oligomers are stable when conjugated to the antibody. See FIGS. 19 to 23.

### Example 8: Evaluation of PK In Vitro and in Mice

The neutrophil recognition of β-1,6-glucan is opsonic, requiring endogenous IgGs, present in all human sera tested, as well as proteins of the complement system. The small size of the β-1,6-glucan oligomers that are linked to cetuximab is too short for unconjugated versions to bind these endogenous IgGs and complement proteins in circulation. They are however readily recognize by the endogenous antibodies when conjugated to cetuximab due to avidity either in plate assays or with high density of EGFR on cells. The following studies were performed to investigate the effect of oligomer length and load on PK.

### Pharmacokinetics (PK) In Vitro

This study utilized mAbXcite-cetuximab in which 5-mer to 8-mer oligomers were conjugated using direct chemistry at a load of 1, 2 and 2.5 oligomers per antibody. Cetuximab was used as a control. Detecting antibodies included polyclonal human anti-β-1,6-glucan antibodies and mouse anti-human IgG2 (Fc)-HRP. ELISA was carried out using 1-Step™ Ultra TMB-ELISA (Pierce).

A human recombinant EGFR peptide (rhEGFR/Fc Chimera (peptide from R&D)) was absorbed on an ELISA plate (MaxiSorp) overnight at 4° C. Following a couple of washes, non-specific binding sites were blocked in PBS-BSA buffer (PBS pH 7.2+2% BSA) for 1 hour at room temperature. Cetuximab or mAbXcite-cetuximab were then added and the plate placed in a plate shaker for 1 hour at room temperature, followed by 3 more washes (PBS-Tween buffer (PH 7.2, 0.05% Tween)). To allow detection of β-1,6-oligomers, anti-β-1,6-glucan antibodies were added to the plate (3 ug/well) and incubated for 45 min at RT. The wells were washed 3 times and incubated with a 1:5000 dilution of anti-human IgG2 (Fc)-HRP antibody for 45 min at RT. Extensive washes were performed (×5) and visualization was carried out with 100 uL of TMB substrate. Plate was read immediately at OD620 nm.

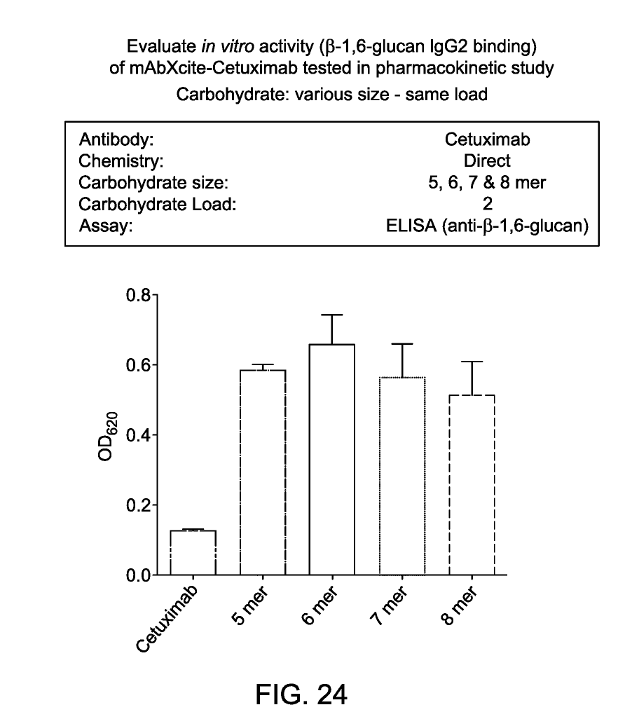

OD was read at 620 nm for one hour, every minute. Data shown are the mean+/−standard deviation of 3 replicate wells. All direct conjugates tested were active in this in vitro assay and therefore tested for their PK properties. See FIG. 24; this graph is representative of what was also observed with a load of 1 and 2.5 oligomers per antibody.

Pharmacokinetics (PK) of mAbXcite-Cetuximab in Tumor-Free Nude Mice

The present study utilized mAbXcite-cetuximab in which 5-mer to 8-mer oligomers were conjugated using direct chemistry at different loads of oligomer per antibody. Cetuximab was used as a control. The experiment further utilized a blocking and staining buffer: Casein in PBS (Pierce) and a PBS-Tween buffer (PH 7.2, 0.05% Tween). Elisa was carried out using 1-Step™ Ultra TMB-ELISA (Pierce) with SuperSignal ELISA Pico Chemiluminescent Substrate (Pierce).

PK of mAbXcite-cetuximab conjugates was determined in tumor-free nude mice in the presence of anti-β-1,6-glucan antibodies (IVIG), as they might affect the half-life of the conjugates. In these experiments, 7 week old female nude mice (Charles River) were injected IP with anti-β-1,6-glucan antibodies (polyclonal human anti-β-1,6-glucan antibodies) and, 2 hours later, with 5 mg/kg of mAbXcite-cetuximab or of cetuximab (three mice per group). To assess circulating levels of total and conjugated antibody, blood was collected by terminal bleeding at various time points post injection (from 1 hour to 96 hours). Plasma was obtained using anticoagulant-treated tubes (EDTA-treated rubes) and stored at −70° C. The antibody (IgG concentration) and the presence of the β-1,6-glucan oligomers were then determined by ELISA.

Plasma or mAbXcite-cetuximab control (as a standard) were incubated in 96-well plates coated with recombinant EGFR (hEGFR (peptide from SinoBiological)). Dilutions were 1:500 for detection of the antibody and 1:10 in order to detect β-1,6-glucan oligomers conjugated to cetuximab.

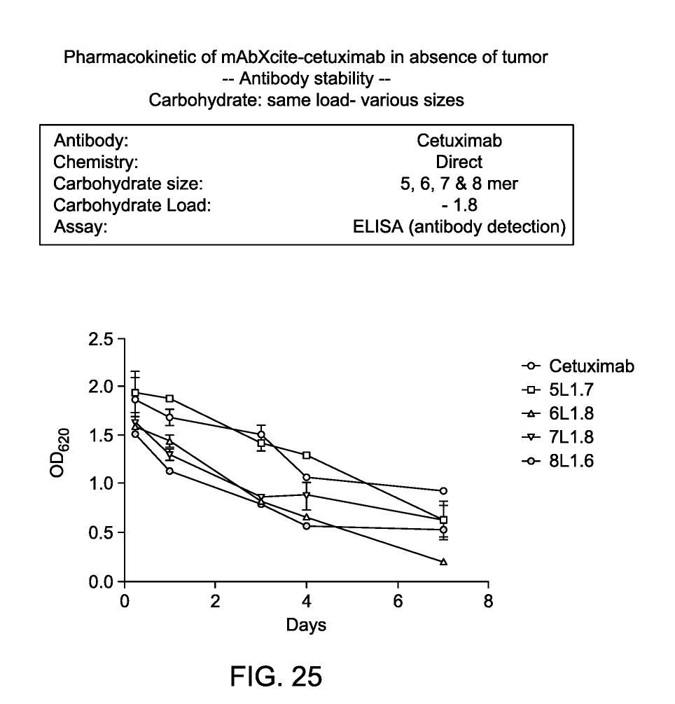

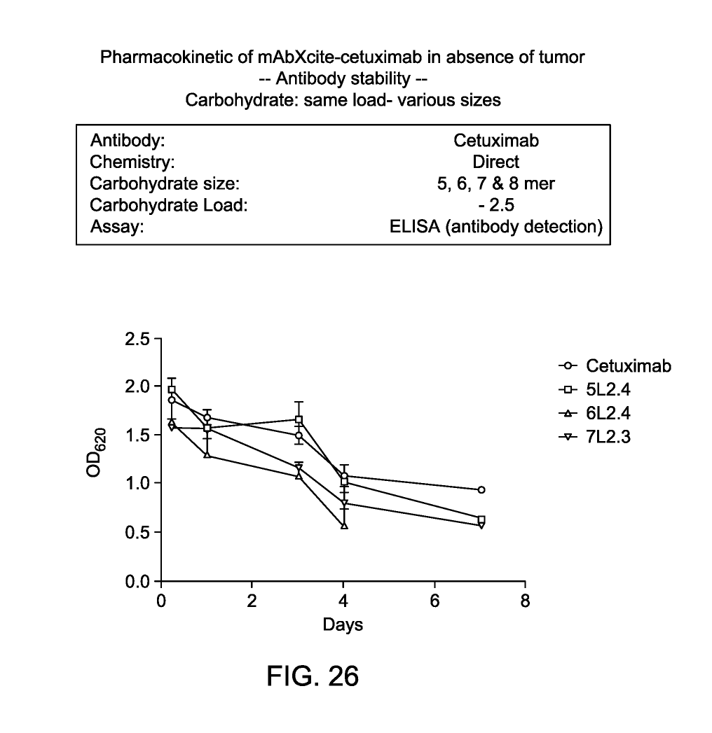

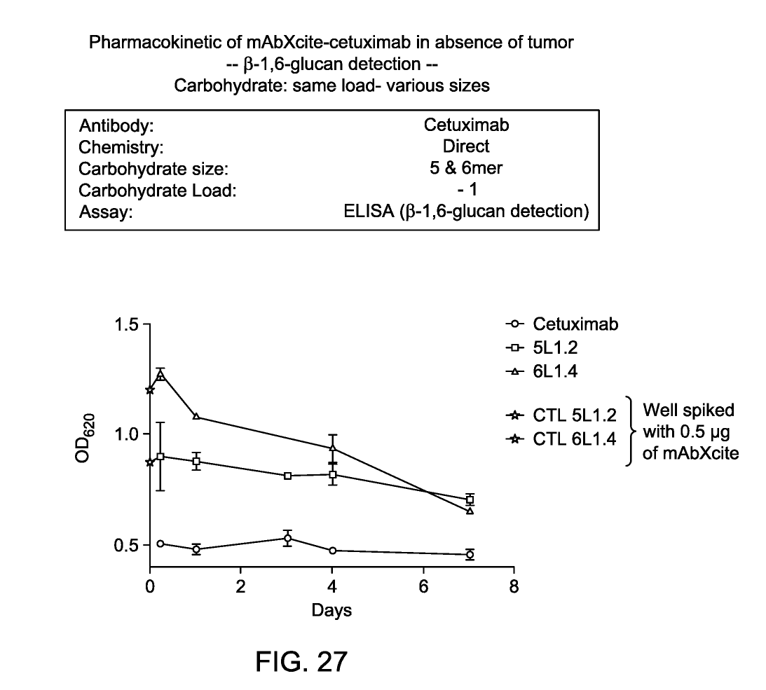

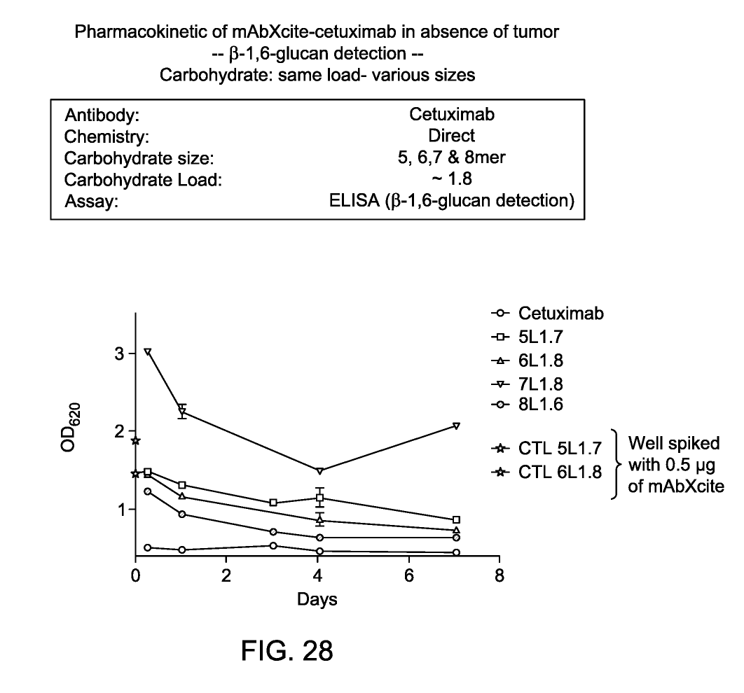

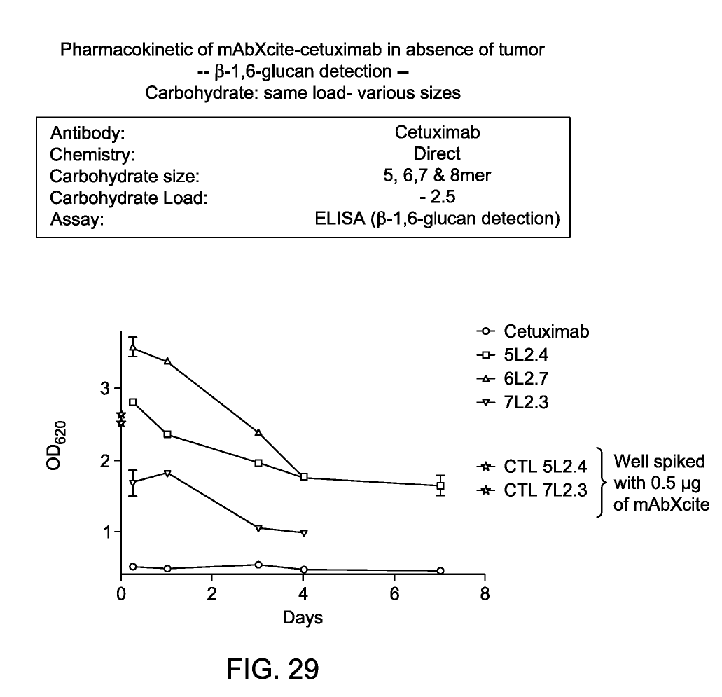

Total human antibody binding (cetuximab or mAbXcite-cetuximab) were detected using HRP-conjugated anti-human IgG (goat anti-human IgG (H&L)-HRP). To specifically detect the β-1,6-glucan oligomers linked to cetuximab, affinity purified human anti-β-1,6-glucan antibodies were used (3 μg per well for 1 hour at RT). The detection was carried out with anti-human IgG2 (Fc)-HRP antibody (mouse anti-human IgG2 (Fc)-HRP). The color was developed by TMB Substrate Kit (Pierce). OD was read at 620 nm for one hour, every minute. Results from three mice in each time point were averaged and standard error determined. See FIGS. 25 to 29. The antibody level was plotted as function of time for cetuximab or mAbXcite-cetuximab. The half-life of each antibody was determined as the period of time it takes to decrease by half. The half-life of mAbXcite-cetuximab—conjugated with a 5-mer oligomer—was similar to the half-life of cetuximab. However, mAbXcite-cetuximab clearance increased when longer oligomers were conjugated to the antibody, even at a low load level. This result suggests that mAbXcite-cetuximab conjugates with shorter oligomers may have certain advantages in vivo. Longer oligomers (5 to 8-mer) were also detected and were stable when conjugated to cetuximab.

### Example 9: Pharmacokinetics (PK) of mAbXcite-Cetuximab with Various Loads in Tumor-Free Mice

This study utilized mAbXcite-cetuximab in which 5-mer to 8-mer oligomers were conjugated using direct chemistry at different loads of oligomer per antibody. Cetuximab was used as a control. The present experiment included a blocking and staining buffer: Casein in PBS (Pierce) and a PBS-Tween buffer (PH 7.2, 0.05% Tween).

In this Example, 7 week old female nude mice (Charles River) were injected IP with anti-β-1,6-glucan antibodies (polyclonal human anti-β-1,6-glucan antibodies) and, 2 hours later, with 5 mg/kg of mAbXcite-cetuximab or cetuximab (three mice per group). To assess circulating levels of total and conjugated antibody, blood was collected by terminal bleeding at various time points post injection (from 1 hour to 96 hours). Plasma was obtained using anticoagulant-treated tubes (EDTA-treated rubes) and stored at −70° C. The antibody (IgG concentration) and the presence of the β-1,6-glucan oligomers were then determined by ELISA (1-Step™ Ultra TMB-ELISA (Pierce)).

Plasma or mAbXcite-cetuximab control (as a standard) were incubated in 96-well plates coated with recombinant EGFR (hEGFR (peptide from SinoBiological)). Dilutions were 1:500 for detection of the antibody and 1:10 in order to detect β-1,6-glucan conjugated to cetuximab.

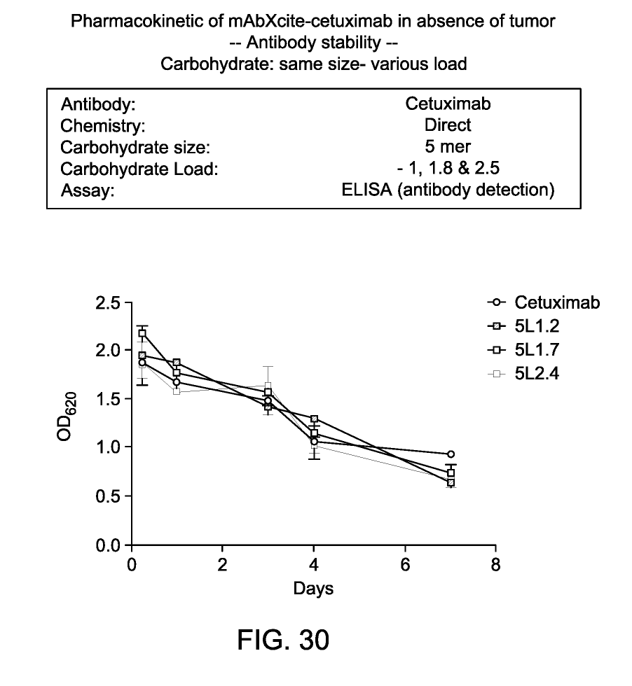

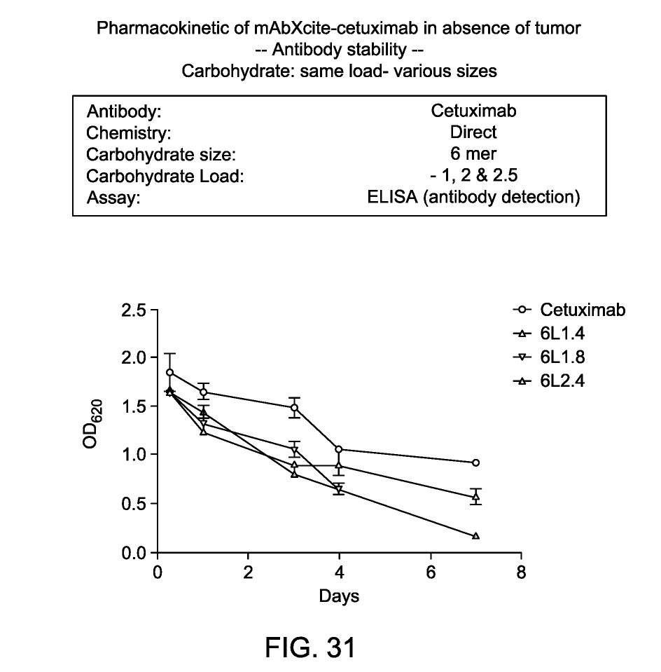

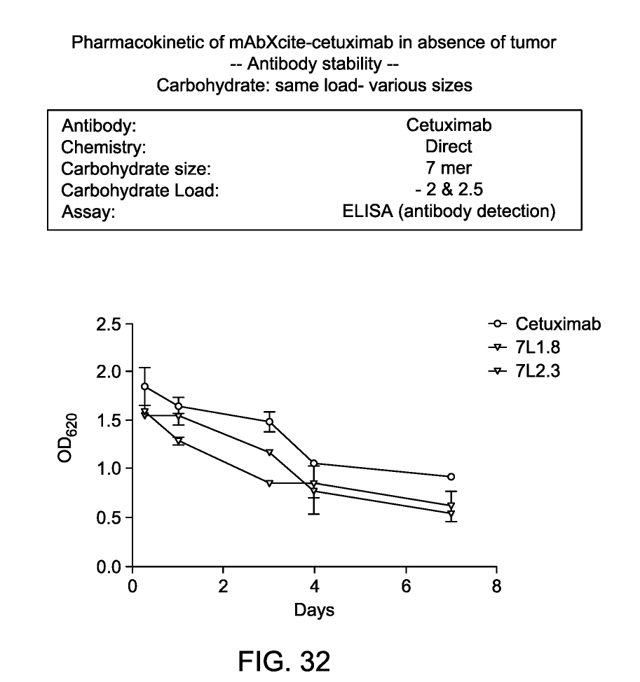

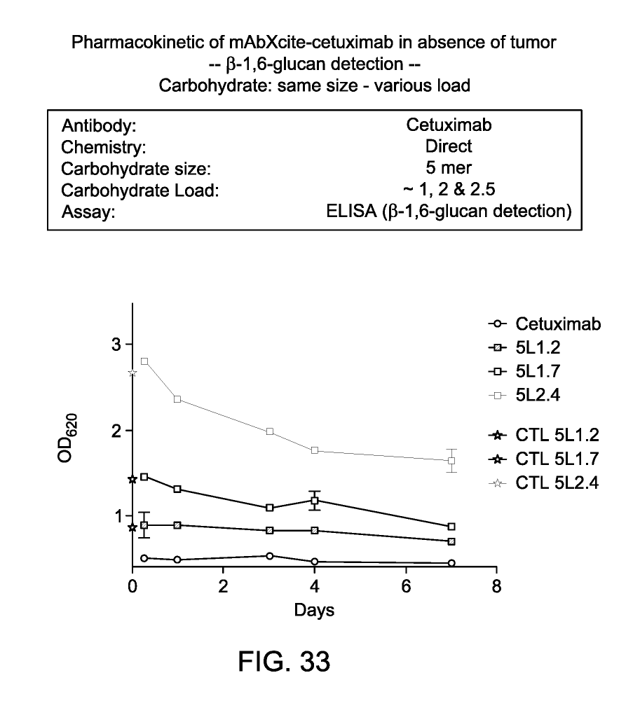

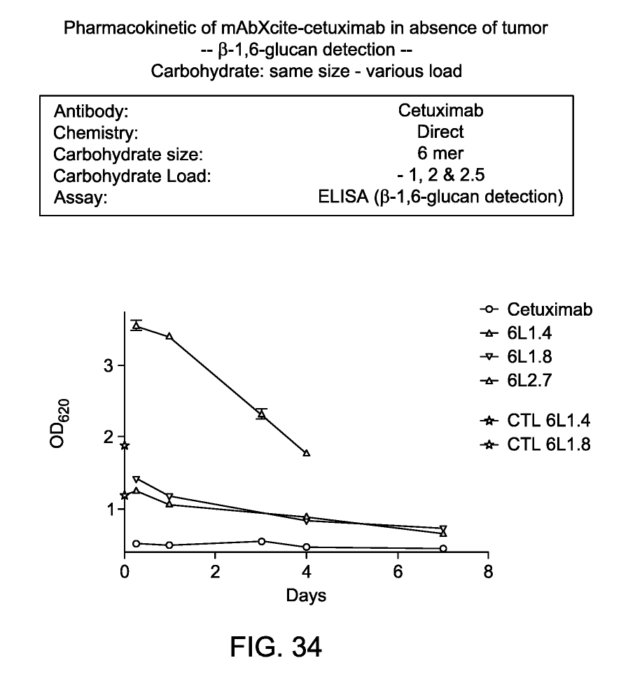

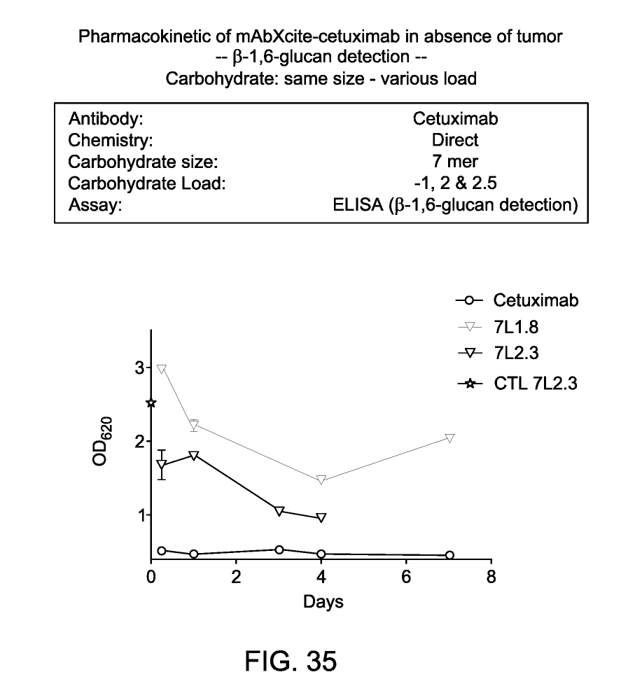

Total human antibody binding (cetuximab or mAbXcite-cetuximab) were detected using HRP-conjugated anti-human IgG (goat anti-human IgG (H&L)-HRP). To specifically detect the β-1,6-glucan oligomers linked to cetuximab, affinity purified human anti-β-1,6-glucan antibodies were used (3 μg per well for 1 hour at RT). The detection was carried out with anti-human IgG2 (Fc)-HRP antibody (mouse anti-human IgG2 (Fc)-HRP). The color was developed by TMB Substrate Kit (SuperSignal ELISA Pico Chemiluminescent Substrate (Pierce)). OD was read at 620 nm for one hour, every minute. Results from three mice in each time point were averaged and standard error determined. The antibody level was plotted as function of time for cetuximab or mAbXcite-cetuximab. The half-life of each antibody was determined as the period of time it takes to decrease by half. See FIGS. 30 to 35. The PK profile of mAbXcite-cetuximab conjugated with a 5-mer at different loads was similar to the PK profile of cetuximab. mAbXcite-cetuximab clearance was however increased when longer oligomers were conjugated to the antibody, even at low load level. β-1,6-glucan (5 to 8-mer) were stable when conjugated cetuximab. mAbXcite-cetuximab conjugates were stable with load of around 3. Oligomers were also stable with a load of around 3.

### Example 10: Pharmacokinetics (PK) of mAbXcite-Cetuximab in Tumor Bearing Mice

A large body of work has shown that the plasma clearance of many therapeutic monoclonal antibodies is dramatically enhanced in tumor bearing compared to tumor-free mice, suggesting the presence of a target mediated elimination pathway. In order to assess target mediated degradation of conjugates of the present invention, we also evaluated mAbXcite-cetuximab plasma disposition in mice bearing HCT-116 human colorectal cancer xenografts, which express EGFR.

The present study utilized mAbXcite-cetuximab in which 5-mer oligomers were conjugated using direct chemistry at a load of 3 and 4 oligomers per antibody. Cetuximab was used as a control. This study further included a blocking and staining buffer: Casein in PBS (Pierce) and a PBS-Tween buffer (PH 7.2, 0.05% Tween). ELISA was carried out using a 1-Step™ Ultra TMB-ELISA (Pierce) detection kit with SuperSignal ELISA Pico Chemiluminescent Substrate (Pierce). HCT-116 (KRAS mutant colorectal cancer) cells were used.

In this experiment, 7 week old female nude mice (Charles River) were implanted SC with 2.5×10⁶ HCT-116 cells. When tumor reached a volume superior to 120 mm³, mice were randomly divided into groups (3 mice/group) and treated with IVIG (as a source for polyclonal human anti-β-1,6-glucan antibodies) followed 2 hours later by vehicle control, cetuximab or mAbXcite-cetuximab (5 mg/kg, IP). mAbXcite-cetuximab plasma pharmacokinetics were studied at various time points (1 to 96 hours) following treatment.

Plasma or mAbXcite-cetuximab control (as a standard) were incubated in 96-well plates coated with recombinant EGFR (hEGFR (peptide from SinoBiological)). Dilutions were 1:500 for detection of the antibody and 1:10 in order to detect β-1,6-glucan oligomers conjugated to cetuximab. Total human antibody binding (cetuximab or mAbXcite-cetuximab) were detected using HRP-conjugated anti-human IgG (Goat anti-human IgG (H&L)-HRP). To specifically detect β-1,6-glucan oligomers linked to cetuximab, affinity purified human anti-β-1,6-glucan antibodies were used (3 μg per well for 1 hour at RT). The detection was carried out with anti-human IgG2 (Fc)-HRP antibody (mouse anti-human IgG2 (Fc)-HRP). The color was developed by TMB Substrate Kit (Pierce).

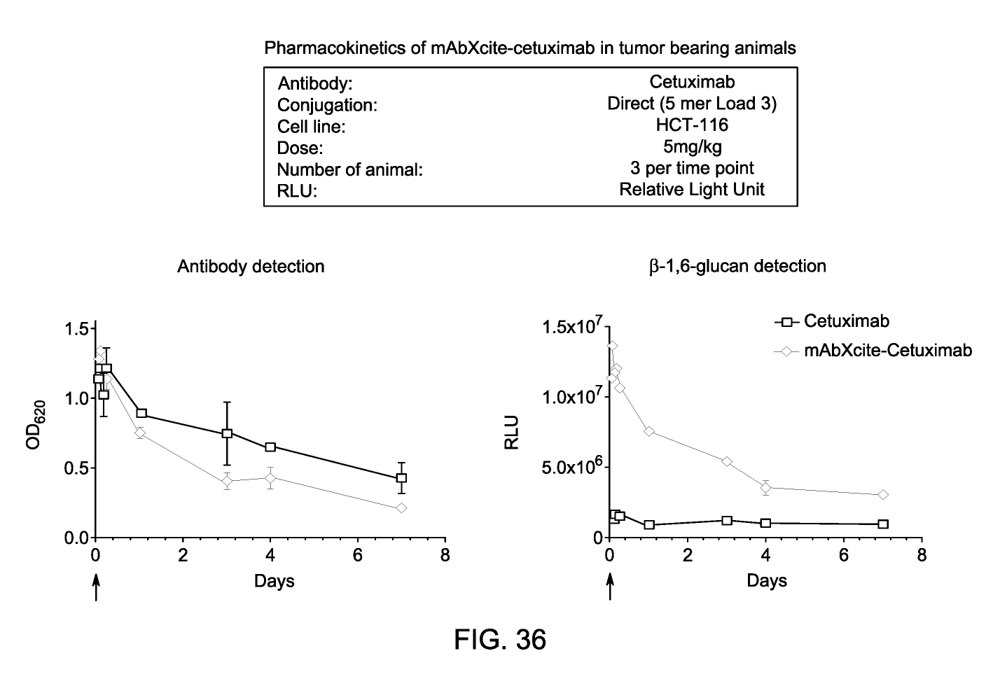

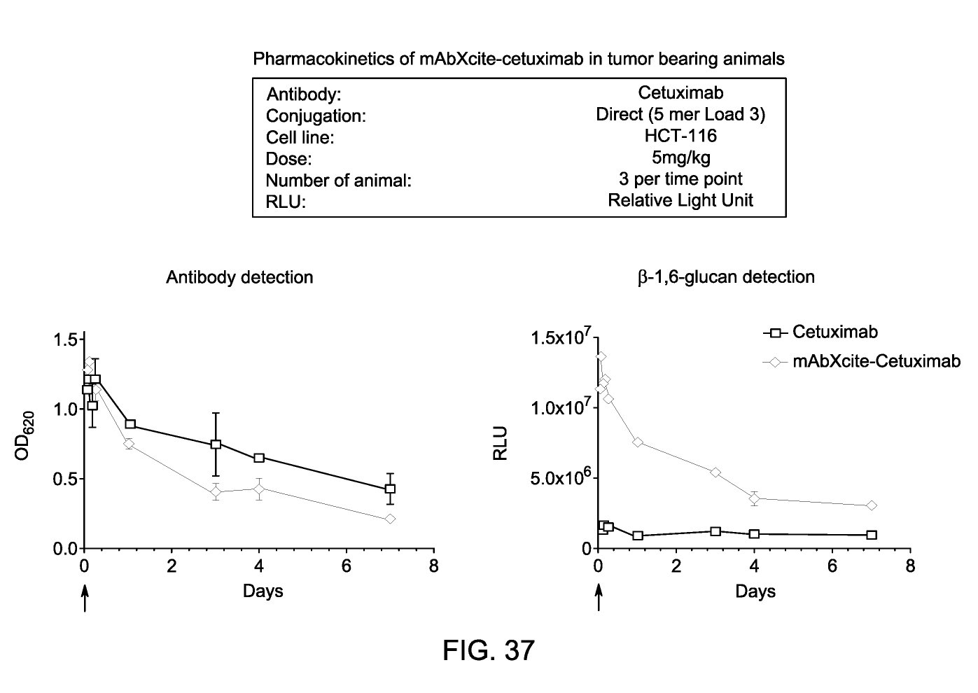

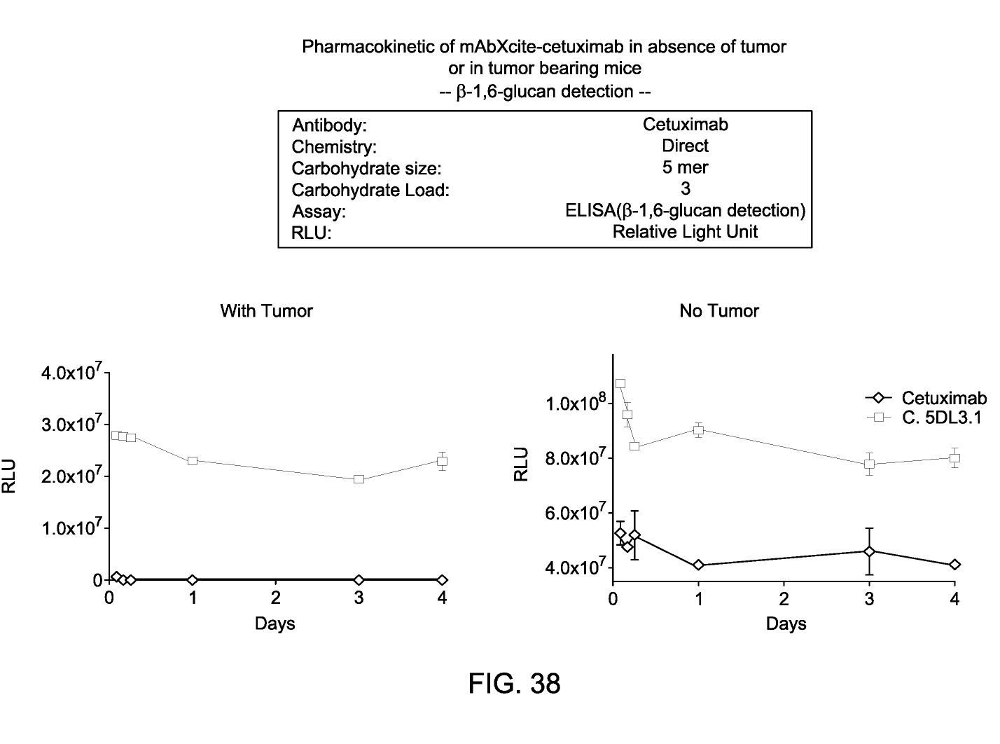

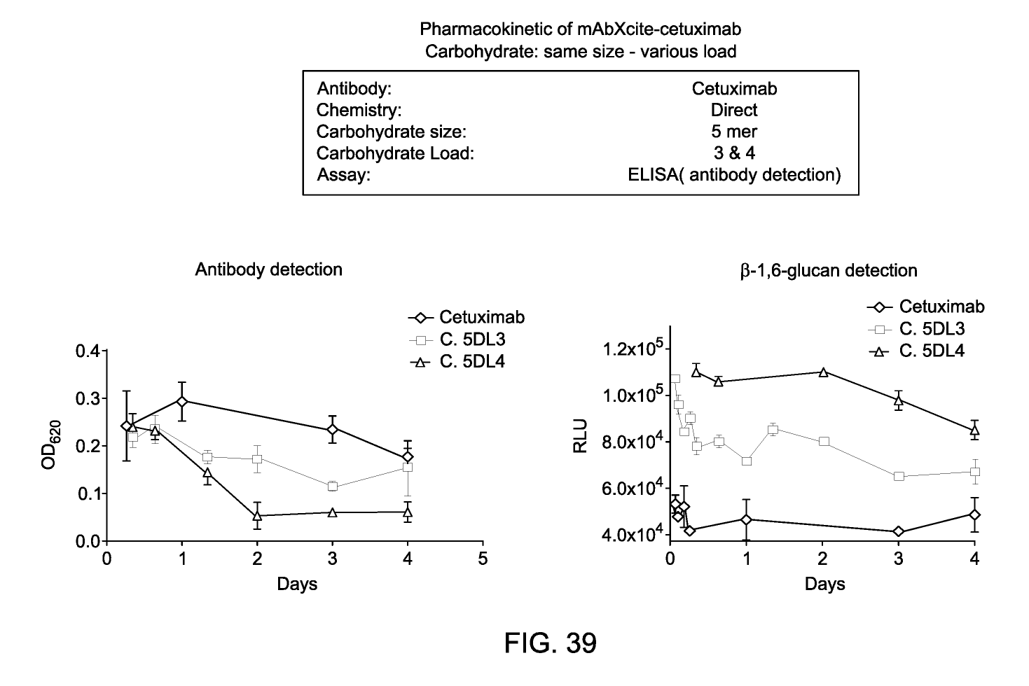

Analysis of data collected form this experiment was performed. Tumor Volume was calculated based on the following formula TV (mm3)={length (mm)×width (mm)2}/2. For ELISA analysis, OD was read at 620 nm for one hour, every minute. Results from three mice at each time point were averaged and standard error determined. See FIGS. 36 to 38. The antibody level was plotted as function of time for cetuximab or mAbXcite-cetuximab. The half-life of each antibody was determined as the period of time it takes to decrease by half. FIG. 38 provides a comparison of mAbXcite-cetuximab PK in tumor-free and tumor bearing mice. Stability of the antibody and β-1,6-glucan detection were assessed by ELISA. FIG. 39 provides a comparison of mAbXcite-cetuximab conjugated with 3 or 4 β-1,6-oligomers in tumor bearing mice. Stability of the antibody and β-1,6-glucan detection were assessed by ELISA. PK of mAbXcite-cetuximab directly conjugated to a 5-mer oligomer was more stable with a load of 3 than with a load of 4 oligomers per antibody. Tumor-dependent degradation may explain lower PK in tumor bearing animals with higher load of oligomers.

### Example 11: Accumulation of mAbXcite-Cetuximab in Tumor Bearing Mice

The studies of the present Example were undertaken to identify dosing regimens that do not result in antibody accumulation over time.

The present Example utilized mAbXcite-cetuximab in which 5-mer oligomers were conjugated using direct chemistry at a load of 3 oligomers per antibody. Cetuximab was used as a control. Also utilized were a blocking and staining buffer (casein in PBS (Pierce)) and a PBS-Tween buffer (pH 7.2, 0.05% Tween). ELISA was carried out using a 1-Step™ Ultra TMB-ELISA (Pierce) detection kit.

In this Example, 7 week old female nude mice (Charles River) were implanted SC with 2.5×10⁶ HCT-116 (KRAS mutant colorectal cancer) cells. When tumor reached a volume of 120 mm³, mice were randomly divided into groups (3 mice/group) and treated with IVIG (as a source for polyclonal human anti-β-1,6-glucan antibodies) followed 2 hours later by vehicle control, cetuximab, or mAbXcite-cetuximab. The dosing was determined by the half-life and was twice weekly (5 mg/kg). Mice were dosed 3 times and mAbXcite-cetuximab plasma collected to study conjugate accumulation following treatment.

Plasma or mAbXcite-cetuximab control (as a standard) were incubated in 96-well plates coated with recombinant EGFR (hEGFR (peptide from SinoBiological)). Dilutions were 1:500 for detection of the antibody and 1:10 in order to detect β-1,6-glucan oligomers conjugated to cetuximab.

Total human antibody binding (cetuximab or mAbXcite-cetuximab) was detected using HRP-conjugated anti-human IgG (goat anti-human IgG (H&L)-HRP). To specifically detect the β-1,6-glucan oligomers linked to cetuximab, affinity purified human anti-β-1,6-glucan antibodies were used (3 μg per well for 1 hour at RT). The detection was carried out with anti-human IgG2 (Fc)-HRP antibody (mouse anti-human IgG2 (Fc)-HRP). The color was developed by TMB Substrate Kit (SuperSignal ELISA Pico Chemiluminescent Substrate (Pierce)). For ELISA analysis, OD was read at 620 nm for one hour, every minute.

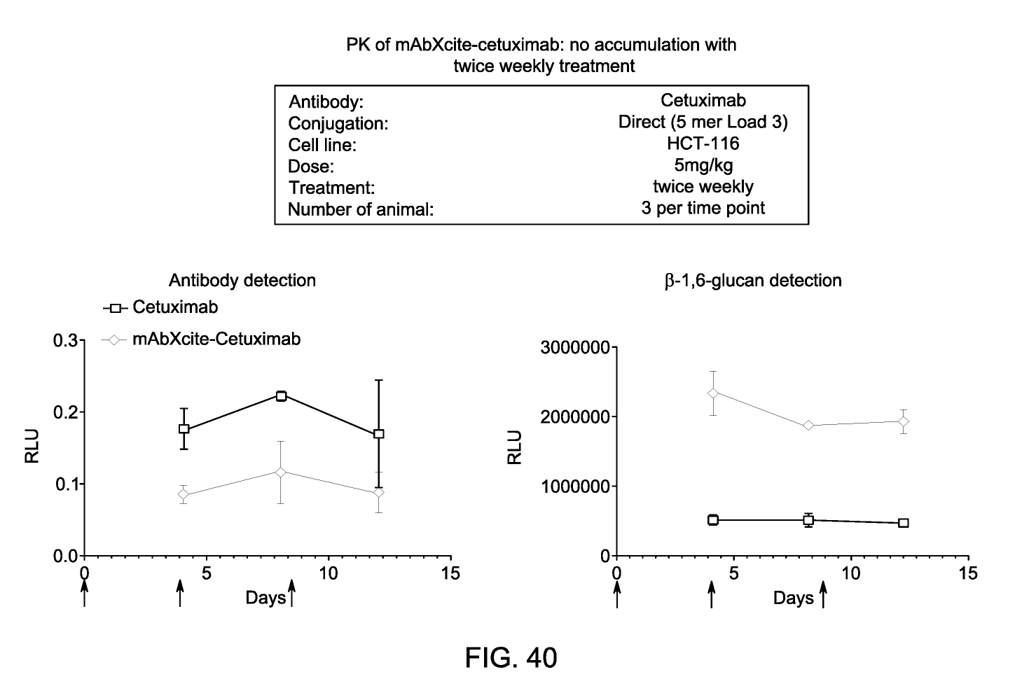

Analysis of data collected from this experiment was performed. Tumor volume was calculated based on the following formula: TV (mm³)={length (mm)×width (mm)²}/2. Results from three mice at each time point were averaged and standard error determined. See FIG. 40. The antibody level was plotted as function of time for cetuximab or mAbXcite-cetuximab. The half-life of each antibody was determined as the period of time it takes to decrease by half. A twice weekly dosing regimen, based on the half-life determined by PK, does not lead to either antibody or oligomer accumulation. This treatment schedule was used in subsequent examples to assess mAbXite-cetuximab efficacy in a mouse xenograft model.

### Example 12: Pharmacokinetic Stability of mAbXcite-Cetuximab in Tumor-Free and Tumor-Bearing Mice

Stability of the antibody and β-1,6-glucan detection were assessed by ELISA. The present study utilized mAbXcite-cetuximab in which the conjugation utilized click chemistry (DBCO), a 4-mer oligomer and a load of 3 oligomers per antibody. Cetuximab was used as a control. Also utilized were a blocking and staining buffer: Casein in PBS (Pierce) and a PBS-Tween buffer (PH 7.2, 0.05% Tween). ELISA was carried out using a 1-Step™ Ultra TMB-ELISA (Pierce) detection kit.

In this Example, 7 week old female nude mice (Charles River) were implanted SC with 2.5×10⁶HCT-116 (KRAS mutant colorectal cancer) cells. When tumor reached a volume superior to 120 mm³, mice were randomly divided into groups (3 mice/group) and treated with IVIG (as a source for polyclonal human anti-β-1,6-glucan antibodies) followed 2 hours later by vehicle control, cetuximab or mAbXcite-cetuximab (5 mg/kg, IP). mAbXcite-cetuximab plasma pharmacokinetics were studied at various time points (1 to 96 hours) following treatment.

Plasma or mAbXcite-cetuximab control (as a standard) were incubated in 96-well plates coated with recombinant EGFR (hEGFR (peptide from SinoBiological)). Dilutions were 1:500 for detection of the antibody and 1:10 in order to detect β-1,6-glucan oligomers conjugated to cetuximab.

Total human antibody binding (cetuximab or mAbXcite-cetuximab) were detected using HRP-conjugated anti-human IgG (goat anti-human IgG (H&L)-HRP). To specifically detect the β-1,6-glucan oligomers linked to cetuximab, affinity purified human anti-β-1,6-glucan antibodies were used (3 μg per well for 1 hour at RT). The detection was carried out with anti-human IgG2 (Fc)-HRP antibody (mouse anti-human IgG2 (Fc)-HRP). The color was developed using SuperSignal ELISA Pico Chemiluminescent Substrate (Pierce). For ELISA analysis, OD was read at 620 nm for one hour, every minute.

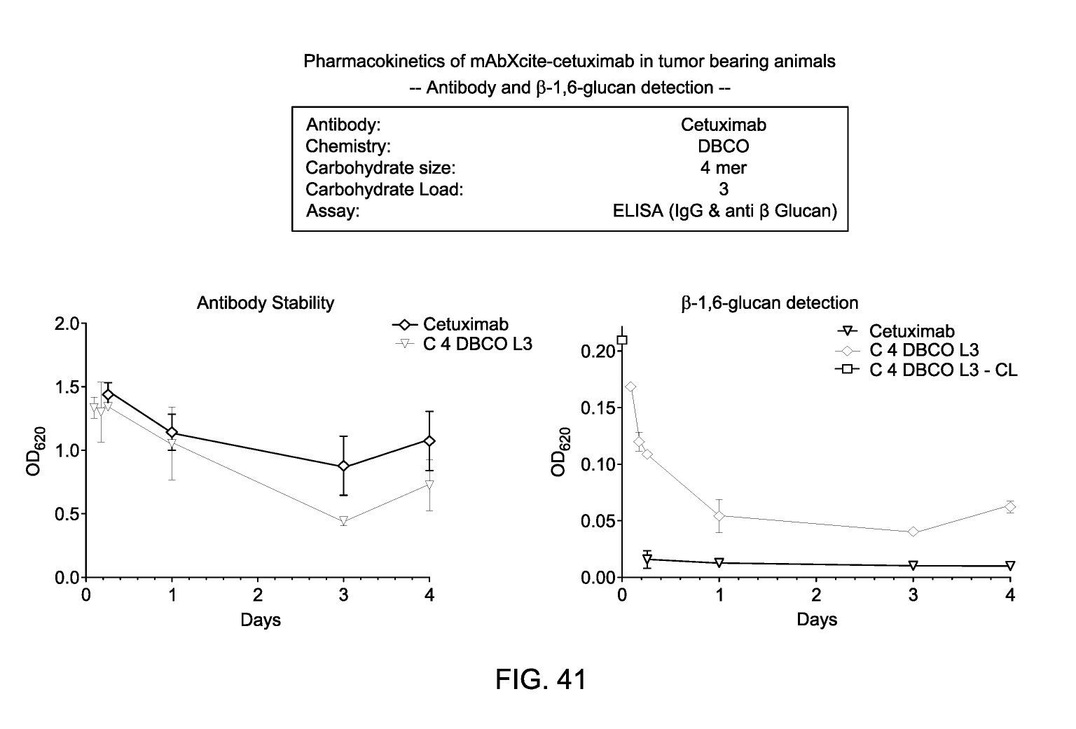

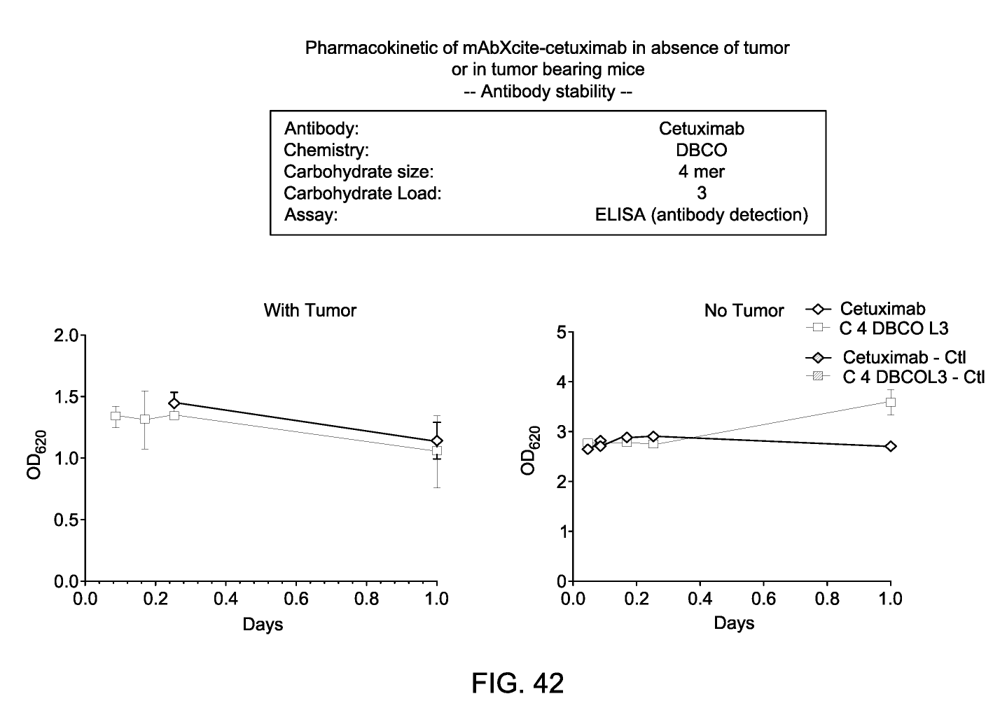

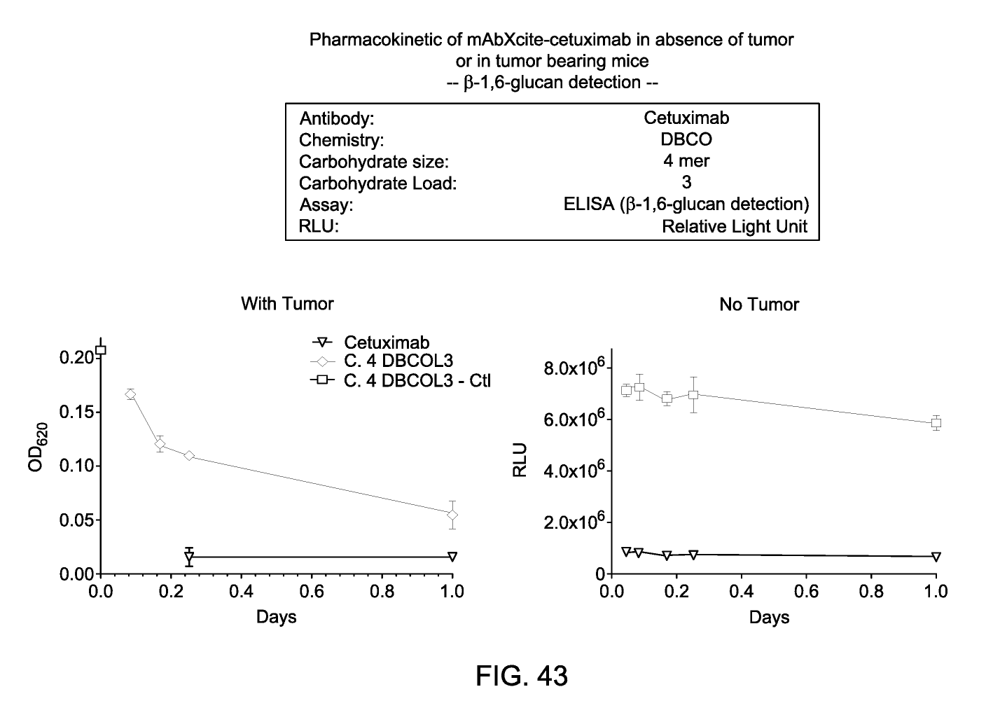

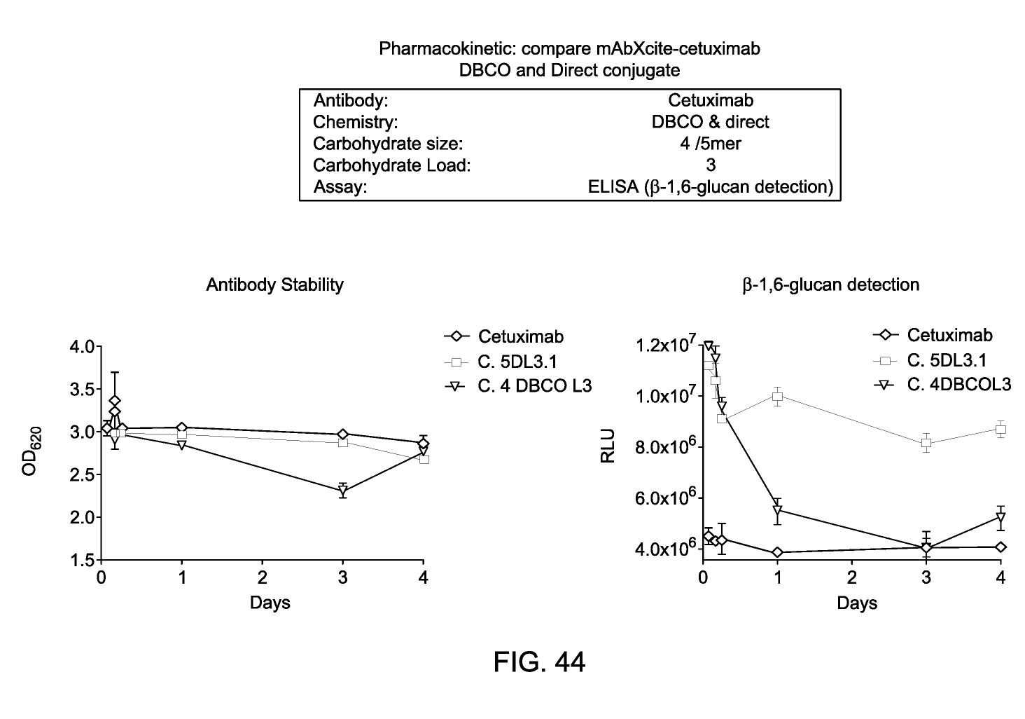

Analysis of data collected from this experiment was performed. Tumor Volume was calculated based on the following formula: TV (mm³)={length (mm)×width (mm)²}/2. Results from three mice at each time point were averaged and standard error determined. See FIGS. 41 to 44. The antibody level was plotted as function of time for cetuximab or mAbXcite-cetuximab. The half-life of each antibody was determined as the period of time it takes to decrease by half. Contrary to what was observed with the directly conjugated mAbXcite-cetuximab, PK profile of this mAbXcite-cetuximab was dramatically decreased in tumor bearing mice. Antibody remained relatively stable but β-1,6-glucan oligomer was not stable even with a small oligomer (4-mer) and a load of 3.

### Example 13: Efficacy Study in Nude Mice with BRAF Colorectal Cancer Cell (CRC)

The present study utilized mAbXcite-cetuximab in which the conjugation of the glucan utilized click chemistry (DBCO), 4-mer and 6-mer oligomers, and loads of 3 and 4 oligomer per antibody.

In this Example, 7 week old female nude mice (Charles River) were implanted with 1×10⁶HT-29 cells (a human BRAF mutant colorectal adenocarcinoma cell line) into the dorsal region. Once tumors reached approximately 118-383 mm³, mice were randomly assigned into four study treatment groups (n=5), such that the mean tumor volumes for each group were similar (mean=255.8±0.8 mm³). Prior to each antibody administration, all mice received a 500 mg/kg (˜0.1 mL) intraperitoneal (IP) injection of pooled human antibodies (IVIG). mAbXcite-cetuximab, cetuximab (5 mg/kg) and control were administered via an IP injection a minimum of 2 hours post-IVIG administration. Throughout the study, tumors were monitored twice weekly to determine tumor growth rates using external calipers. Tumors were measured prior to and following the first day of treatment. Body weights were also collected twice a week. Mice were euthanized when the maximum tumor volume reached 2000 mm³. Mice were also to be euthanized if the tumor became ulcerated, if the tumor impeded ambulation, or there was a deterioration of body condition. Mice were also to be euthanized if they lost >15% of their original body weight. Animals were euthanized via carbon dioxide (CO₂) inhalation.

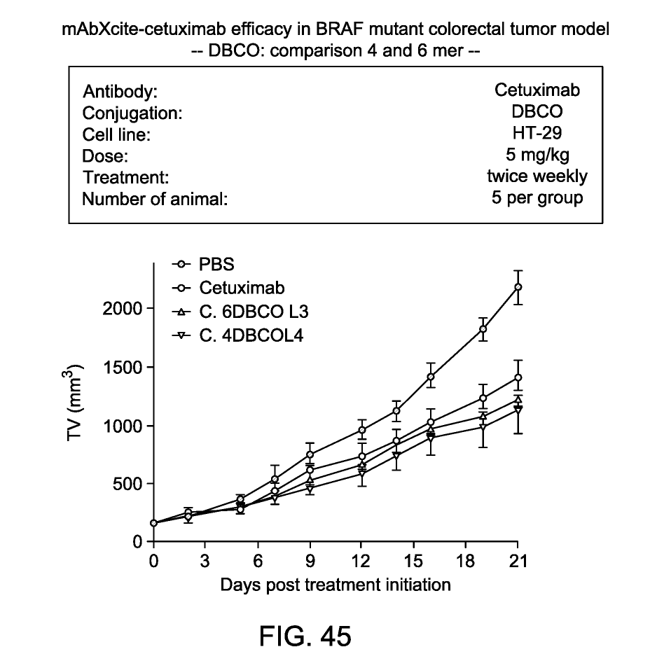

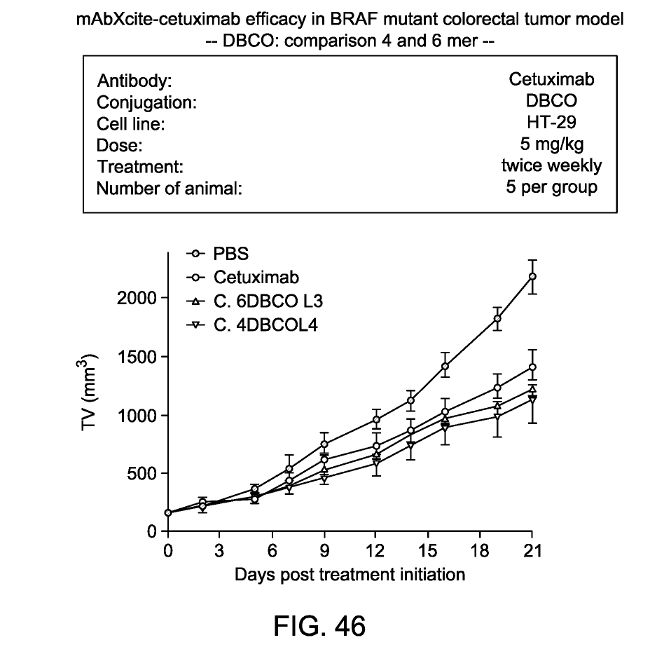

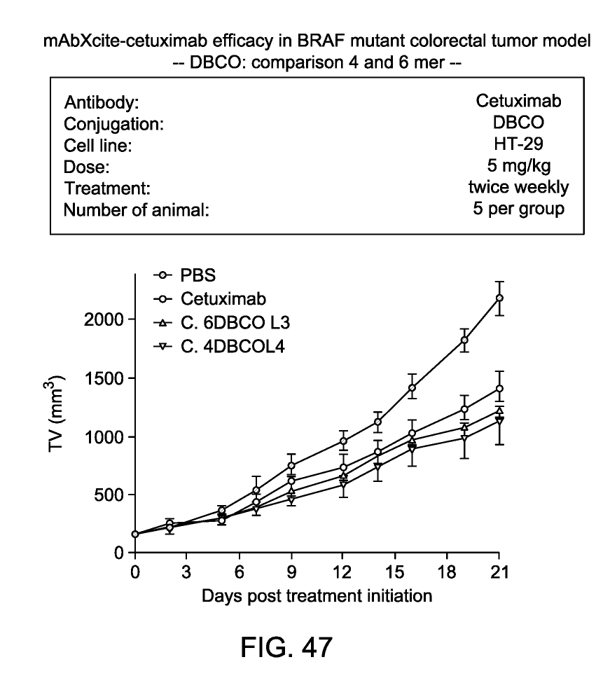

Analysis of data collected form this experiment was performed. Tumor volume was calculated based on the following formula: TV (mm³)={length (mm)×width (mm)²}/2. Results are shown in FIGS. 45 to 47. mAbXcite-cetuximab treatment with a 4-mer DBCO linked oligomer delayed the growth of HT-29 tumor. Borderline activity was seen with a 6-mer DBCO linked oligomer. It is to be noted that in that experimental setting, cetuximab also slows tumor growth.

### Example 14: Efficacy Study in Nude Mice with KRAS Colorectal Cancer Cell (CRC)

In this Example, the efficacy of mAbXcite-cetuximab was studied in nude mice that were treated with KRAS CRC. The present study utilized mAbXcite-cetuximab in which the conjugation of the glucan utilized click chemistry (DBCO) or direct chemistry, 4-mer to 6-mer oligomers, and a load of 3 oligomers per antibody.

In this Example, 7 week old female nude mice (Charles River) were implanted SC with 2.5×10⁶HCT-116 cells (HCT-116 is a human KRAS mutant colorectal cancer cell line). When tumor volume was in the range of 67±1.3 mm³ mice were randomly divided into groups (10 mice/group) and treated with IVIG (as a source for anti-β-1,6-glucan antibodies) followed 2 hours later by vehicle control, cetuximab or mAbXcite-cetuximab. The dosing was determined by the half-life and was twice weekly (5 mg/kg). Tumor volumes were assessed by caliper measurement twice weekly. Body weights were monitored twice weekly. Mice were euthanized after tumors reached a volume of 2000 mm³ or if their body weight dropped by more than 15%.

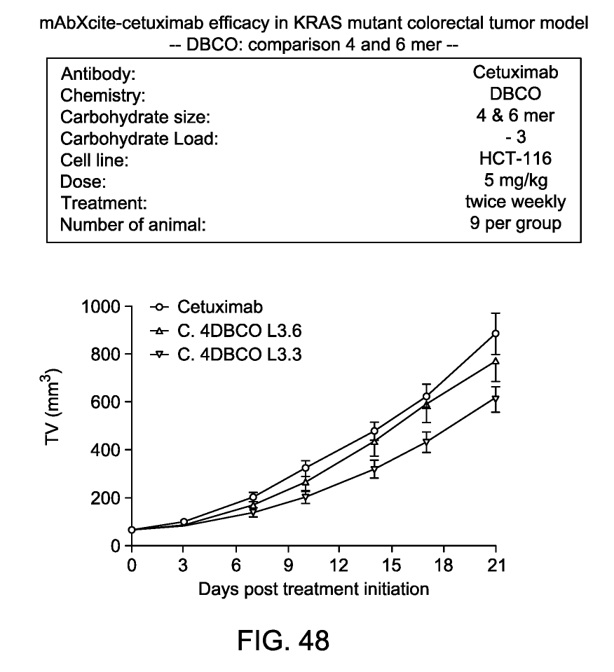

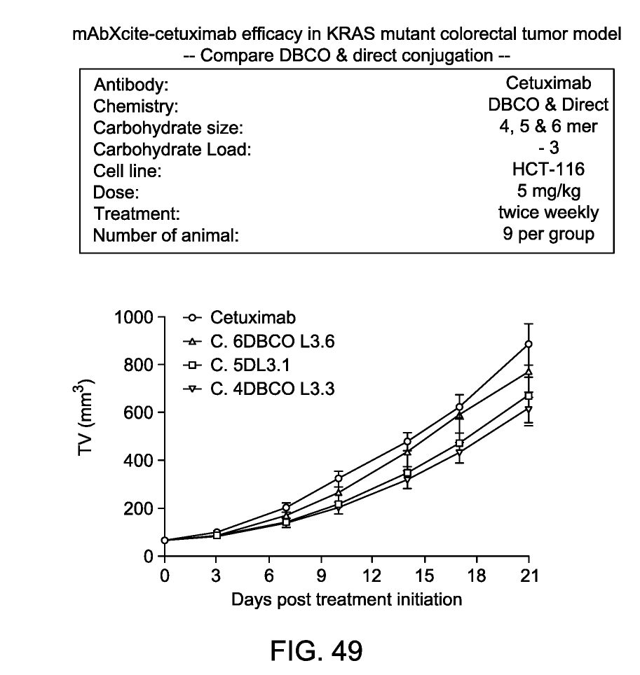

Analysis of data collected form this experiment was performed. Tumor volume was calculated based on the following formula: TV (mm³)={length (mm)×width (mm)²}/2. Results are shown in FIGS. 48 and 49. mAbXcite-cetuximab treatment with a 4-mer DBCO linked oligomers delays the growth of HCT-116 tumor. However, 6-mer DBCO linked oligomers had no effect on tumor growth in that experiment. These results confirm the results obtained in the HT-29 xenograft model and suggest that shorter oligomers may have certain advantages which may be explained by the better PK profile of these mAbXcite-cetuximab conjugates. The growth inhibition was similar in this experiment with mAbXcite-cetuximab with a 4-mer DBCO linked oligomer and 5-mer direct conjugates.

### Example 15: Efficacy Study in Nude Mice with KRAS CRC

In this Example, the efficacy of mAbXcite-cetuximab was studied in nude mice with KRAS CRC. The present study utilized mAbXcite-cetuximab in which the conjugation utilized reductive amination (direct) chemistry, a 5-mer oligomer, and a load of 3 oligomers per antibody.

In this Example, 7 week old female nude mice (Charles River) implanted with 2.5×10⁶HCT-116 cells (HCT-116 is a human KRAS mutant colorectal cancer cell line). Once tumors reached approximately 46-79 mm³, mice were randomly assigned into four study treatment groups (n=9), such that the mean tumor volumes for each group were similar (mean=57±3.5 mm³). Prior to each antibody administration, all mice received a 500 mg/kg (˜0.1 mL) intraperitoneal (IP) injection of pooled human antibodies (IVIG). mAbXcite-cetuximab, cetuximab (5 mg/kg) and control were administered via an IP injection a minimum of 2 hours post-IVIG administration. Throughout the study, tumors were monitored twice weekly to determine tumor growth rates using external calipers. Tumors were measured prior to and following the first day of treatment. Body weights were also collected twice a week. Mice were euthanized when the maximum tumor volume reaches 2000 mm³. Mice were also to be euthanized if the tumor became ulcerated, if the tumor impeded ambulation, or there was a deterioration of body condition. Mice were also to be euthanized if they lost >15% of their original body weight.

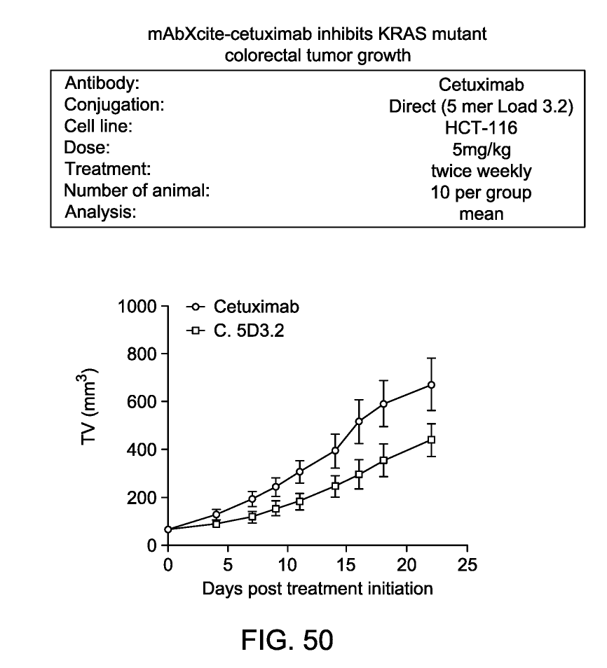

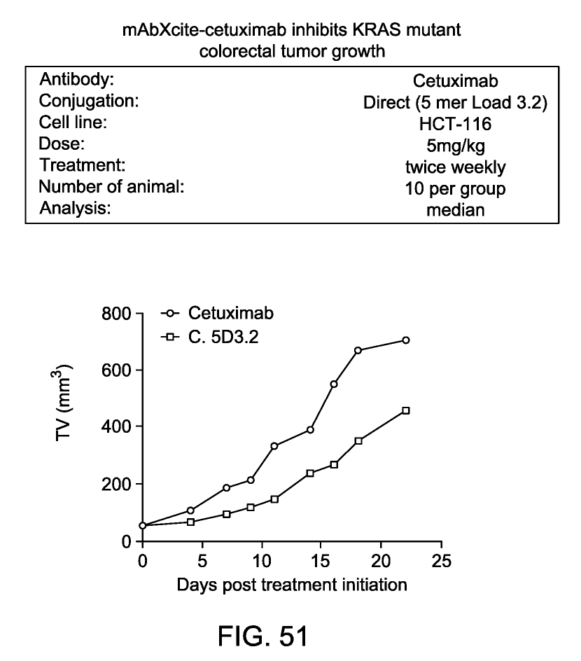

Analysis of data collected form this experiment was performed. Tumor volume was calculated based on the following formula: TV (mm³)={length (mm)×width (mm)²}/2. Results are shown in FIGS. 50 and 51. mAbXcite-cetuximab treatment with a 5-mer direct conjugate showed enhanced tumor growth delay on average and also by looking at the median.

### Example 16: Dose Response in Nude Mice with KRAS CRC

In this Example, the dose response of mAbXcite-cetuximab was studied. The present study utilized mAbXcite-cetuximab in which the conjugation utilized reductive amination (direct) chemistry, a 5-mer oligomer, and a load of about 3 oligomers per antibody.

In this Example, 7 week old female nude mice (Charles River) were implanted SC with 2.5×10⁶HCT-116 cells (HCT-116 is a human KRAS mutant colorectal cancer cell line). When tumor volume wan in the range of 51-80 mm³ mice were randomly divided into groups (9 mice/group) and treated with IVIG (as a source for anti-β-1,6-glucan antibodies) followed 2 hours later by vehicle control, cetuximab or mAbXcite EGFR. The dosing was determined by the half-life and was twice weekly. Various doses were assessed: 5, 10, 15 mg/kg for each antibody. Tumor volume was assessed by caliper measurement twice weekly. Body weight was monitored twice weekly. Mice were euthanized after tumors reached a volume of 2000 mm³ or if their body weight dropped by more than 15%.

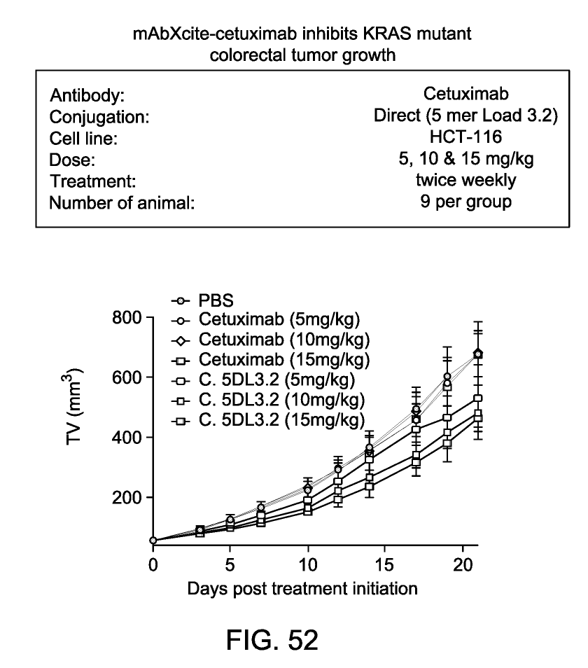

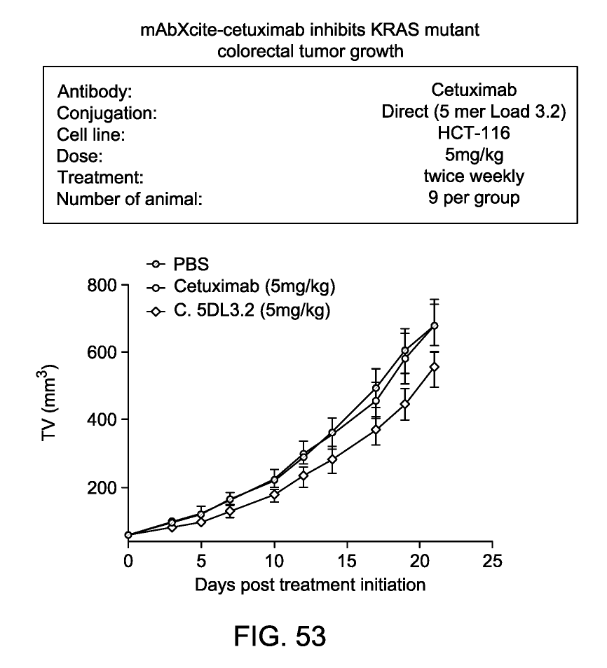

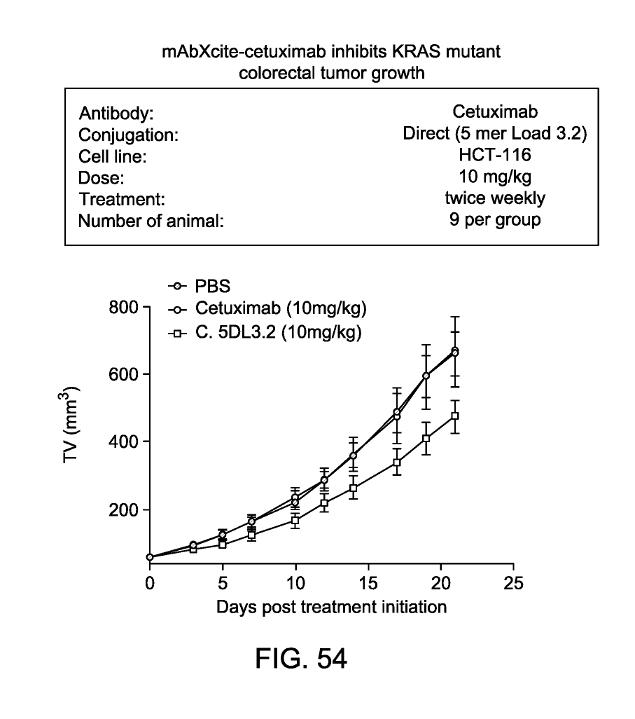

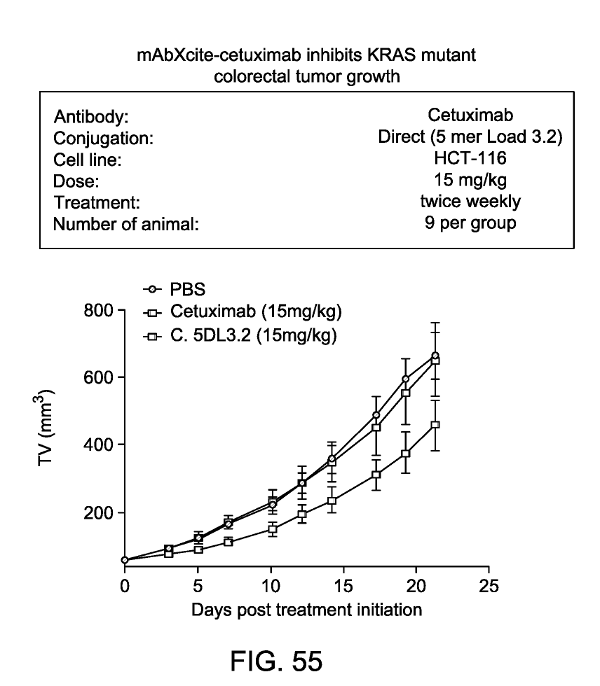

Analysis of data collected form this experiment was performed. Tumor volume was calculated based on the following formula: TV (mm³)={length (mm)×width (mm)²}/2. Results are shown in FIGS. 52 to 55.

### Example 17: Dose Response in Nude Mice with KRAS CRC

In this Example, mAbXcite-cetuximab treatment with a 5-mer direct conjugate demonstrated a dose-dependent tumor growth delay. The present study utilized mAbXcite-cetuximab in which the conjugation utilized reductive amination (direct) chemistry, a 5-mer oligomer, and a load of about 3 oligomers per antibody. This study utilized nude mice with KRAS CRC—repeat.

In this Example, 7 week old female nude mice (Charles River) implanted with 3×10⁶HCT-116 cells (HCT-116 is a human KRAS mutant colorectal cancer cell line). Once tumors reached approximately 65-89 mm³, mice were randomly assigned into four study treatment groups (n=7), such that the mean tumor volumes for each group were similar (mean=74±1.2 mm³). Prior to each antibody administration, all mice received a 500 mg/kg (˜0.1 mL) intraperitoneal (IP) injection of pooled human antibodies (IVIG). mAbXcite-cetuximab, cetuximab (5 mg/kg) and control were administered via an IP injection a minimum of 2 hours post-IVIG administration. Throughout the study, tumors were monitored twice weekly to determine tumor growth rates using external calipers. Tumors were measured prior to and following the first day of treatment. Body weights were also collected twice a week. Mice were euthanized when the maximum tumor volume reached 2000 mm³. Mice were also to be euthanized if the tumor became ulcerated, if the tumor impeded ambulation, or there was a deterioration of body condition. Mice were also to be euthanized if they lost >15% of their original body weight.

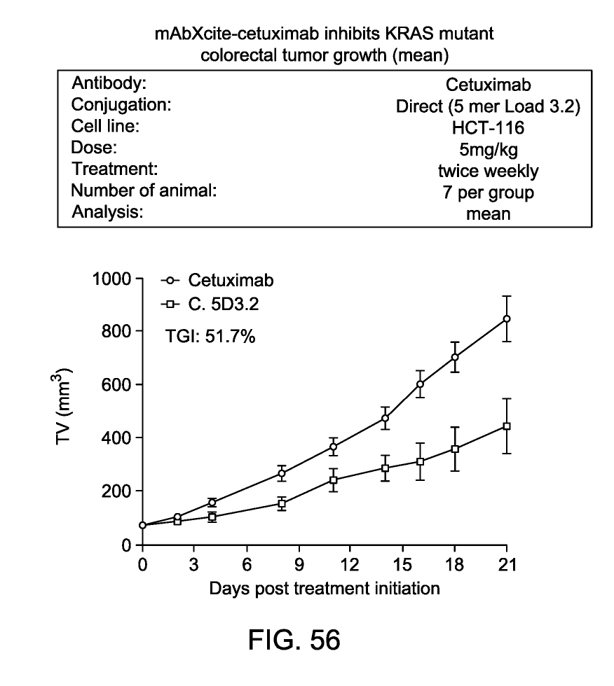

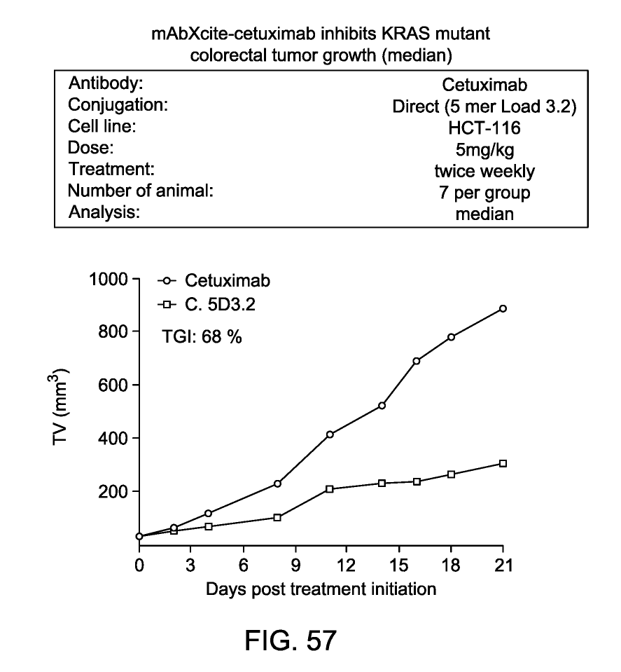

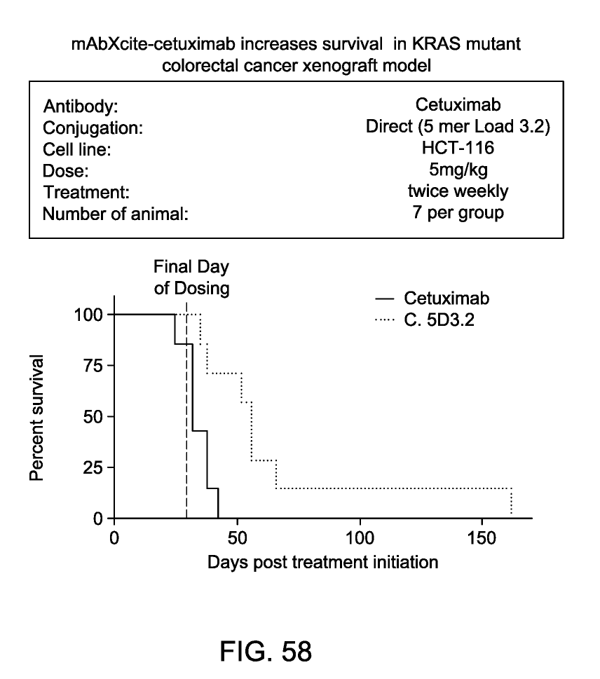

Analysis of data collected form this experiment was performed. Tumor volume was calculated based on the following formula: TV (mm³)={length (mm)×width (mm)²}/2. Tumor Growth Inhibition (TGI) was calculated based on the following formula: % TGI=(TVvehicle day x−TVvehicle day initial)−(TVtreatment day x−TVtreatment day initial)×100/(TVvehicle day x−TVvehicle day initial). A TGI greater than 50% is considered to be significant. Tumor volume results (see FIGS. 56 and 57) and a survival curve (see FIG. 58) are provided. mAbXcite-cetuximab treatment with a 5-mer direct conjugate showed enhanced tumor growth delay on average and also by looking at the median. This efficacy study in these HCT-116 tumor-bearing mice, receiving either cetuximab or mAbXcite-cetuximab showed longer survival in mice receiving mAbXcite-cetuximab compared to mice treated with cetuximab. This data suggests that mAbXcite-cetuximab specifically delivered to EGFR-positive tumors by cetuximab can suppress tumor growth despite the KRAS mutant status and present opportunities for personalized clinical treatment strategies in colorectal cancer.

### Example 18: Tumor Re-Challenge Assays in Nude Mice with KRAS CRC

Efficacy study in HCT-116 tumor-bearing mice, receiving mAbXcite-cetuximab (5-mer direct conjugation) showed longer survival in mice receiving mAbXcite-cetuximab than in mice treated with cetuximab. This survival was associated with mice exhibiting either regression or stasis of their tumors.

Since the generation of an adaptive immune memory response is essential for preventing tumor relapse and metastasis, we sought to address whether mAbXcite-cetuximab could also induce effective antitumor immune memory. To do so, we used tumor-rechallenge assays, as they are a robust readout not only for established memory responses but also for the ability of the host immune system to control systemic cancer relapse. Upon cessation of mAbXcite-cetuximab treatment, mice were re-challenged the with HCT-116 cells.

17 days after treatment cessation, nude mice were re-implanted subcutaneously (SC) with 2.5×10⁶ HTC-116 cells. Tumor volumes were assessed by caliper measurement twice weekly. Body weight monitored twice weekly. Mice were euthanized after tumors reached a volume of 2000 mm³ or if their body weight dropped by more than 15%.

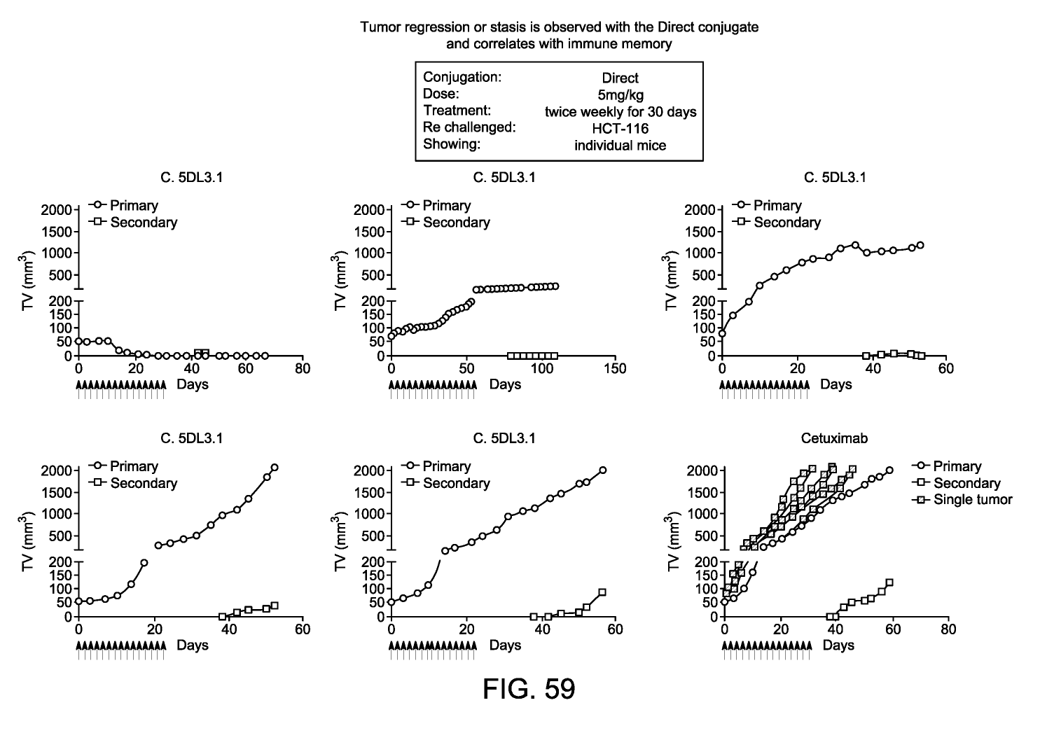

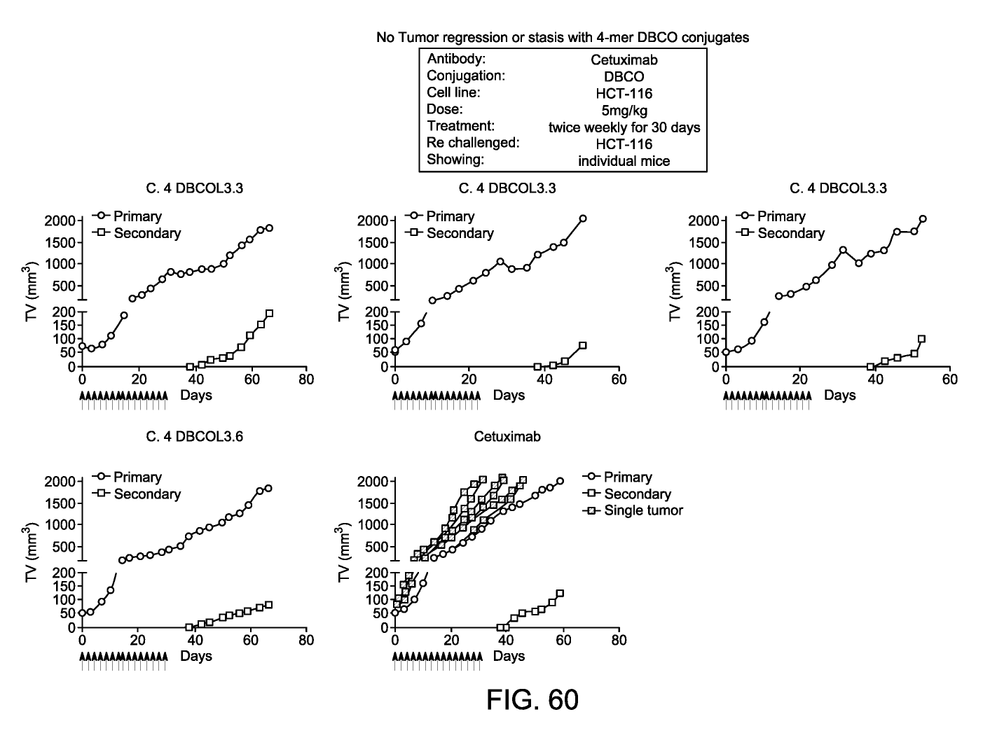

Tumor Volume was calculated based on the following formula: TV (mm³)={length (mm)×width (mm)²}/2. Results are shown in FIGS. 59 and 60 and show that mAbXcite-cetuximab inhibits tumor growth and protects mice from rechallenge with HCT-116. The primary tumor and secondary tumor are shown. Growth curves are shown for individual mice that were treated with 5 mg/kg of cetuximab (FIGS. 59 and 60), mAbXcite-cetuximab (5-mer direct conjugate in FIG. 59), or other constructs (FIG. 60).

Despite low levels of T cells present in nude mice, we showed that cetuximab directly conjugated to a 5-mer with a load of 3, leads to lasting effects in some of the mice. Specifically, we have demonstrated that mice that exhibit either regression or stasis of their tumors are not growing secondary tumors upon rechallenge with cancer cells, suggesting that neutrophils initiate a response that confers memory. This effect was not observed in mice initially treated with cetuximab or mAbXcite-cetuximab conjugated utilizing the click DBCO linker.

### Example 19: T-Cell Depletion in Nude Mice with BRAF CRC

Since mAbXcite-cetuximab shows efficacy and antitumor immune memory, we sought to address whether the overall antitumor effect of mAbXcite-cetuximab direct conjugate depends on cytotoxic T-lymphocyte (CTL). CD8⁺ T lymphocytes are the major cell population involved in controlling the growth of many tumors. However, to address whether CTL are essential for the therapeutic effect of mAbXcite-cetuximab, both CD8 and a CD4-depleting antibody were administered.

CD8⁺ CD4⁺-depleting antibody were administered to three mice, two of which had tumors that completely regressed and one whose tumor still showed stasis over 40 days upon treatment cessation and after being re-challenged. CD8 and a CD4-depleting antibody were administered twice weekly for the duration of the experiment. Tumor volume was assessed by caliper measurement twice weekly. Body weight was monitored twice weekly. Mice were euthanized after tumors reached a volume of 2000 mm³ or if their body weight dropped by more than 15%.

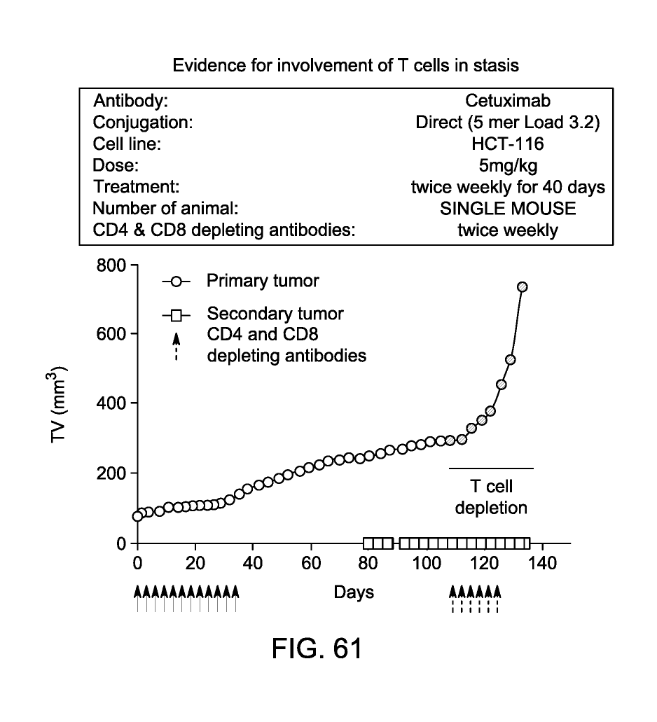

Tumor Volume was calculated based on the following formula: TV (mm³)={length (mm)×width (mm)²}/2. The two mice whose tumors regressed were not affected by the depletion. However, the one slow growing tumor grew exponentially following the depletion. Results are shown in FIG. 61. Results demonstrate that loss of CD8⁺ CD4⁺-cells significantly impaired the therapeutic effect of mAbXcite-cetuximab, suggesting that the antitumor effect of mAbXcite-cetuximab direct conjugate is indeed dependent on adaptive immune T cells, and that activation of neutrophils of the innate immune system progresses to activation of T cells of the adaptive immune system.

### Example 20: Efficacy Study in Nude Mice with KRAS CRC

In this Example, the efficacy of mAbXcite-cetuximab was studied in nude mice with KRAS CRC. The present study utilized mAbXcite-cetuximab in which the conjugation utilized reductive amination (direct) chemistry, a 4-mer or 6-mer oligomer, and a load of 2.3, 2.5, or 5 oligomers per antibody.

In this Example, 7 week old female nude mice (Charles River) implanted with 2.5×10⁶HCT-116 cells (HCT-116 is a human KRAS mutant colorectal cancer cell line). Once tumors reached approximately 46-79 mm³, mice were randomly assigned into four study treatment groups, such that the mean tumor volumes for each group were similar. Prior to each antibody administration, all mice received a 500 mg/kg (˜0.1 mL) intraperitoneal (IP) injection of pooled human antibodies (IVIG). mAbXcite-cetuximab, cetuximab (5 mg/kg) and control were administered via an IP injection a minimum of 2 hours post-IVIG administration. Throughout the study, tumors were monitored twice weekly to determine tumor growth rates using external calipers. Tumors were measured prior to and following the first day of treatment. Body weights were also collected twice a week. Mice were euthanized when the maximum tumor volume reaches 2000 mm³. Mice were also to be euthanized if the tumor became ulcerated, if the tumor impeded ambulation, or there was a deterioration of body condition. Mice were also to be euthanized if they lost >15% of their original body weight.

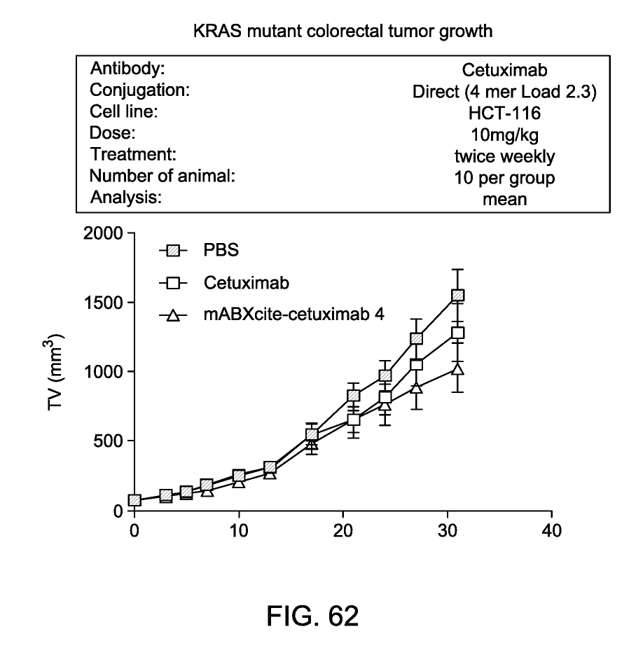

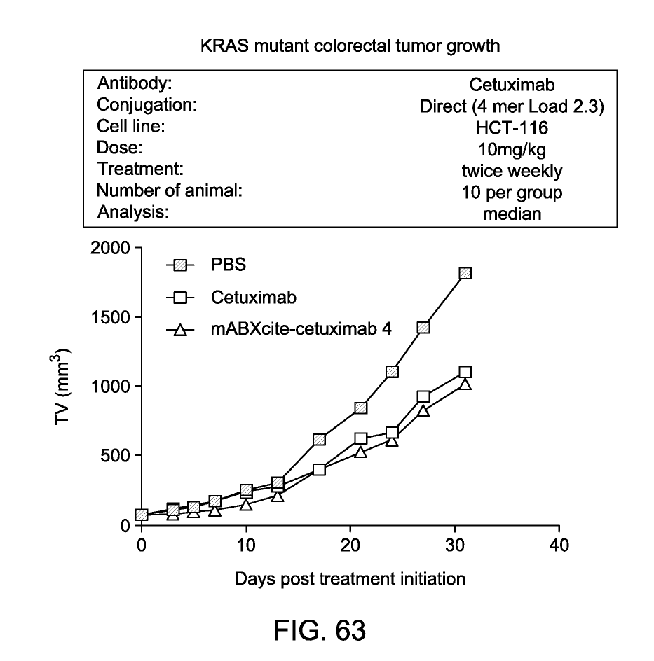

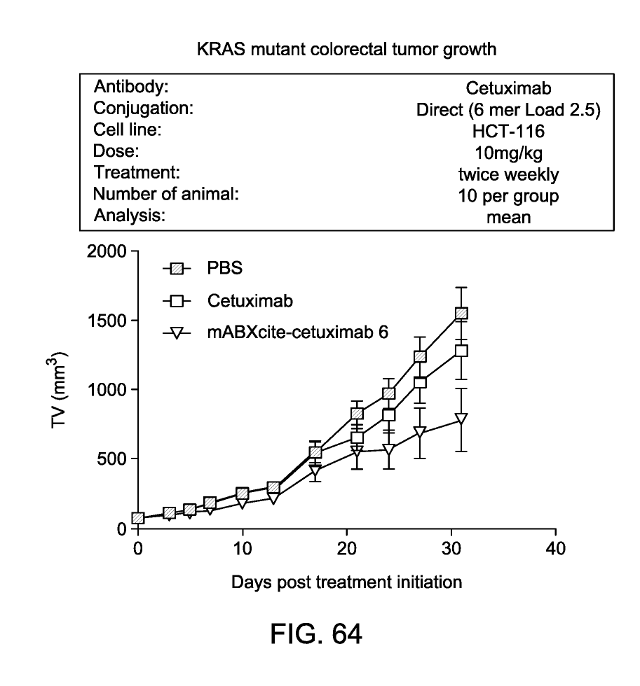

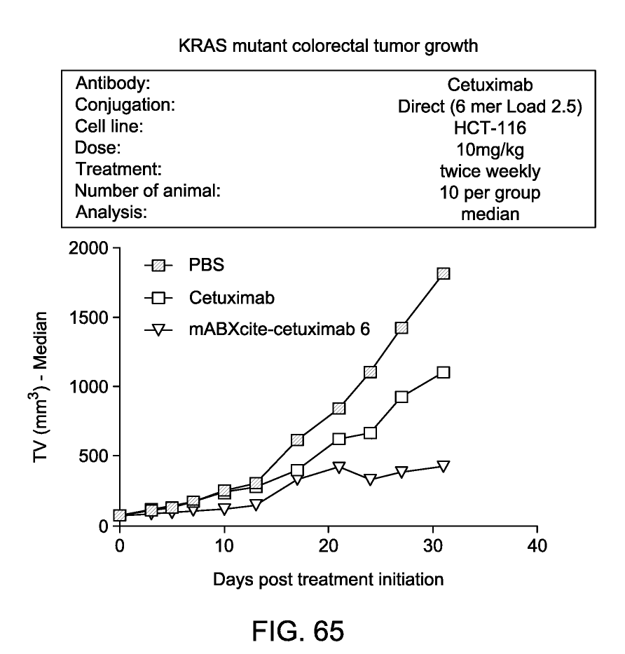

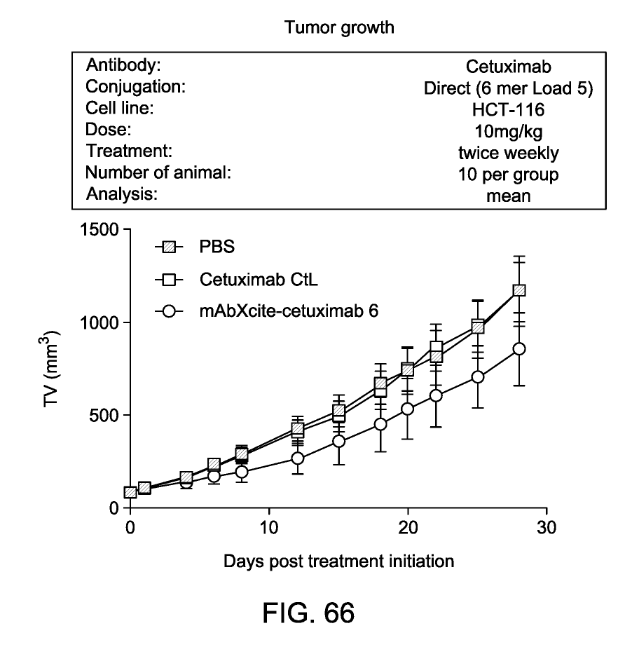

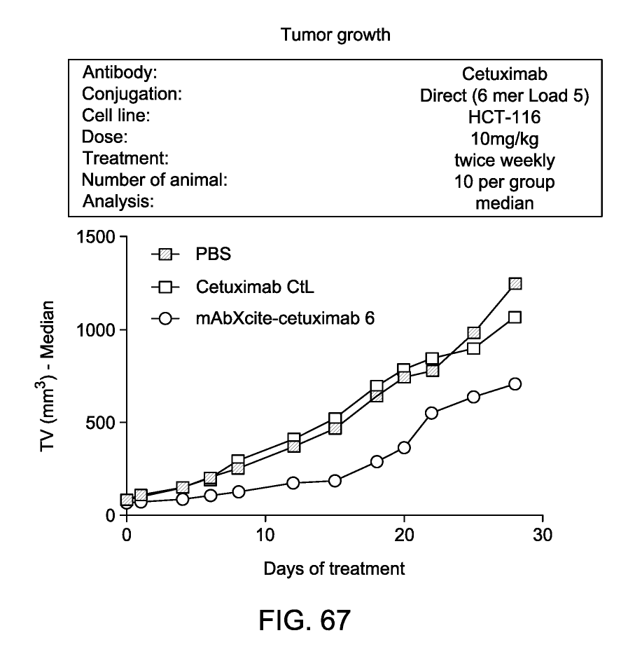

Analysis of data collected form this experiment was performed. Tumor volume was calculated based on the following formula: TV (mm³)={length (mm)×width (mm)²}/2. Results are shown in FIGS. 62 to 67.

### Example 21: Synthesis of Gentiopentose

The synthesis of the exemplary sugars below begin with the preparation of three “building blocks” (building block 1, building block 2, and building block 3). Two of the building blocks (building block 1 and building block 2) are protected dimers prepared from D-amygdalin. Building block 3 is prepared from glucose.

Scheme 1 illustrates the synthesis of building block 1.

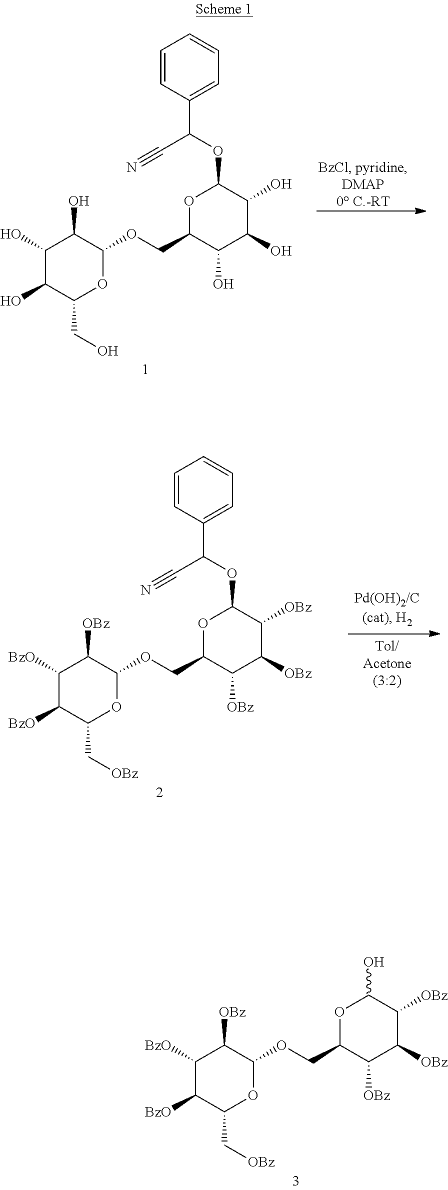

Compound 1 (D-amygdalin; commercially available from Carbosynth and Bosche Scientific) was reacted with benzoyl chloride in the presence of pyridine and DMAP to give benzoyl ester 2 (81% yield). Benzoyl ester 2 was dissolved in a 3:2 mixture of toluene and acetone, and reduced by exposure to a catalytic amount of Pd(OH)₂ on carbon, 50% wetted, in the presence of H₂ to give oligosacchiride 3 (93%) (“building block 1”). The overall yield for the two steps was 75%. Without being bound to any particular theory, it was believed that use of the benzoyl moiety as a protecting group of the hydroxyl groups on compound 1 facilitated selective incorporation of an ester adjacent to the site of glycosylation.

Scheme 2 illustrates the synthesis of building block 2.

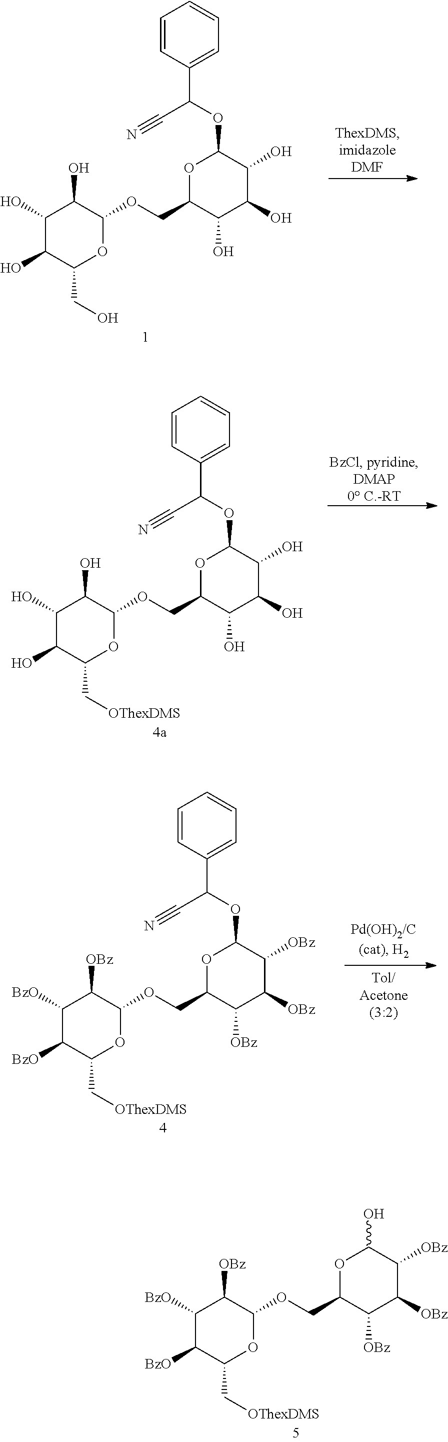

Compound 1 was first reacted with dimethylthexyl silyl chloride (ThexDMS-Cl) in the presence of imidazole and DMF to selectively protect the 6-hydroxyl moiety, giving compound 4a (82% yield). Compound 4a was then reacted with benzoyl chloride in the presence of pyridine and DMAP to give benzoyl ester 4 (81% yield). Without being bound to any particular theory, it is believed that use of ThexDMS was advantageous because the dimethyl-tert-butylsilyl moiety is easily cleaved during glycolysation. Benzoyl ester 4 was then dissolved in a toluene/acetone mixture (3:2 ratio), and reduced by exposure to a catalytic amount of Pd(OH)₂ on carbon, 50% wetted, in the presence of H₂ to give oligosacchiride 5 (93%) (“building block 2”). The overall yield for the process was 61%.

Scheme 3 illustrates the synthesis of building block 3.

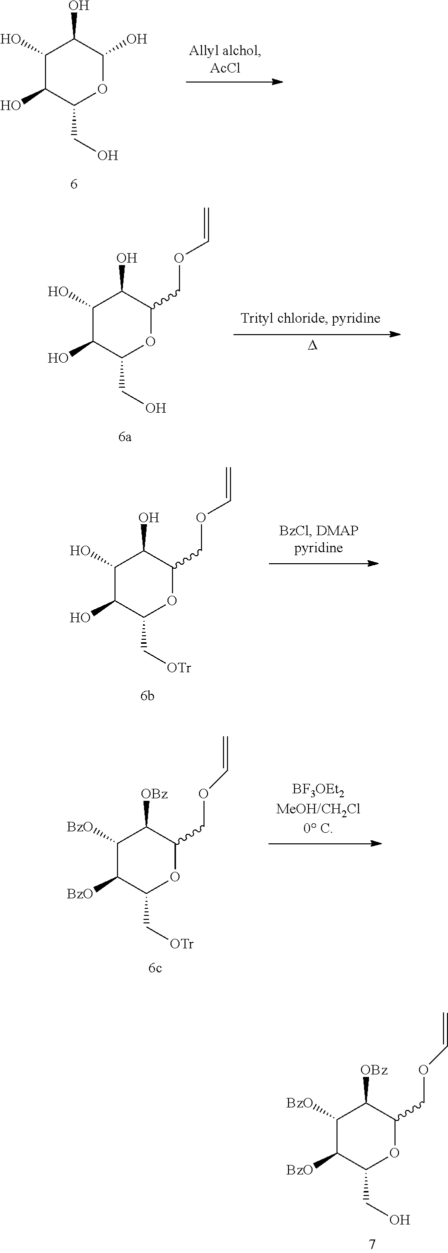

Compound 6 (glucose, commercially available from VWR) was first reacted with allylic alcohol in the presence of acetyl chloride to give compound 6a. Compound 6a was protected at the 6-hydroxyl position by reacting compound 6a with tritylchloride in the presence of pyridine and heat to give compound 6b. The remaining hydroxyl moieties of compound 6b were protected by reacting compound 6b with benzoyl chloride in the presence of pyridine and DMAP to give benzoyl ester 6c. Compound 6c was reacted with borontrifluoride etherate in a mixture of methanol and dichloromethane at 0° C. to give compound 7 (“building block 3”). The synthesis of building block 3 was performed with 42% overall yield.

Scheme 4 illustrates the synthesis of gentiotriose.

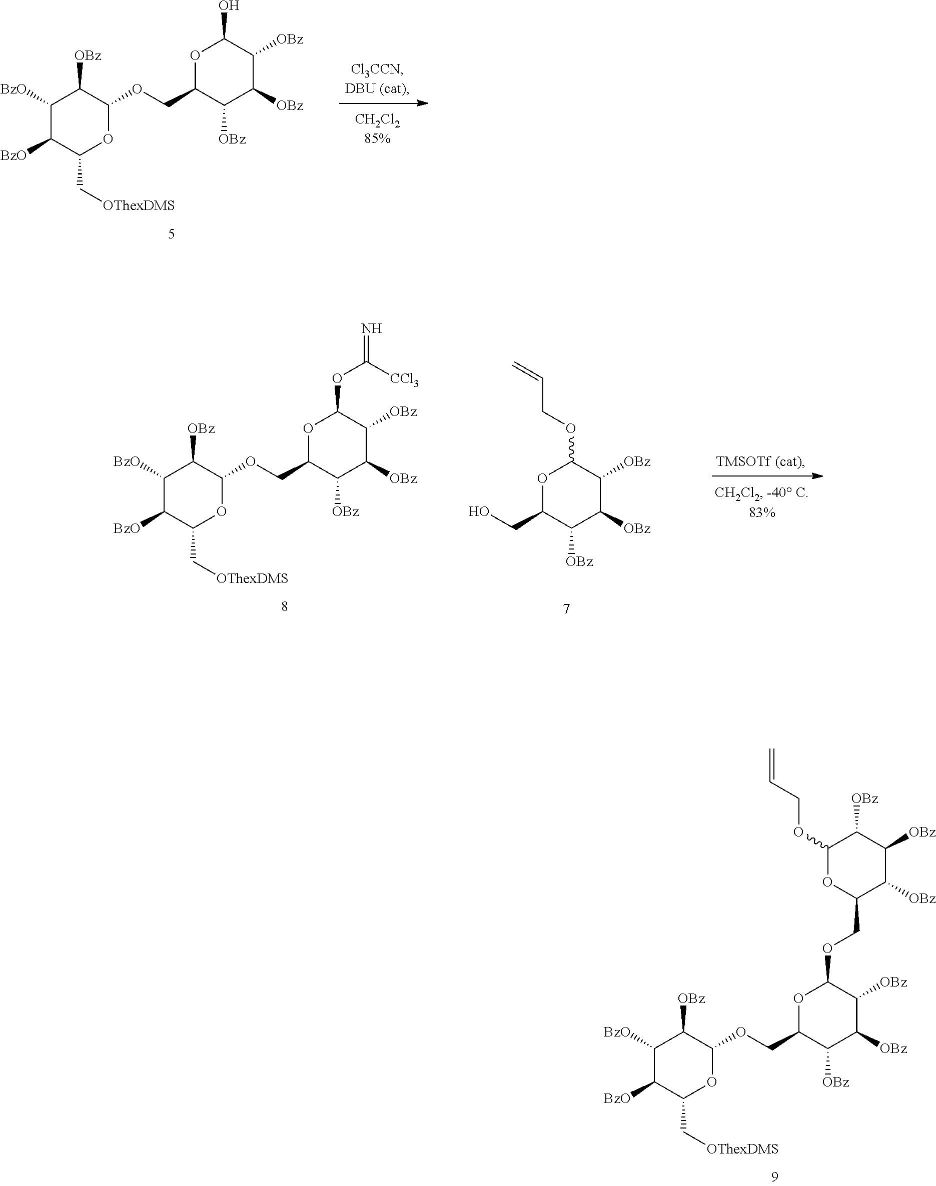

Compound 5 was reacted with Cl₃CCN in the presence of catalytic DBU in dichloromethane to give trichloroimidate 8. Compound 8 was repassed through a plug of base deactivated silica to remove base-line materials and carried forward without any further manipulation. Compound 8 was reacted with compound 7 (building block 3) under Schmidt glycosylation conditions (reacting the components with catalytic trimethylsilyl trifluoromethanesulfonate at −40° C.) to give compound 9 (gentiotriose). The overall yield for this two-step process was 70%.

Scheme 5 illustrates the glycosylation to form gentiopentose 12.

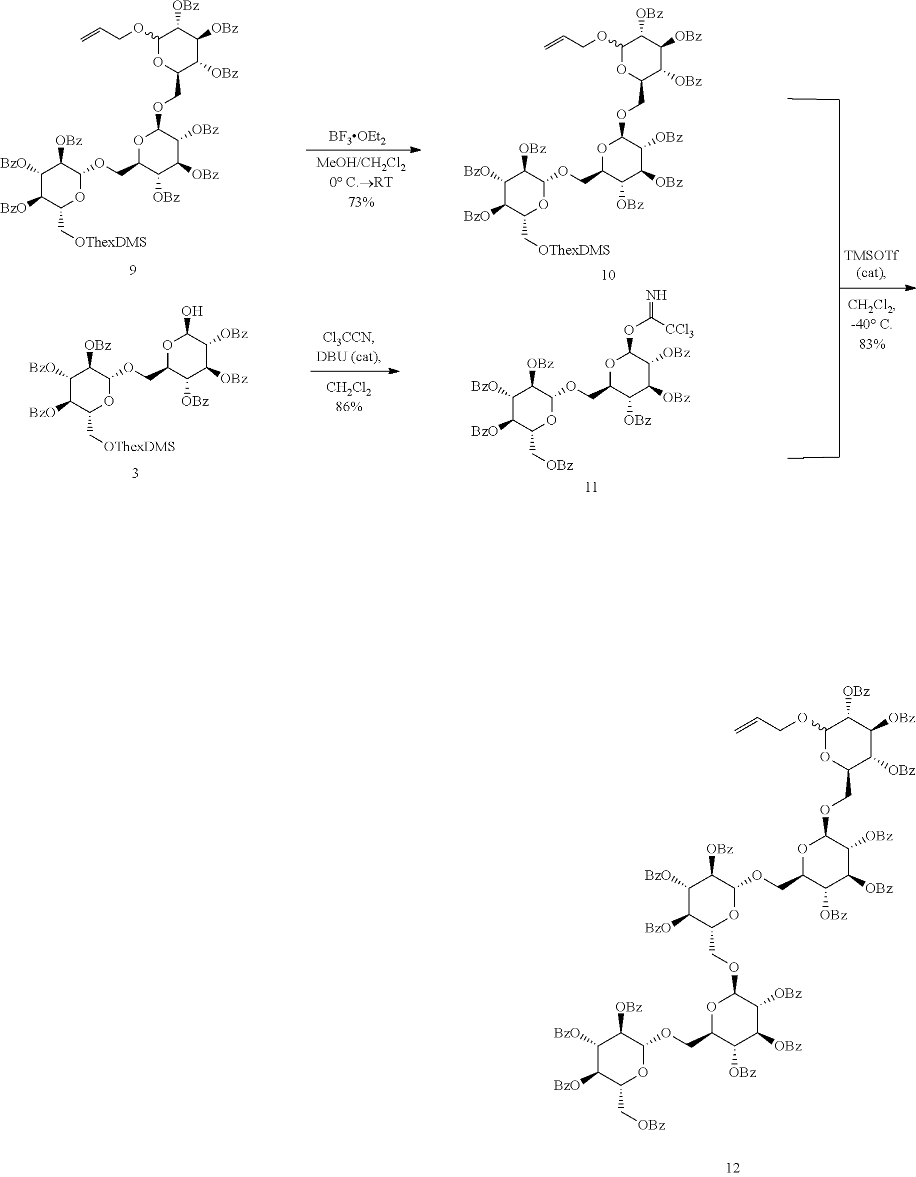

Gentiotriose 9 was treated with borontrifluoride etherate in methanol at 0° C. followed by warming to room temperature to afford the compound 10. Compound 3 (building block 1) was reacted with Cl₃CCN in the presence of catalytic DBU in dichloromethane to give trichloroimidate 11. Trichloroimidate 11 and compound 10 were reacted under Schmidt glycosylation conditions to afford the fully protected gentiopentose 12. The overall yield for these steps were 60%.

Scheme 6 illustrates the deprotection of gentiopentose 12.

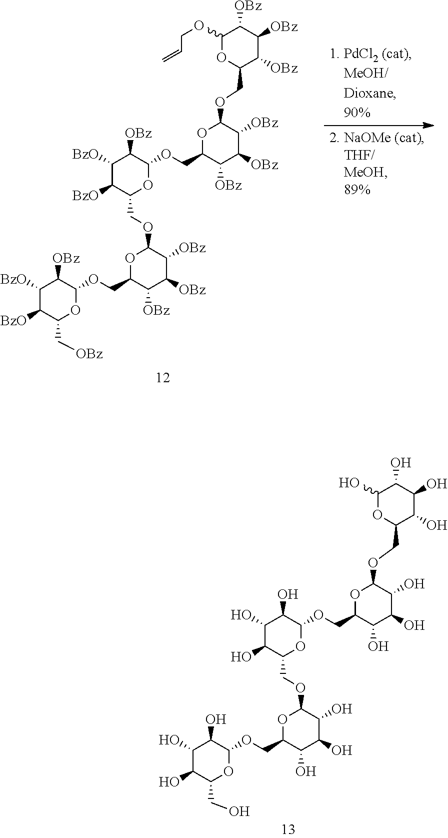

Gentiopentose 12 was contacted with a catalytic amount of palladium chloride in a mixture of methanol and dioxane to remove the allyl moiety. The resulting product was globally deprotected by reacting the product with a catalytic amount of sodium methoxide in a mixture of methanol and THF to provide gentiopentose 13. The overall yield for the 10 linear steps (16 total steps) was 14%.

### Example 22: Synthesis of 2-Azidoethylgentiotetrose

Scheme 7 illustrates the incorporation of the azido moiety into building block 2.

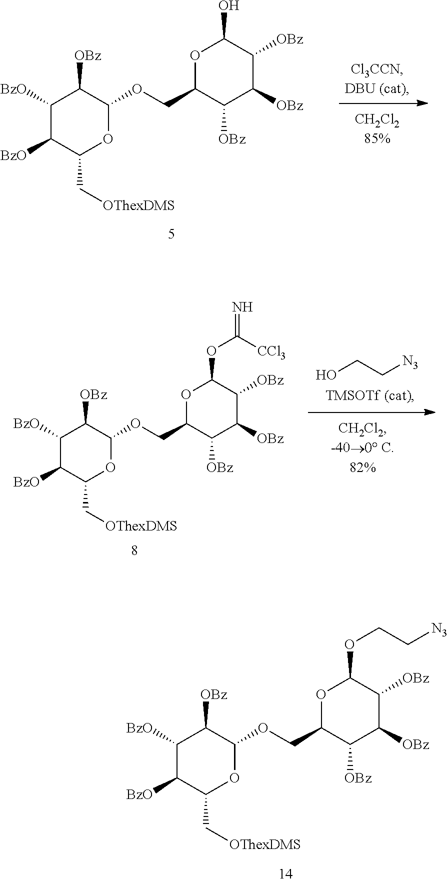

Compound 5 (building block 2) was reacted with Cl₃CCN in the presence of a catalytic amount of DBU in the presence of dichloromethane to give trichloroimidate 8. Trichloroimidate 8 was reacted under Schmidt glycosylation conditions to give compound 14. Compound 14 was synthesized in 70% overall yield.

Scheme 8 illustrates the synthesis of 2-azidoethylgentiotetrose.

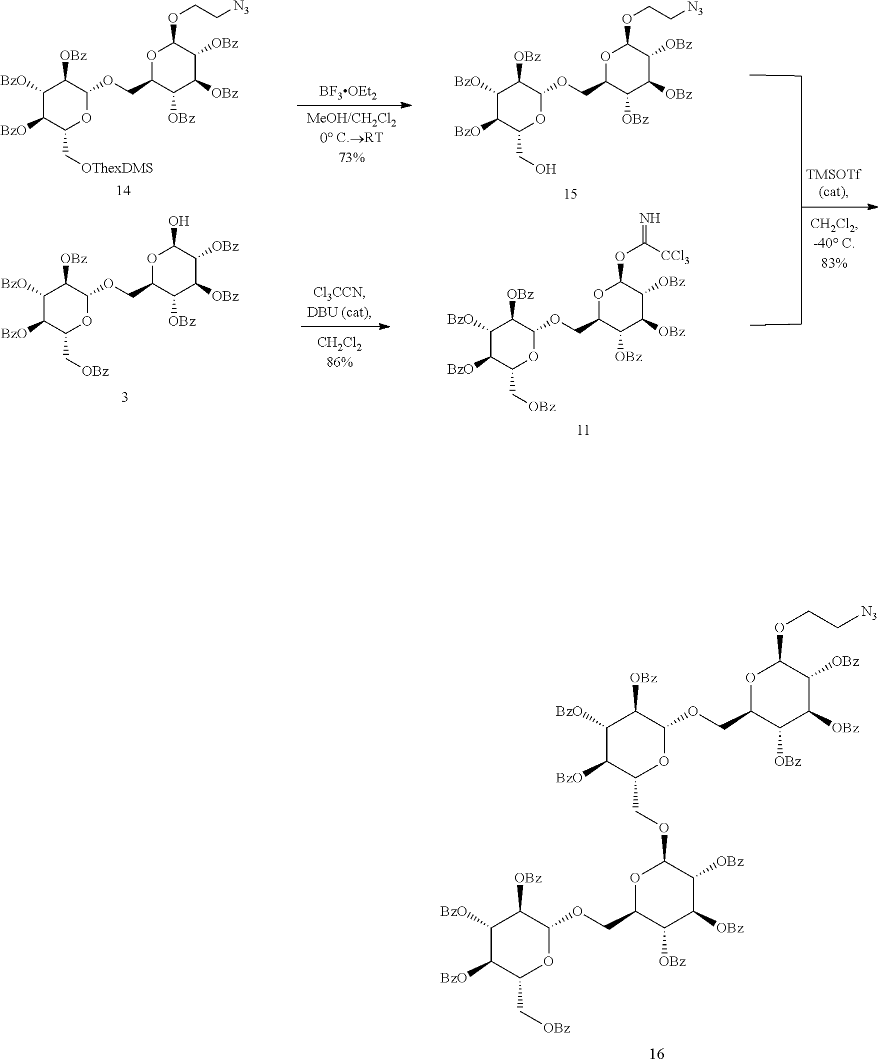

Compound 14 was deprotected by treatment of borontrifluoride etherate complex in methanol to give compound 15. Compound 3 (building block 1) was treated with trichloroacetonitrile and catalytic DBU to give compound 11. Compound 14 and compound 11 then subjected to the Schmidt glycosylation conditions to afford the gentiotetrose product 16. The overall yield of the sequence was 60%.

### Example 23: Synthesis of 2-Azidoethylgentiohexose

Scheme 9 illustrates the synthesis of gentiotriose 17.

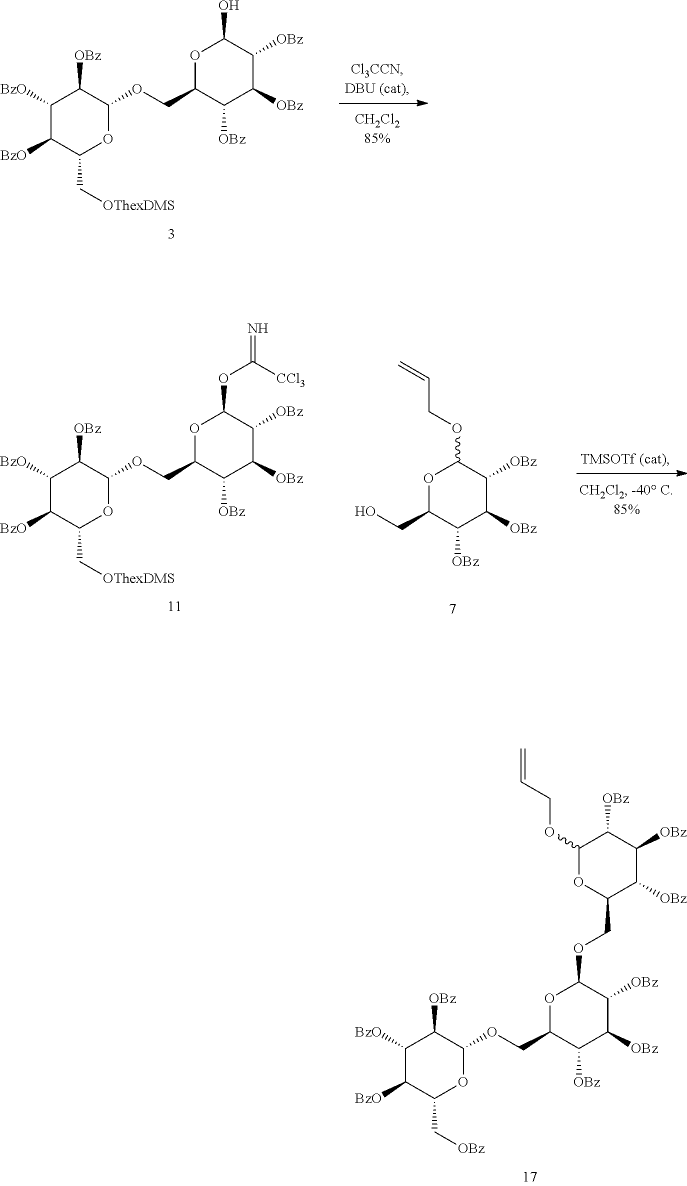

Compound 3 (building block 1) was reacted with Cl₃CCN in the presence of catalytic DBU in dichloromethane to give trichloroimidate 11. Trichloroimidate 11 was reacted with compound 7 (building block 3) under Schmidt glycosylation condition to give gentiotriose 17 (72% overall yield).

Scheme 10 illustrates incorporate of the 2-azidoethyl moiety into gentiotriose 9.

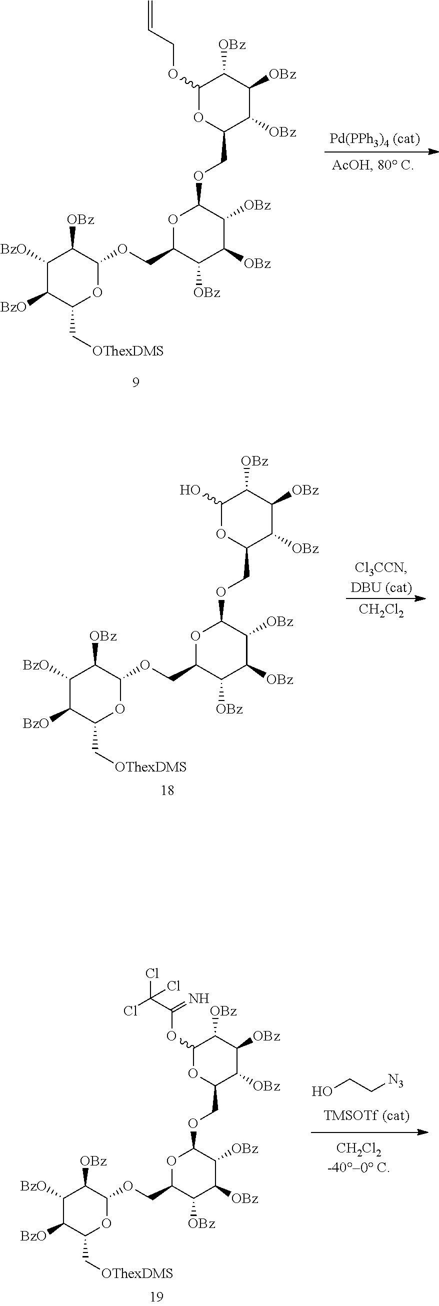

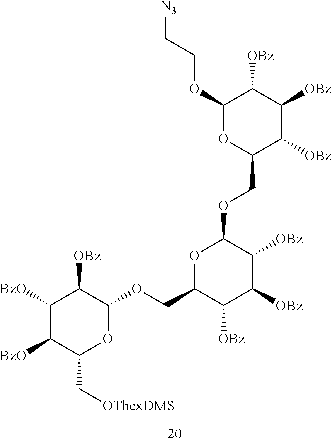

Gentiotriose 9 was deprotected by exposure to a catalytic amount of palladium (0) tetrakistriphenylphosphine in acetic acid at 80° C. to afford the compound 18. Utilization of palladium chloride in methanol resulted in the loss of the silyl moiety in addition to reduction of the allyl moiety. The glycosylation of the 2-azidoethanol under typical lewis acid conditions occurs after functionalization of the free hydroxyl moiety as the trichloroimidate 19 to afford the product 20. This sequence yielded 56% overall yield of 2-azidoethylgentiotriose.

Schemes 11a-c illustrates the synthesis of 2-azidoethylgentiohexose 24.

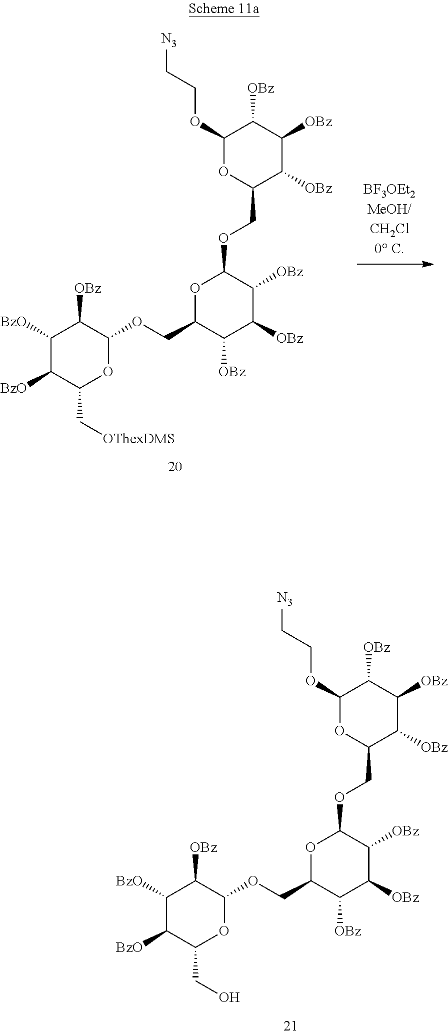

As illustrated in Scheme 11a, compound 20 was reacted with a borontrifluoride etherate complex in methanol to afford compound 21 (75% yield).

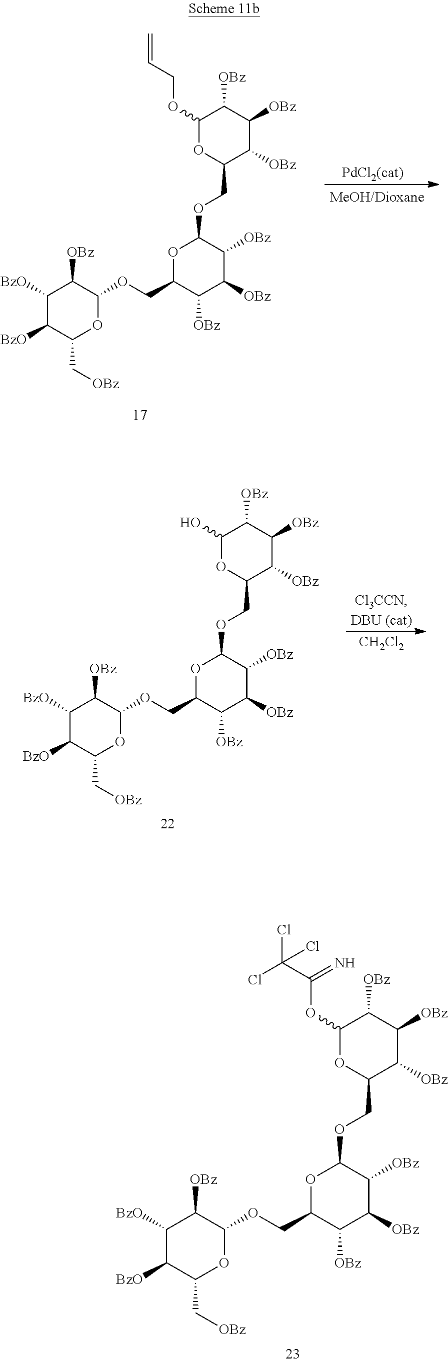

As illustrated in Scheme 11b, compound 17 was reduced with palladium (II) chloride in a mixture of methanol and dioxane to provide compound 22 (92% yield). Compound 22 was reacted with Cl₃CCN in the presence of catalytic DBU in dichloromethane to give trichloroimidate 23 (86% yield).

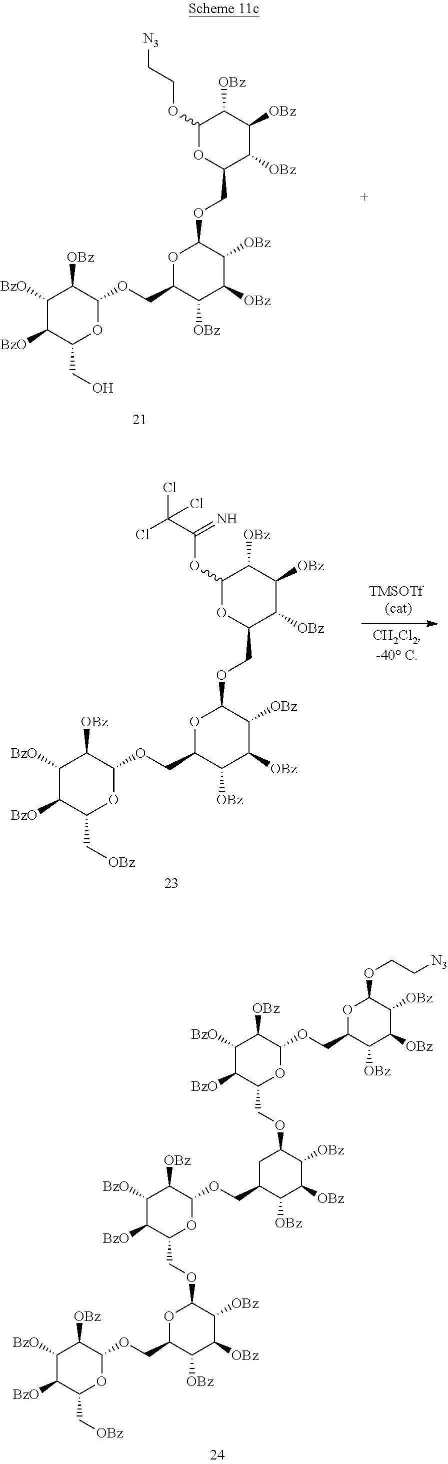

As illustrated in Scheme 11c, compounds 21 and 23 were under Schmidt glycosylation conditions to afford the desired product 24 (83% yield). The total synthetic process provided compounds 24 in 62% yield.

### Example 24: Generation of the DBCO NHS Esters

Scheme 12 illustrates the synthesis of NHS DBCO precursors 27 and 28.

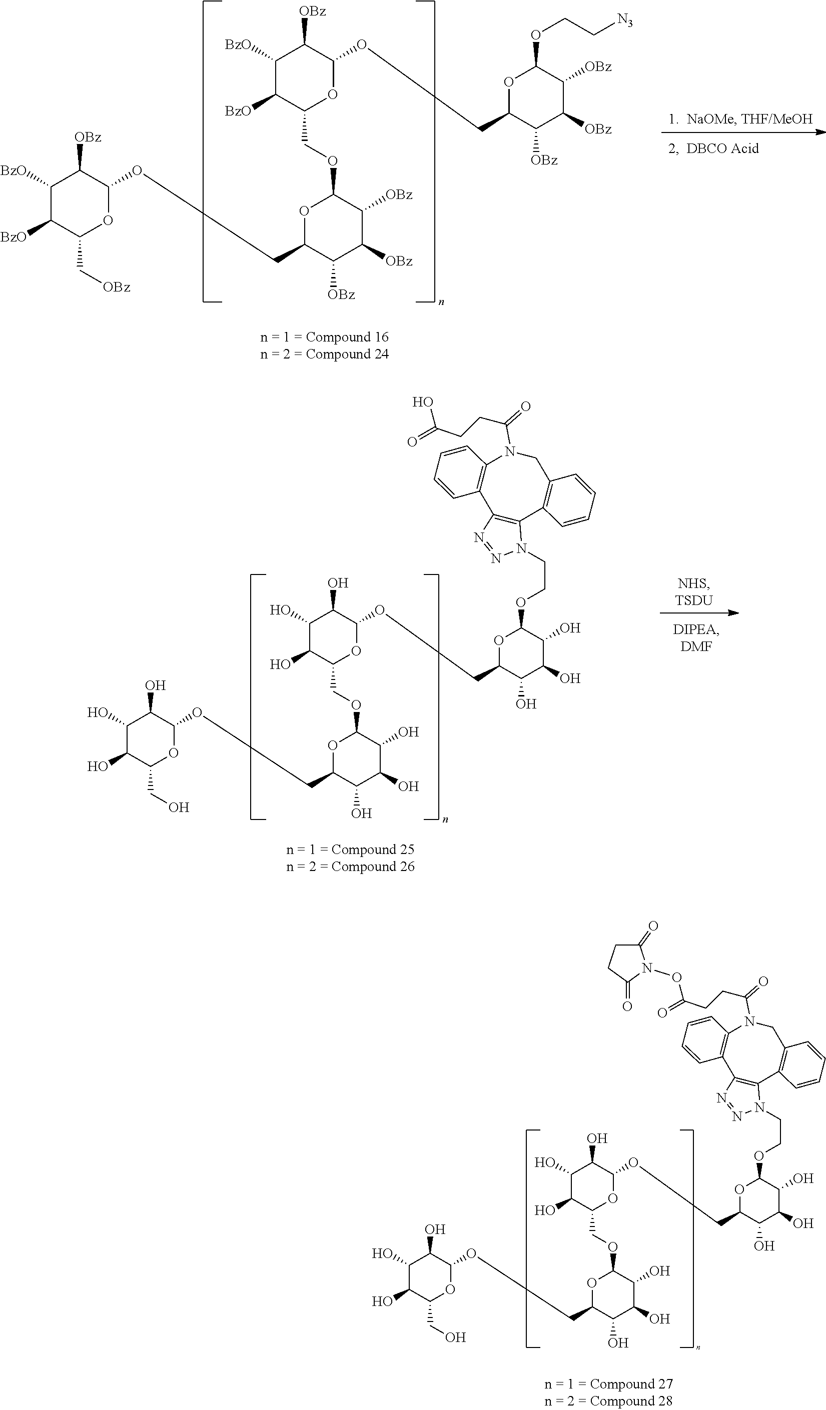

The protected oligosaccharides (either tetrose 16, when n=1, or hexose 24, when n=2) were deprotected by contacting the compound with catalytic NaOMe in a mixture of methanol and THF in 86% yield. The products were then subjected to click reaction conditions (reaction with dibenzocyclooctyllic acid (DBCO acid)) to provide compounds 25 (for n=1) and 26 (for n=2) (76% yield). No copper was needed to generate the mixture of triazole products (only one triazole isomer shown; however, a 1:1 ratio of isomers was noted by LC analysis) from the cylooctyne intermediate. Compounds 25 and 26 were reacted with NHS and TSDU in DMF under basic conditions to provide the NHS esters 27 (n=1) and 28 (n=2) (95% yield). The overall yield for this sequence was 62%.

Scheme 13 illustrates an alternative preparation of NHS-DBCO oligosaccharide.

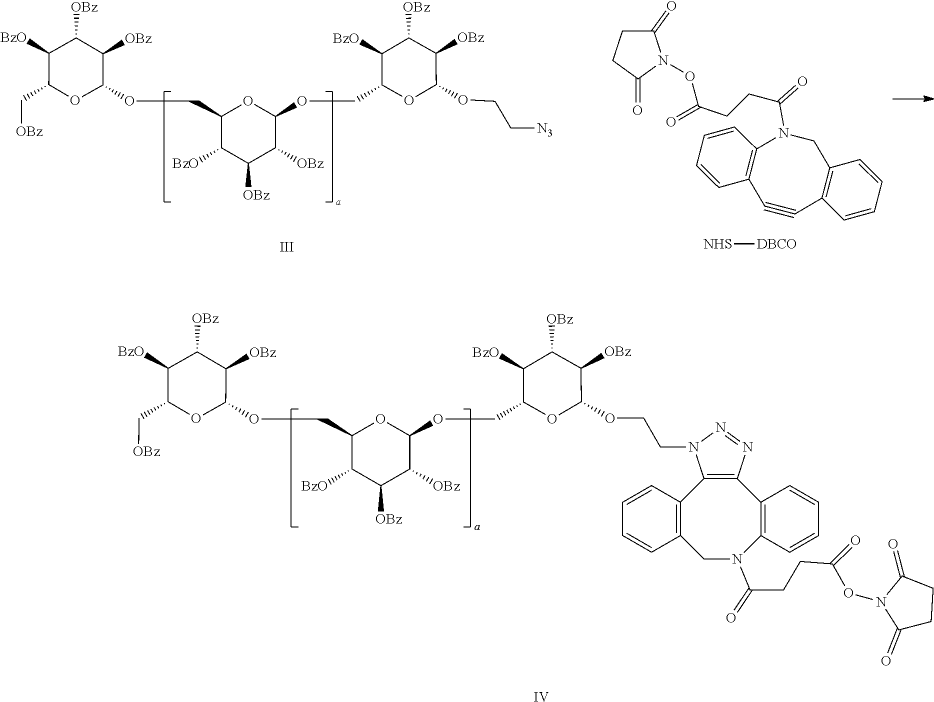

A 0.1 M solution of the compound of Formula III in endotoxin free was provided, wherein a is an between 0 and 8. 50 nM of NHS-DBCO in 100% DMSO was mixed into a solution of 40% DMSO/60% lx PBS (pH 6.0), followed by addition of the solution comprising formula X. The ratio of the compound of Formula III to NHS-DBCO was 1.5. The reaction was allowed to proceed at 21° C. and was monitored by LC-MS using a Polaris C18-A 4.6 mm×15 cm, with a 5 μm particle size (Agilent) to detect NHS-DBCO consumption. The reaction yielded compound of Formula IV.

### Example 25: Conjugation of β-1,6-Glucan Oligomers to Cetuximab

### Strategy 1: Conjugation of β-1,6-Glucan Oligomers to Cetuximab Via NHS-DBCO

The compound of Formula IV (Scheme 13) was conjugated to cetuximab (1 mg/mL in PBS, pH 7.2) by adding a 12× excess of the compound of formula X1 to a reaction solution of PBS (pH 7.2 containing 3% DMSO) and allowing the reaction to stir for 30 minutes at 21° C. The reaction was quenched by adding a 100-fold ratio of lysine to NHS-DBCO-oligosaccharide. Then, the mixture was buffer-exchanged using PBS, pH 7.2 with a 50 kDa TFF membrane. Final Cetuximab-oligosaccharide conjugate was adjusted to 1 mg/ml. Storage buffer is PBS, pH 7.2 at 4° C. Unconjugated oligosaccharide <0.1% of the concentration of oligosaccharide-Cetuximab conjugate.

### Strategy 2: Conjugation of β-1,6-Glucan Oligomers to Cetuximab Via Reductive Amination

Alternatively, β-1,6-glucan oligomers were conjugated to cetuximab via reductive amination. The β-1,6-glucan oligomers were of the formula V:

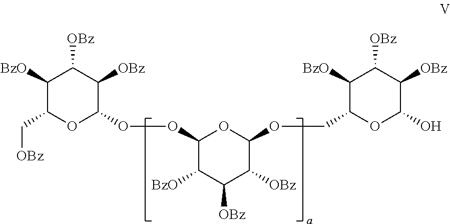

wherein a is between 0 and 8. Cetuximab was buffer exchanged from storage solution into 0.2 M Na-Borate, pH 8.0 by TFF (50 kDa membrane) and making the concentration of Cetuximab (10-20 mg/ml). The β-1,6-oligomers were prepared as a 0.1M solution in endotoxin-free water.
To a solution of 0.3 M sodium cyanoborohydride in endotoxin-free water, and 10:90 PEG 2000:endotoxin-free water was added 0.1M sodium borate until the pH was about 8.0. Cetuximab was added until the concentration of cetuximab was 5 mg/mL. A 230× molar excess of the β-1,6-oligomer solution was added at 21° C. and the reaction was allowed to stir for 23 hours. The conjugation is stopped by adding a 100-fold molar ratio of lysine to oligosaccharide. Reaction quench solution is 1 M lysine in 0.1 M Na-Borate, pH 8.0.

Following conjugation, the reaction mixture was diluted 1 to 10 in PBS, pH 7.2 and buffer-exchanged using PBS, pH7.2 with a 50 kDa TFF membrane. Final Cetuximab-oligosaccharide conjugate was adjusted to 1 mg/ml. Storage buffer was PBS, pH 7.2 at 4° C. Unconjugated oligosaccharide <0.1% of the concentration of oligosaccharide-Cetuximab conjugate. PEG of average MW 2000<0.1% of the concentration of oligosaccharide-Cetuximab conjugate.

Methods of Analysis:
a) SEC separation
   a. Unmodified Cetuximab
   b. Oligosaccharide-Cetuximab conjugate
   c. SEC separation utilizes a TSKgel SuperSW3000, 4 mm, 250 Å silica, 4.6 mm ID×30 cm using as a mobile phase 0.4M NaClO₄, 0.05M NaH₂PO₄, pH 7.2 utilizing UV detection @λ=280 nm
   b) Load estimation by MALDI (AB Sciex MALDI/Q-TOF 4800 or comparable instrument). Note: HIC is not able to resolve individual loads due to hydrophilicity of oligosaccharides
      a. Utilize BSA (MW 66341) as the calibration standard (AB Sciex) in linear high mass positive mode
      b. Utilize sinapinic acid (Sigma) as matrix
      c. Use native Cetuximab as control
      d. Estimate load by subtracting average m/z of Cetuximab conjugate from that of unmodified Cetuximab and divide by mass of a unit load to determine load of conjugate. Data has an average standard deviation of 0.2-0.3 load units
   c) Measure m/z of oligosaccharides by MALDI (AB Sciex 4800 or comparable instrument) using HABA (Sigma) as matrix.
   d) Measure m/z of oligosaccharides by LC-MS using)(Bridge 3 mm×10 cm, 3.5 μm particle size (Waters) using as mobile phase A: Water/0.1% Formic acid, B: 100% Acetonitrile/0.1% Formic acid. We use a ZQ (Waters) for mass spectrometry

### Example 26: Purification of Oligomers from Pustulan

### Degradation of Pustulan

Pustulan (Elicityl-Oligotech) was suspended in concentrated hydrochloric acid at 100 mg/ml. The resulting slurry was stirred vigorously at room temperature for 1.25 h, during which time the mixture's viscosity was noticeably reduced. The fine black slurry was then transferred to a beaker containing seven volumes of n-propanol, which led to the precipitation of a tan solid. After vigorous stirring for 15 min, the mixture was transferred to several tubes to separate the insoluble solids from the liquid by centrifugation (3600×g, 5 min). The supernatant was decanted leaving behind a light brown pellet. The pellets were successively washed with ethanol and n-propanol to remove traces of the lower order sugars (mono- and di-saccharides). Water was added to each tube and the subsequent mixture was stirred overnight at room temperature. Centrifugation (3600×g, 15 min) of these stirred mixtures yielded dark brown pellets and clear, lightly colored supernatant. The supernatants were pooled, frozen and lypholized yielding a ladder of β-1,6-glucans as a tan powder with a 15% by mass recovery based on starting pustulan. The resulting size of oligosaccharides ranged from 3mer to >14mer with the higher oligosaccharides (>14) present by LC but outside the detection limit of the MS, as determined by LC-MS analysis of the mixture via XBridge BEH HILIC Amide OBD Prep Column (XBridge BEH Amide HILIC OBD Prep Column, 130 Å, 5 μm, 19 mm×250 mm and XBridge BEH Amide HILIC analytical column, 3.5 μm, 3 mm×100 mm acquired from Waters, pre-washed as described by the manufacturer prior to use; 95-40% acetonitrile/water w/0.1% formic acid, 50 C, 0.75 mL/min for 15 min). In addition, the bulk of material consisted of 3mer to 8mer, as determined by ELSD analysis.

### Bulk Size Fractionation of the Oligosaccharide Ladder by P2

XK50 columns (P2 extra fine resin acquired from Biorad, prepared as described by the manufacturer, utilized to pack two XK50 100 cm long columns) were connected in parallel to an Agilent 1100 isocratic pump. A external pressure gauge was installed downstream of the pump to monitor the column pressure. All separations were carried out utilizing 0.1 M acetic acid with a flow rate of 3.5 ml/min. The oligosaccharide ladder was dissolved in water to a total volume of up to 12 mL and was injected onto the column via a manually injector possessing a 13 ml loop. The separation occurred over an approximately 500 minute period with the first 240 minutes diverted to waste. The remaining flow was collected as 6 mL fractions totaling 288 fractions. Fractions were analyzed by MALDI/TOF and fractions containing β-1,6-glucan of distinct sizes were pooled together, frozen and lypholized.

### Final Purification of Oligosaccharides Via HILIC and C18/C18AQ Chromatography

The dried samples from the P2 purification were dissolved in minimal amount of water and further separated on a)(Bridge BEH Amide HILIC OBD Prep Column (XBridge BEH Amide HILIC OBD Prep Column, 130 Å, 5 μm, 19 mm×250 mm and XBridge BEH Amide HILIC analytical column, 3.5 μm, 3 mm×100 mm acquired from Waters, pre-washed as described by the manufacturer prior to use; 130 Å, 5 μm, 19 mm×250 mm, 95-40% acetonitrile/water w/0.1% formic acid, 25 mL/min for 30 min). The purified fractions containing oligosaccharides of distinct sizes as determined by LC/MS were frozen and lypholized. After drying to completion, the oligosaccharides were redissolved in minimal water containing 0.1% formic acid and passed through a C18 AQ and a C18 column (C18 and C18AQ cartridges acquired from Teledyne ISCO and pre-washed as recommended by the manufacturer prior to use; 10× wt/wt bed size) linked in sequence and the product was eluted with water with 0.1% formic acid (10 column volumes). The flow through was frozen and lypholized to a white powder.

### Other Embodiments

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

What is claimed is:

1. A composition comprising a cetuximab antibody conjugated to between 1 and 6 β-1,6-glucan oligomers, wherein each β-1,6-glucan oligomer is independently comprised of between 2 and 10 glucose monomer units.

2. The composition of any preceding claim, wherein the β-1,6-glucan oligomers are independently comprised of between 3 and 7 glucose monomer units.

3. The composition of any preceding claim, wherein the β-1,6-glucan oligomers are independently comprised of between 3 and 5 glucose monomer units.

4. The composition of any preceding claim, wherein the β-1,6-glucan oligomers are each comprised of 4 glucose monomer units.

5. The composition of any preceding claim, wherein the antibody is conjugated to between 2 and 4 β-1,6-glucan oligomers.

6. The composition of any preceding claim, wherein the antibody is conjugated to 3 β-1,6-glucan oligomers.

7. The composition of any preceding claim, wherein the antibody is conjugated to between 2 and 4 β-1,6-glucan oligomers, and the β-1,6-glucan oligomers are independently comprised of between 3 and 7 glucose monomer units.

8. The composition of any preceding claim, wherein the antibody is conjugated to between 2 and 4 β-1,6-glucan oligomers, and the β-1,6-glucan oligomers are independently comprised of between 3 and 5 glucose monomer units.

9. The composition of any preceding claim, wherein the antibody is conjugated to between 2 and 4 β-1,6-glucan oligomers, and the β-1,6-glucan oligomers are each comprised of 4 glucose monomer units.

10. The composition of any preceding claim, wherein the antibody is conjugated to 3 β-1,6-glucan oligomers, and the β-1,6-glucan oligomers are independently comprised of between 3 and 7 glucose monomer units.

11. The composition of any preceding claim, wherein the antibody is conjugated to 3 β-1,6-glucan oligomers, and the β-1,6-glucan oligomers are independently comprised of between 3 and 5 glucose monomer units.

12. The composition of any preceding claim, wherein the antibody is conjugated to 3 β-1,6-glucan oligomers, and the β-1,6-glucan oligomers are each comprised of 4 glucose monomer units.

13. The composition of any preceding claim, wherein the antibody is conjugated to the β-1,6-glucan oligomers according to Formula II: 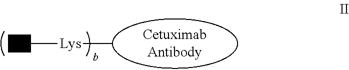
wherein:
Lys is a lysine residue;
b is between 1 and 6; and [symbol]
is a compound of Formula I: 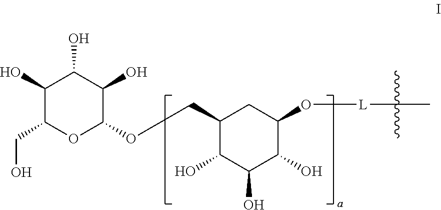
wherein:
a is between 1 and 9;
L is a linker; and
“ [symbol] ” represents a point of attachment between two atoms.

14. The composition of claim 13, wherein [symbol] is a compound of Formula Ia: 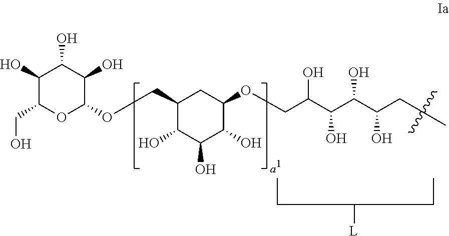
wherein:
a¹ is between 1 and 9; and
“ [symbol] ” represents a point of attachment between two atoms.

15. The composition any one of the preceding claims, wherein the antibody comprises a variable domain having at least 80% identity with SEQ ID NO: 4 or SEQ ID NO: 5.

16. The composition any one of the preceding claims, wherein the antibody comprises a heavy chain variable domain having at least 80% identity with SEQ ID NO: 4.

17. The composition any one of the preceding claims, wherein the antibody comprises a heavy chain having at least 80% identity with SEQ ID NO: 1.

18. The composition any one of the preceding claims, wherein the antibody comprises a light chain variable domain having at least 80% identity with SEQ ID NO: 5.

19. The composition any one of the preceding claims, wherein the antibody comprises a light chain having at least 80% identity with SEQ ID NO: 2.

20. The composition of any one of the preceding claims, wherein the antibody is cetuximab.

21. The composition of any one of the preceding claims, wherein the antibody competes with cetuximab for binding to EGFR.

22. The composition of any one of the preceding claims, wherein the β-1,6-glucan oligomers are chemically synthesized.

23. The composition of any one of the preceding claims, wherein at least 90% of the dry weight of glucan contained in the composition is β-1,6-glucan.

24. The composition of any one of the preceding claims, wherein less than 10% of the dry weight of glucan contained in the composition is β-1,3-glucan.

25. The composition of any one of the preceding claims, wherein the composition is substantially free of β-1,3-glucan.

26. A method of treating a cancer associated with expression of EGFR in a subject in need thereof, the method comprising administering to the subject a therapeutically effective amount of a composition of any one of the preceding claims.

27. The method of claim 26, wherein the cancer is a colorectal cancer.

28. The method of claim 26, wherein the cancer is a KRAS wild-type EGFR-expressing colorectal cancer.

29. The method of claim 26, wherein the cancer is a KRAS mutant EGFR-expressing colorectal cancer.

30. The method of claim 26, wherein the cancer is a BRAF mutant EGFR-expressing colorectal cancer.

31. The method of claim 26, wherein the cancer is a squamous cell carcinoma.

32. The method of claim 26, wherein the cancer is a squamous cell carcinoma of the head and neck.

33. The method of claim 26, wherein the cancer is a lung cancer.

34. The method of claim 26, wherein the cancer is a triple negative breast cancer.
